(19) United States
(12) Patent Application Publication
(10) Pub. No.: US 2026/0183275 A1
(43) Pub. Date: Jul. 2, 2026

(54) SUBSTITUTED QUINOLONE COMPOUNDS USEFUL AS INHIBITORS OF TLR9

(71) Applicant: Bristol-Myers Squibb Company, Princeton, NJ (US)

(72) Inventors: Michael MANDLER, Lawrenceville, NJ (US); Shoshana L. POSY, Highland Park, NJ (US); Yeheng ZHU, Stockton, NJ (US); Alicia REGUEIRO-REN, New Hope, PA (US)

(73) Assignee: Bristol-Myers Squibb Company, Princeton, NJ (US)

(21) Appl. No.: 19/128,242

(22) PCT Filed: Nov. 9, 2023
(86) PCT No.: PCT/US2023/079190

§ 371 Date: May 8, 2025

Related U.S. Application Data
(60) Provisional application No. 63/424,343, filed on Nov. 10, 2022.

(51) Int. Cl. A61K 31/4704; A61K 31/4709; A61P 1/16; C07D 215/227; C07D 215/38; C07D 401/04; C07D 401/10; C07D 401/14; C07D 405/12

(52) U.S. Cl. CPC A61K 31/4704; A61K 31/4709; A61P 1/16; C07D 215/227; C07D 215/38; C07D 401/04; C07D 401/10; C07D 401/14; C07D 405/12

(57) ABSTRACT

Disclosed are compounds of Formulas (I): (I) or a salt thereof, wherein L, R₁, R₂, R₃, and R₄ are defined herein. Also disclosed are methods of using such compounds as inhibitors of TLR9, and pharmaceutical compositions comprising such compounds. These compounds are useful in treating, preventing, or slowing fibrotic diseases.

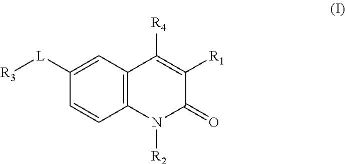

### CROSS REFERENCE

This application claims the benefit of U.S. Provisional Application Ser. No. 63/424,343 Nov. 10, 2022 which is incorporated herein in its entirety.

### DESCRIPTION

The present invention generally relates to substituted quinolone compounds useful as inhibitors of signaling through Toll-like receptor 9 (TLR9). Provided herein are substituted quinolone compounds, compositions comprising such compounds, and methods of their use. The invention further pertains to pharmaceutical compositions containing at least one compound according to the invention that are useful for the treatment of conditions related to TLR9 modulation, such as fibrotic diseases, and methods of inhibiting the activity of TLR9 in a mammal.

Toll-like receptors (TLRs) are transmembrane proteins having the ability to initiate an inflammatory response upon recognition of pattern-associated molecular patterns (PAMPs) or microbe-associated molecular patterns (MAMPs). A total of 10 human TLRs have been identified and can be located in the cell surface or, as in the case of TLR7, 8 and 9, in the endolysosomes. TLR9 recognizes unmethylated single-stranded DNA containing cytosine-phosphate-guanine (CpG) motifs that are typically found in bacterial and mitochondrial DNA (mtDNA). TLR9 may contribute to fibrogenesis by promoting inflammation via the MYD88-dependent signalling pathway that ultimately mediates activation of IL-6, IFN-α, IL-1β, and TNF-α among others cytokines. (Barton GM, Kagan JC (2009) Nat. Rev. Immunol. 9(8), 535-42; Li X, Jiang S, Tapping RI (2010) Cytokine 49(1), 1-9).

TLR9 levels are higher in lung biopsies of rapid idiopathic pulmonary fibrosis (IPF) progressors than in the healthy or stable IPF progressors (Sci. Transl. Med. 2010, 2(57):57ra82). Circulating mtDNA, the ligand for TLR9 has recently been identified as a mechanism-based prognostic biomarker of IPF (Am J. Resp. and Crit. Care Med. 2017, 196(12), 1502). In addition, it has been observed that TLR9 is up-regulated in human and murine non-alcoholic steatohepatitis (NASH) (Clin. Sci. 2017, 131(16), 2145), while hepatocyte mitochondrial DNA drives NASH via activation of TLR9 (J. Clin. Inv. 2016, 126(3), 859. Accordingly, inhibitors/antagonists of TLR9 are predicted to have efficacy as novel therapeutic agents to treat fibrotic diseases.

TLR9 inhibition has been recognized as a potential route to therapies for fibrotic diseases including idiopathic pulmonary fibrosis (Trujillo et al. Sci. Trans Med. 2010, 2(57):57ra82; Yoshizaki et al. Ann Rheum Dis. 2016 October; 75(10):1858-65), non-alcoholic steatohepatitis (Garcia-Martinez et al. J Clin invest 2016, 126: 859-864; Gabele et al. Biochem Biophys Res Commun. 2008; 376:271-276), hepatic injury (Shaker et al. Biochem Pharmacol. 2016. 112:90-101; Hoeque et al. J. Immun. 2013, 190:4297-304), and scleroderma (systemic sclerosis or SSc) (Yoshizaki et al. Ann Rheum Dis. 2016 October; 75(10):1858-65); as well as heart failure (Oka et al. Nature 485, pages 251-255(2012)), and hypertension (McCarthy et al. Cardiovascular Research, 2015, Pages 119-130).

There remains a need for compounds useful as inhibitors of TLR9. Additionally, there remains a need for compounds useful as inhibitors of TLR9 that have selectivity over TLR7 or TLR8.

In view of the conditions that may benefit by treatment involving modulation of Toll-like receptors, it is immediately apparent that new compounds capable of inhibiting TLR9 and methods of using these compounds could provide substantial therapeutic benefits to a wide variety of patients.

Applicants have found potent compounds that have activity as TLR9 inhibitors. Further, applicants have found compounds that have activity as TLR9 inhibitors and are selective over TLR7 or TLR8. These compounds are provided to be useful as pharmaceuticals with desirable stability, bioavailability, therapeutic index, and toxicity values that are important to their druggability.

### SUMMARY OF THE INVENTION

The present invention relates to a new class of substituted quinolone compounds found to be effective inhibitors of signaling through TLR9. These compounds are provided to be useful as pharmaceuticals with desirable stability, bioavailability, therapeutic index, and toxicity values that are important to their druggability.

The present invention provides compounds of Formula (I) that are useful as inhibitors of signaling through Toll-like receptor 9 and are useful for the treatment of fibrotic diseases, or stereoisomers, N-oxides, tautomers, pharmaceutically acceptable salts, solvates or prodrugs thereof.

The present invention also provides pharmaceutical compositions comprising a pharmaceutically acceptable carrier and at least one of the compounds of the present invention or stereoisomers, tautomers, pharmaceutically acceptable salts, solvates, or prodrugs thereof.

The present invention also provides a method for inhibition of Toll-like receptor 9 comprising administering to a host in need of such treatment a therapeutically effective amount of at least one of the compounds of the present invention or stereoisomers, tautomers, pharmaceutically acceptable salts, solvates, or prodrugs thereof.

The present invention also provides a method for treating fibrotic diseases, comprising administering to a host in need of such treatment a therapeutically effective amount of at least one of the compounds of the present invention or stereoisomers, tautomers, pharmaceutically acceptable salts, solvates, or prodrugs thereof.

The present invention also provides a method of treating a disease or disorder associated with Toll-like receptor 9 activity, the method comprising administering to a mammal in need thereof, at least one of the compounds of Formula (I) or salts, solvates, and prodrugs thereof.

The present invention also provides processes and intermediates for making the compounds of Formula (I) including salts, solvates, and prodrugs thereof.

The present invention also provides at least one of the compounds of Formula (I) or salts, solvates, and prodrugs thereof, for use in therapy.

The present invention also provides the use of at least one of the compounds of Formula (I) or salts, solvates, and prodrugs thereof, for the manufacture of a medicament for the treatment of prophylaxis of Toll-like receptor 9 related conditions, such as fibrotic diseases, autoimmune diseases, or inflammatory diseases.

The compound of Formula (I) and compositions comprising the compounds of Formula (I) may be used in treating, preventing, or curing various Toll-like receptor 9 related conditions. Pharmaceutical compositions comprising these compounds are useful for treating, preventing, or slowing the progression of diseases or disorders in a variety of therapeutic areas, such as fibrotic diseases including nonalcoholic steatohepatitis (NASH), non-alcoholic fatty liver disease (NAFLD), idiopathic pulmonary fibrosis, primary sclerosing cholangitis (PSC), and primary biliary cirrhosis (PBC).

These and other features of the invention will be set forth in expanded form as the disclosure continues.

### DETAILED DESCRIPTION

The first aspect of the present invention provides at least one compound of Formula (I):

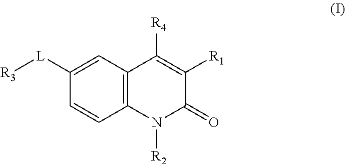

or a salt thereof wherein:
R₁ is phenyl, pyridinyl, pyrimidinyl, indolyl, indazolyl, or quinoxalinyl, each substituted with zero to 5 R₁ₐ;
each R₁ₐ is independently F, Cl, —CN, —OH, C₁₋₂ alkyl, C₁₋₂ fluoroalkyl, C₁₋₂ alkoxy, C₁₋₂ fluoroalkoxy, —C(O)OH, —C(O)O(C₁₋₂ alkyl), —C(O)NRₓRₓ, —NRₓRₓ, —NRₓC(O)(C₁₋₄ alkyl), —NHC(O)C(CH₃)₂OH—, —NHC(O)NRₓRₓ, —NO₂, —S(O)₂(C₁₋₂ alkyl), —S(O)₂NRₓRₓ, —NRₓS(O)₂(C₁₋₂ alkyl), —NRₓC(O)(pyridinyl), —NRₓ(methylazetidinyl), —C(O)(morpholinyl), —C(O)(dimethyl pyrrolidinyl), fluoropyrrolidinyl, hydroxypyrrolidinyl, pyrrolidinonyl, imidazolonyl, or tetrazolyl;
R₂ is hydrogen, C₁₋₄ alkyl, or C₁₋₄ hydroxyalkyl;
L is a bond or —NH—;
R₃ is phenyl or piperidinyl, each substituted with R₃ₐ;
R₃ₐ is —CH₃ or a cyclic group selected from piperidinyl and piperazinyl, wherein said cyclic group is substituted with zero or one R_{3b};
R_{3b} is C₁₋₃ alkyl or oxetanyl;
R₄ is hydrogen or C₁₋₂ alkyl; and
each Rₓ is independently H or —CH₃.

In one embodiment, a compound of Formula (I) or a salt thereof is provided wherein:
R₁ is phenyl, pyridinyl, pyrimidinyl, indolyl, indazolyl, or quinoxalinyl, each substituted with zero to 5 R₁ₐ;
each R₁ₐ is independently F, Cl, —CN, —OH, —CH₃, —CF₃, —OCH₃, —OCHF₂, —OCF₃, —C(O)OH, —C(O)OCH₃, —C(O)NH₂, —C(O)NH(CH₃), —C(O)N(CH₃)₂, —N(CH₃)₂, —NHC(O)CH₃, —NHC(O)CH₂CH₃, —NHC(O)CH(CH₃)₂, —NHC(O)CH(CH)₂CH₂CH₂CH₃, —NHC(O)C(CH₃)₂OH, —NHC(O)NH(CH₃), —NO₂, —S(O)₂CH₃, —S(O)₂NH₂, —NHS(O)₂CH₃, —N(CH₃)S(O)₂CH₃, —NHC(O)(pyridinyl), —C(O)N(CH₃)(oxetanyl), —N(CH₃)(methylazetidinyl), —C(O)(morpholinyl), —C(O)(dimethyl pyrrolidinyl), fluoropyrrolidinyl, hydroxypyrrolidinyl, pyrrolidinonyl, imidazolonyl, or tetrazolyl;
R₂ is hydrogen, —CH₃, —CH₂CH₃, —CH₂CH₂CH₃, —CH₂CH(CH₃)₂, —CH₂CH₂OH, —CH₂CH₂CH₂OH, or —CH₂C(CH₃)₂OH;
L is a bond or —NH—;
R₃ is phenyl or piperidinyl, each substituted with R₃ₐ;
R₃ₐ is —CH₃ or a cyclic group selected from piperidinyl aid piperazinyl, wherein said cyclic group is Substituted with zero or one R_{3b};
R_{3b} is —CH₃, —CH(CH₃)₃, or oxetanyl; and
R₄ is hydrogen or —CH₂CH₃.

In one embodiment, a compound of Formula (I), or a salt thereof is provided wherein R₁ is phenyl, pyridinyl, or pyrimidinyl, each substituted with zero to 5 R₁ₐ. Included in this embodiment are compounds in which R₁ is phenyl or pyridinyl, each substituted with zero to 5 R₁ₐ.

In one embodiment, a compound of Formula (I), or a salt thereof is provided wherein R₁ is phenyl substituted with zero to 5 R₁ₐ. Included in this embodiment are compounds in which R₁ is phenyl substituted with zero to 4 R₁ₐ. Also included in this embodiment are compounds in which R₁ is phenyl substituted with zero to 3 R₁ₐ.

In one embodiment, a compound of Formula (I), or a salt thereof is provided wherein R₁ is phenyl substituted with zero to 5 R₁ₐ; and each R₁ₐ is independently F, Cl, —CN, —CF₃, —OCH₃, —OCHF₂, —OCF₃, —C(O)OH, —C(O)OCH₃, —C(O)NH₂, —C(O)NH(CH₃), —C(O)N(CH₃)₂, —NHC(O)CH₃, —NHC(O)CH₂CH₃, —NHC(O)CH(CH₃)₂, —NHC(O)CH₂CH₂CH₂CH₃, —NHC(O)C(CH₃)₂OH, —NHC(O)NH(CH₃), —S(O)₂CH₃, —S(O)₂NH₂, —NHS(O)₂CH₃, or —N(CH₃)S(O)₂CH₃. Included in this embodiment are compounds in which R₁ is phenyl substituted with zero to 5 R₁ₐ; and each R₁ₐ is independently F, Cl, —CN, —CF₃, —OCH₃, —OCHF₂, —OCF₃, —NHC(O)CH₂CH₃, —S(O)₂CH₃, —S(O)₂NH₂, —NHS(O)₂CH₃, or —N(CH₃)S(O)₂CH₃.

In one embodiment, a compound of Formula (I) or a salt thereof is provided wherein each R₁ₐ is independently F, Cl, —CN, —OH, —CH₃, —CF₃, —OCH₃, —OCHF₂, —OCF₃, —C(O)OH, —C(O)OCH₃, —C(O)NH₂, —C(O)NH(CH₃), —C(O)N(CH₃)₂, —N(CH₃)₂, —NHC(O)CH₃, —NHC(O)CH₂CH₃, —NHC(O)CH(CH₃)₂, —NHC(O)CH₂CH₂CH₂CH₃, —NHC(O)C(CH₃)₂OH, —NHC(O)NH(CH₃), —NO₂, —S(O)₂CH₃, —S(O)₂NH₂, —NHS(O)₂CH₃, —N(CH₃)S(O)₂CH₃, —NHC(O)(pyridinyl), —C(O)N(CH₃)(oxetanyl), —N(CH₃)(Methylazetidin), —C(O)(morpholinyl), —C(O)(dimethyl pyrrolidinyl), fluoropyrrolidinyl, hydroxypyrrolidinyl, pyrrolidinonyl, imidazolonyl, or tetrazolyl. Included in this embodiment are compounds in which each R₁ₐ is independently F, Cl, —CN, —OH, —CH₃, —CF₃, —OCH₃, —OCHF₂, —OCF₃, —C(O)OH, —C(O)OCH₃, —C(O)NH₂, —C(O)NH(CH₃), —C(O)N(CH₃)₂, —N(CH₃)₂, —NHC(O)CH₃, —NHC(O)CH₂CH₃, —NHC(O)CH(CH₃)₂, —NHC(O)CH₂CH₂CH₂CH₃, —NHC(O)C(CH₃)₂OH, —NHC(O)NH(CH₃), —NO₂, —S(O)₂CH₃, —S(O)₂NH₂, —NHS(O)₂CH₃, or —N(CH₃)S(O)₂CH₃. Also included in this embodiment are compounds in which each R₁ₐ is independently F, Cl, —CN, —CH₃, —CF₃, —OCH₃, —OCHF₂, —OCF₃, —C(O)NH₂, —C(O)NH(CH₃), —C(O)N(CH₃)₂, —N(CH₃)₂, —NHC(O)CH₃, —NHC(O)CH₂CH₂CH₃, —NHC(O)CH(CH₃)₂, —NHC(O)CH₂CH₂CH₂CH₃, —NHC(O)C(CH₃)₂OH, —NHC(O)NH(CH₃), —NO₂, —S(O)₂CH₃, —S(O)₂NH₂, —NHS(O)₂CH₃, or —N(CH₃)S(O)₂(CH₃).

In one embodiment, a compound of Formula (I) or a salt thereof is provided wherein R₂ is C₁₋₄ alkyl or C₁₋₄ hydroxyalkyl. Included in this embodiment are compounds in which R₂ is C₁₋₄ alkyl. Also included in this embodiment are compounds in which R₂ is C₁₋₄ hydroxyalkyl.

In one embodiment, a compound of Formula (I) or a salt thereof is provided wherein R₂ is hydrogen.

In one embodiment, a compound of Formula (T) or a salt thereof is provided wherein R₂ is hydrogen, —CH₃, —CH₂CH₃, —CH₂CH₂CH₃, —CH₂CH(CH₃)₂, —CH₂CH₂OH, —CH₂CH₂CH₂OH, or —CH₂C(CH₃)₂OH. Included in this embodiment are compounds in which R₂ is hydrogen or —CH₃.

In one embodiment, a compound of Formula (I) or a salt there of is provided wherein L is a bond.

In one embodiment, a compound of Formula (I) or a salt there of is provided wherein L is —NH—.

In one embodiment, a compound of Formula (I) or a salt thereof is provided wherein R₃ is phenyl substituted with R₃ₐ.

In one embodiment, a compound of Formula (I) or a salt thereof is provided wherein R₃ is piperidinyl substituted with R₃ₐ.

In one embodiment, a compound of Formula (I) or a salt thereof is provided wherein R₃ₐ is —CH₃.

In one embodiment, a compound of Formula (I) or a salt there of is provided wherein R₃ₐ is a cyclic group selected from piperidinyl and piperazinyl, wherein said cyclic group is substituted with zero or one R_{3b}.

In one embodiment, a compound of Formula (I) or a salt thereof is provided wherein Ra is piperidinyl substituted with zero or one R_{3b}. Included in this embodiment are compounds in which R₃ₐ is piperidinyl substituted with zero R_{3b}. Also included in this embodiment are compounds in which R₃ₐ is piperidinyl substituted with R_{3b}.

In one embodiment, a compound of Formula (I) or a salt there of is provided wherein R₃ₐ is piperazinyl substituted with zero or one R_{3b}. Included in this embodiment are compounds in which R₃ₐ is piperazinyl substituted with zero R_{3b}. Also included in this embodiment are compounds in which R₃ₐ is piperazinyl substituted with R_{3b}.

In one embodiment, a compound of Formula (I) or a salt thereof is provided wherein R₃ₐ is piperidinyl or piperazinyl, each substituted with R_{3b}; and R_{3b} is —CH₃, —CH(CH₃)₃, or oxetanyl. Included in this embodiment are compounds in which R₃ₐ is piperidinyl or piperazinyl, each substituted with R_{3b}; and R_{3b} is —CH(CH₃)₃ or oxetanyl.

In one embodiment, a compound of Formula (I) or a salt thereof is provided wherein R₃ₐ is piperidinyl substituted with R_{3b}; and R_{3b} is —CH(CH₃)₃ or oxetanyl. Included in this embodiment are compounds in which R₃ₐ is piperidinyl substituted with R_{3b}; and R_{3b} is —CH(CH₃)₃.

In one embodiment, a compound of Formula (I) or a sat thereof is provided wherein R₃ₐ is piperazinyl substituted with R_{3b}; and R_{3b} is —CH(CH₃)₃ or oxetanyl. Included in this embodiment are compounds in which R₃ₐ is piperazinyl substituted with R_{3b}; and R_{3b} is —CH(CH₃)₃.

In one embodiment, a compound of Formula (I) or a salt thereof is provided wherein R₃ is:

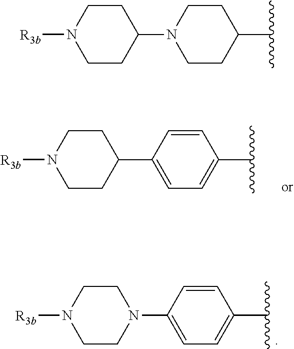

In one embodiment, a compound of Formula (I) or a salt thereof is provided wherein R₃ is:

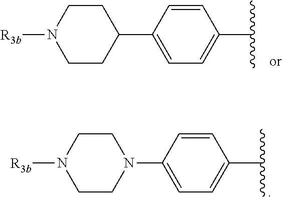

In one embodiment, a compound of Formula (I) or a salt there of is provided wherein R is piperidinyl substituted with R₃ₐ; and R₃ₐ is —CH₃.

In one embodiment, a compound of Formula (I) or a salt thereof is provided wherein R₄ is hydrogen or —CH₃CH₃.

In one embodiment, a compound of Formula (I) or a salt thereof is provided wherein R₄ is hydrogen.

In one embodiment, a compound of Formula (I) or a salt thereof is provided wherein R₁ is phenyl substituted with zero to 5 R₁ₐ; each R₁ₐ is independently F, Cl, —CN, —OH, —CH, —OCH₃, —OCHF₂, —OCF₃, —C(O)OH, —C(O)OCH₃, —C(O)NH₂, —C(O)NH(CH₃), —C(O)N(CH₃)₂, —N(CH₃)₂, —NHC(O)CH₃, —NHC(O)CH₂CH₃, —NHC(O)CH(CH₃)₂, —NHC(O)CH₂CH₂CH₂CH₃, —NHC(O)C(CH₃)₂OH, —NHC(O)NH(CH₃), —NO₂, —S(O)₂CH₃, —S(O)₂NH₂, —NHS(O)₂CH₃, —N(CH₃)S(O)₂CH₃, —NHC(O)(pyridinyl), —C(O)N(CH₃)(oxetanyl), —N(CH₃)(methylazetidinyl), —C(O)(morpholinyl), —C(O)(dimethyl pyrrolidinyl), fluoropyrrolidinyl, hydroxypyrrolidinyl, pyrrolidinonyl, imidazolonyl, or tetrazolyl; R₂ is hydrogen; L is a bond; R₃ is phenyl substituted with R₃ₐ; R₃ₐ is a cyclic group selected from piperidinyl and piperazinyl, wherein said cyclic group is substituted with zero or one R_{3b}; R_{3b} is —CH(CH₃)₃; and R₄ is hydrogen.

One embodiment provides a compound of Formula (I) or a salt thereof, wherein said compound is: 6-(4-(4-isopropylpiperazin-1-yl)phenyl)-1-methyl-3-(4-nitrophenyl) quinolin-2(1H)-one (1); 3-(4-hydroxyphenyl)-6-(4-(4-isopropylpiperazin-1-yl)phenyl) quinolin-2(1H)-one (2); 6-((4-(4-isopropylpiperazin-1-yl)phenyl)amino)-1-methyl-3-(4-(methylsulfonyl)phenyl)quinolin-2(1H)-one (3); 3-(4-methanesulfonylphenyl)-6-{4-[4-(propan-2-yl)piperazin-1-yl]phenyl}-1,2-dihydroquinolin-2-one (4); N-[4-(2-oxo-6-{4-[4-(propan-2-yl)piperazin-1-yl]phenyl}-1,2-dihydroquinolin-3-yl)phenyl]acetamide (5); 6-{4-[4-(propan-2-yl)piperazin-1-yl]phenyl}-3-(pyridin-2-yl)-1,2-dihydroquinolin-2-one (6); 3-(4-(1H-tetrazol-1-yl)phenyl)-6-(4-(4-isopropylpiperazin-1-yl)phenyl)quinolin-2(1H)-one (7); N-[3-(2-oxo-6-{4-[4-(propan-2-yl)piperazin-1-yl]phenyl}-1,2-dihydroquinolin-3-yl)phenyl]methanesulfonamide (8); 3-[4-(dimethylamino)phenyl]-6-{4-[4-(propan-2-yl)piperazin-1-yl]phenyl}-1,2-dihydroquinolin-2-one (9); 6-{4-[4-(propan-2-yl)piperazin-1-yl]phenyl}-3-(2,4,5-trifluorophenyl)-1,2-dihydroquinolin-2-one (10); 4-(2-oxo-6-{4-[4-(propan-2-yl)piperazin-1-yl]phenyl}-1,2-dihydroquinolin-3-yl) benzene-1-sulfonamide (11); 4-(2-oxo-6-{4-[4-(propan-2-yl)piperazin-1-yl]phenyl}-1,2-dihydroquinolin-3-yl)benzonitrile (12); N-methyl-N-(oxetan-3-yl)-4-(2-oxo-6-{4-[4-(propan-2-yl)piperazin-1-yl]phenyl}-1,2-dihydroquinolin-3-yl)benzamide (13); 3-[4-(2-oxoimdazolidin-1-yl)phenyl]-6-{4-[4-(propan-2-yl)piperazin-1-yl]phenyl}-1,2-dihydroquinolin-2-one (14); 2-(2-oxo-6-{4-[4-(propan-2-yl)piperazin-1-yl]phenyl}-1,2-dihydroquinolin-3-yl)benzonitrile (15); N-[4-(2-oxo-6-{4-[4-(propan-2-yl)piperazin-1-yl]phenyl}-1,2-dihydroquinolin-3-yl)phenyl]methanesulfonamide (16); 6-{4-[4-(propan-2-yl)piperazin-1-yl]phenyl}-3-(pyridin-4-yl)-1,2-dihydroquinolin-2-one (17); 3-phenyl-6-{4-[4-(propan-2-yl)piperazin-1-yl]phenyl}-1,2-dihydroquinolin-2-one (18); 3-[4-(2,2-dimethylpyrrolidine-1-carbonyl)phenyl]-6-{4-[4-(propan-2-yl)piperazin-1-yl]phenyl}-1,2-dihydroquinolin-2-one (19); 3-(2,5-difluorophenyl)-6-{4-[4-(propan-2-vi)piperazin-1-yl]phenyl}-1,2-dihydroquinolin-2-one (20); N-[4-(2-oxo-6-{4-[4-(propan-2-yl) piperazin-1-yl]phenyl}-1,2-dihydroquinolin-3-yl)phenyl]butanamide (21); 3-{4-[(3S)-3-fluoropyrrolidin-1-yl]phenyl}-6-{4-[4-(propan-2-yl)piperazin-1-yl]phenyl}-1,2-dihydroquinolin-2-one (22); N-methyl-N-[4-(1-methyl-2-oxo-6-{4-[4-(propan-2-yl) piperazin-1-yl]phenyl}-1,2-dihydroquinolin-3-yl)phenyl]methanesulfonamide (23); 3-(2,3-difluorophenyl)-6-{4-[4-(propan-2-yl)piperazin-1-yl]phenyl}-1,2-dihydroquinolin-2-one (24); 3-(2-methylphenyl)-6-{4-[4-(propan-2-yl)piperazin-1-yl]phenyl}-1,2-dihydroquinolin-2-one (25); 2-methyl-N-[4-(1-methyl-2-oxo-6-{4-[4-(propan-2-yl) piperazin-1-yl]phenyl}-1,2-dihydroquinolin-3-yl)phenyl]propanamide (26); 3-(4-methanesulfonylphenyl)-1-methyl-6-{4-[4-(propan-2-yl)piperazin-1-yl]phenyl}-1,2-dihydroquinolin-2-one (27); methyl 4-(2-oxo-6-{4-[4-(propan-2-yl)piperazin-1-yl]phenyl}-1,2-dihydroquinolin-3-yl)benzoate (28); N-[2-(2-oxo-6-{4-[4-(propan-2-yl) piperazin-1-yl]phenyl}-1,2-dihydroquinolin-3-yl)phenyl]methanesulfonamide (29); 3-(3-methoxyphenyl)-6-{4-[4-(propan-2-yl)piperazin-1-yl]phenyl}-1,2-dihydroquinolin-2-one (30); 3-(4-fluorophenyl)-6-{4-[1-(propan-2-yl)piperazin-1-yl]phenyl}-1,2-dihydroquinolin-2-one (31); 6-{4-[4-(propan-2-yl)piperazin-1-yl]phenyl}-3-(pyridin-3-yl)-1,2-dihydroquinolin-2-one (32); 6-{4-[4-(propan-2-yl)piperazin-1-yl]phenyl}-3-[2-(trifluoromethyl)phenyl]-1,2-dihydroquinolin-2-one (33); 6-{4-[4-(propan-2-yl)piperazin-1-yl]phenyl}-3-(pyrimidin-5-yl)-1,2-dihydroquinolin-2-one (34); 3-(1-methyl-1H-indazol-3-yl)-6-{4-[4-(propan-2-yl)piperazin-1-yl]phenyl}-1,2-dihydroquinolin-2-one (35); 3-[4-(difluoromethoxy)phenyl]-6-{4-[4-(propan-2-yl)piperazin-1-yl]phenyl}-1,2-dihydroquinolin-2-one (36); 3-(2,6-dichlorophenyl)-6-{4-[4-(propan-2-yl)piperazin-1-yl]phenyl-1,2-dihydroquinolin-2-one (37); 3-(2,4-dichlorophenyl)-6-{4-[4-(propan-2-yl) piperazin-1-yl]phenyl}-1,2-dihydroquinolin-2-one (38); 3-(4-chlorophenyl)-6-{4-[4-(propan-2-yl)piperazin-1-yl]phenyl}-1,2-dihydroquinolin-2-one (39); 3-(2,3,4,5,6-pentafluorophenyl)-6-{4-[4-(propan-2-yl)piperazin-1-yl]phenyl}-1,2-dihydroquinolin-2-one (40); 6-{4-[4-(propan-2-yl)piperazin-1-yl]phenyl}-3-(2,4,6-trifluorophenyl)-1,2-dihydroquinolin-2-one (41); 3-(2-fluorophenyl)-6-{4-[4-(propan-2-yl)piperazin-1-yl]phenyl}-1,2-dihydroquinolin-2-one (42); 3-(3,4-dimethoxyphenyl)-6-{4-[4-(propan-2-yl) piperazin-1-yl]phenyl}-1,2-dihydroquinolin-2-one (43); 3-(2-chlorophenyl)-6-{4-[4-(propan-2-yl)piperazin-1-yl]phenyl}-1,2-dihydroquinolin-2-one (44); 6-{4-[4-(propan-2-yl)piperazin-1-yl]phenyl}-3-[3-(trifluoromethyl)phenyl]-1,2-dihydroquinolin-2-one (45); 6-{4-[4-(propan-2-yl)piperazin-1-yl]phenyl}-3-[3-(trifluoromethoxy)phenyl]-1,2-dihydroquinolin-2-one (46); 3-(3-fluorophenyl)-6-{4-[4-(propan-2-yl)piperazin-1-yl]phenyl}-1,2-dihydroquinolin-2-one (47); 3-(1-methyl-1H-indol-3-yl)-6-(4-[4-(propan-2-yl)piperazin-1-yl]phenyl}-1,2-dihydroquinolin-2-one (48); 3-methyl-1-[4-(2-oxo-6-{4-[4-(propan-2-yl)piperazin-1-yl]phenyl}-1,2-dihydroquinolin-3-yl)phenyl]urea (49); N-[4-(1-methyl-2-oxo-6-{4-[4-(propan-2-yl)piperazin-1-yl]phenyl}-1,2-dihydroquinolin-3-yl) phenyl]pyridine-2-carboxamide (50); 3-{4-[methyl(1-methylazetidin-3-yl)amino]phenyl}-6-{4-[4-(propan-2-yl)piperazin-1-yl]phenyl}-1,2-dihydroquinolin-2-one (51); N-[4-(2-oxo-6-{4-[4-(propan-2-yl)piperazin-1-yl]phenyl}-1,2-dihydroquinolin-3-yl)phenyl]propanamide (52); 4-(2-oxo-6-{4-[4-(propan-2-yl)piperazin-1-yl]phenyl}-1,2-dihydroquinolin-3-yl)benzamide (53); N-methyl-4-(2-oxo-6-{4-[4-(propan-2-yl) piperazin-1-yl]phenyl}-1,2-dihydroquinolin-3-yl)benzamide (54); 3-[4-(morpholine-4-carbonyl)phenyl]-6-{4-[4-(propan-2-yl)piperazin-1-yl]phenyl}-1,2-dihydroquinolin-2-one (55); 3-[4-(2-oxopyrrolidin-1-yl)phenyl]-6-{4-[4-(propan-2-yl)piperazin-1-yl]phenyl}-1,2-dihydroquinolin-2-one (56); N-[4-(1-methyl-2-oxo-6-{4-[4-(propan-2-yl) piperazin-1-yl]phenyl}-1,2-dihydroquinolin-3-yl)phenyl]acetamide (57); 1-methyl-3-phenyl-6-{4-[4-(propan-2-yl)piperazin-1-yl]phenyl}-1,2-dihydroquinolin-2-one (58); 2-hydroxy-2-methyl-N-[4-(1-methyl-2-oxo-6-{4-[4-(propan-2-yl)piperazin-1-yl]phenyl}-1,2-dihydroquinolin-3-yl)phenyl]propanamide (59); N,N-dimethyl-4-(2-oxo-6-{4-[4-(propan-2-yl)piperazin-1-yl]phenyl}-1,2-dihydroquinolin-3-yl)benzamide (60); 3-{4-[(3R)-3-hydroxypyrrolidin-1-yl]phenyl}-6-{4-[4-(propan-2-yl)piperazin-1-yl]phenyl}-1,2-dihydroquinolin-2-one (61); 4-(2-oxo-6-{4-[4-(propan-2-yl)piperazin-1-yl]phenyl}-1,2-dihydroquinolin-3-yl)benzoic acid (62); 3-(3,4-dimethoxyphenyl)-4-ethyl-6-[1′-(propan-2-yl)-[1,4′-bipiperidine]-4-yl]-1,2-dihydroquinolin-2-one (63); 3-phenyl-6-[1′-(propan-2-yl)-[1,4′-bipiperidine]-4-yl]-1,2-dihydroquinolin-2-one (64); 4-(2-oxo-6-{4-[1-(propan-2-yl)piperidin-4-yl]phenyl}-1,2-dihydroquinolin-3-yl)benzene-1-sulfonamide (65); 4-ethyl-3-(4-methanesulfonylphenyl)-6-[1′-(propan-2-yl)-[1,4′-bipiperidine]-4-yl]-1,2-dihydroquinolin-2-one (66); N-[3-(2-oxo-6-{4-[1-(propan-2-yl)piperidin-4-yl]phenyl}-1,2-dihydroquinolin-3-yl)phenyl]methanesulfonamide (67); 3-(4-methanesulfonylphenyl)-1-methyl-6-{4-[1-(propan-2-yl)piperidin-4-yl]phenyl}-1,2-dihydroquinolin-2-one (68); 3-(2-oxo-6-{4-[1-(propan-2-yl)piperidin-4-yl]phenyl}-1,2-dihydroquinolin-3-yl)benzonitrile (69): N-(4-{6-[4-(1-methylpiperidin-4-yl)phenyl]-2-oxo-1,2-dihydroquinolin-3-yl}phenyl)methanesulfonamide (70); 3-(4-methanesulfonylphenyl)-6-{4-[1-(propan-2-yl)piperidin-4-yl]phenyl}-1,2-dihydroquinolin-2-one (71); 1-(2-hydroxyethyl)-3-(4-methanesulfonylphenyl)-6-{4-[1-(propan-2-yl)piperidin-4-yl]phenyl}-1,2-dihydroquinolin-2-one (72); 1-methyl-3-phenyl-6-{4-[1-(propan-2-yl)piperidin-4-yl]phenyl}-1,2-dihydroquinolin-2-one (73); N-methyl-N-[4-(1-methyl-2-oxo-6-{4-[1-(propan-2-yl)piperidin-4-yl]phenyl}-1,2-dihydroquinolin-3-yl)phenyl]methanesulfonamide (74); 3-(4-methanesulfonylphenyl)-1-methyl-6-({4-[1-(propan-2-yl)piperidin-4-yl]phenyl}amino)-1,2-dihydroquinolin-2-one (75); 3-(4-methanesulfonylphenyl)-6-[4-(1-methylpiperidin-4-yl)phenyl]-1,2-dihydroquinolin-2-one (76); 4-ethyl-3-(4-methanesulfonylphenyl)-1-methyl-6-{4-[4-(propan-2-yl)piperazin-1-yl]phenyl}-1,2-dihydroquinolin-2-one (77); 3-(4-methanesulfonylphenyl)-1-methyl-6-[4-(1-methylpiperidin-4-yl)phenyl]-1,2-dihydroquinolin-2-one (78); 1-ethyl-3-(4-methanesulfonylphenyl)-6-{4-[1-(propan-2-yl)piperidin-4-yl]phenyl}-1,2-dihydroquinolin-2-one (79); 3-(4-methanesulfonylphenyl)-6-{4-[1-(propan-2-yl) piperidin-4-yl]phenyl}-1-propyl-1,2-dihydroquinolin-2-one (80); N-[4-(2-oxo-6-{4-[1-(propan-2-yl)piperidin-4-yl]phenyl}-1,2-dihydroquinolin-3-yl)phenyl]acetamide (81); 1-(3-hydroxypropyl)-3-(4-methanesulfonylphenyl)-6-{4-[1-(propan-2-yl)piperidin-4-yl]phenyl}-1,2-dihydroquinolin-2-one (82): N-[4-(2-oxo-6-{4-[1-(propan-2-yl)piperidin-4-yl]phenyl}-1,2-dihydroquinolin-3-yl)phenyl]methanesulfonamide (83); 4-ethyl-3-(4-methanesulfonylphenyl)-6-{4-[4-(propan-2-yl)piperazin-1-yl]phenyl}-1,2-dihydroquinolin-2-one (84); 3-(4-methanesulfonylphenyl)-1-(2-methylpropyl)-6-{4-[1-(propan-2-yl)piperidin-4-yl]phenyl}-1,2-dihydroquinolin-2-one (85); 4-ethyl-3-(4-methanesulfonylphenyl)-1-methyl-6-{4-[1-(propan-2-yl)piperidin-4-yl]phenyl}-1,2-dihydroquinolin-2-one (86); 3-(4-methanesulfonylphenyl)-1-methyl-6-{4-[1-(oxetan-3-yl)piperidin-4-yl]phenyl}-1,2-dihydroquinolin-2-one (87); 3-[4-(dimethylamino)phenyl]-6-{4-[1-(propan-2-yl)piperidin-4-yl]phenyl}-1,2-dihydroquinolin-2-one (88); 1-(2-hydroxy-2-methylpropyl)-3-(4-methanesulfonylphenyl)-6-{4-[1-(propan-2-yl)piperidin-4-yl]phenyl}-1,2-dihydroquinolin-2-one (89); 3-(4-methanesulfonylphenyl)-6-({4-[4-(propan-2-yl)piperazin-1-yl]phenyl}amino)-1,2-dihydroquinolin-2-one (90); 3-[4-(difluoromethoxy)phenyl]-6-{4-[1-(oxetan-3-yl)piperidin-4-yl]phenyl}-1,2-dihydroquinolin-2-one (91); 4-(6-{4-[1-(oxetan-3-yl)piperidin-4-yl]phenyl}-2-oxo-1,2-dihydroquinolin-3-yl)benzene-1-sulfonamide (92); 6-{4-[1-(propan-2-yl)piperidin-4-yl]phenyl}-3-(quinoxalin-6yl)-1,2-dihydroquinolin-2-one (93); 6-{4-[1-(oxetan-3-yl) piperidin-4-yl]phenyl}-3-(quinoxalin-6-yl)-1,2-dihydroquinolin-2-one (94); 3-(3,4-dimethoxyphenyl)-4-ethyl-6-{4-[4-(propan-2-yl)piperazin-1-yl]phenyl}-1,2-dihydroquinolin-2-one (95); 3-[4-(dimethylamino)phenyl]-1-methyl-6-{4-[1-(propan-2-yl)piperidin-4-yl]phenyl}-1,2-dihydroquinolin-2-one (96); 3-(4-methanesulfonylphenyl)-6-(1-methylpiperidin-4-yl)-1,2-dihydroquinolin-2-one (97); N-[3-(6-{4-[1-(oxetan-3-yl) piperidin-4-yl]phenyl}-2-oxo-1,2-dihydroquinolin-3-yl)phenyl]methanesulfonamide (98); 3-(6-{4-[1-(oxetan-3-yl)piperidin-4-yl]phenyl}-2-oxo-1,2-dihydroquinolin-3-yl) benzonitrile (99); 1-methyl-6-{4-[1-(oxetan-3-yl)piperidin-4-yl]phenyl}-3-phenyl-1,2-dihydroquinolin-2-one (100); or 4-ethyl-3-(4-methanesulfonylphenyl)-1-methyl-6-{4-[1-(oxetan-3-yl)piperidin-4-yl]phenyl}-1,2-dihydroquinolin-2-one (101);

One embodiment provides compounds of the Formula (I) having TLR9 IC₅₀ values of ≤0.6 μM.

One embodiment provides compounds of the Formula (I) having TLR9 IC₅₀ values of ≤0.1 μM.

One embodiment provides compounds of the Formula (I) having TLR9 IC₅₀ values of ≤0.05 μM.

One embodiment provides compounds of the Formula (I) having TLR9 IC₅₀ values of ≤0.025 μM.

One embodiment provides compounds of the Formula (I) having TLR9 IC₅₀ values of ≤0.015 M.

One embodiment provides compounds of the Formula (I) having TLR9 IC₅₀ values of ≤0.01 μM.

In another embodiment, the present invention provides a composition comprising at least one of the compounds of the present invention, or a stereoisomer, a tautomer, or a pharmaceutically acceptable salt or a solvate thereof.

In another embodiment, the present invention provides a pharmaceutical composition comprising a pharmaceutically acceptable carrier and at least one of the compounds of the present invention or a stereoisomer, a tautomer, or a pharmaceutically acceptable salt or a solvate thereof.

In another embodiment, the present invention provides a pharmaceutical composition, comprising a pharmaceutically acceptable carrier and a therapeutically effective amount of at least one of the compounds of the present invention or a stereoisomer, a tautomer, or a pharmaceutically acceptable salt or a solvate thereof.

In another embodiment, the present invention provides a process for making a compound of the present invention.

In another embodiment, the present invention provides an intermediate for making a compound of the present invention.

In another embodiment, the present invention provides a pharmaceutical composition as defined above further comprising one or more additional therapeutic agents.

### Definitions

The features and advantages of the invention may be more readily understood by those of ordinary skill in the art upon reading the following detailed description. It is to be appreciated that certain features of the invention that are, for clarity reasons, described above and below in the context of separate embodiments, may also be combined to form a single embodiment. Conversely, various features of the invention that are, for brevity reasons, described in the context of a single embodiment, may also be combined so as to form sub-combinations thereof. Embodiments identified herein as exemplary or preferred are intended to be illustrative and not limiting.

Unless specifically stated otherwise herein, references made in the singular may also include the plural. For example, “a” and “an” may refer to either one, or one or more.

As used herein, the phase “compounds” refers to at least one compound. For example, a compound of Formula (I) includes a compound of Formula (I) and two or more compounds of Formula (I).

Unless otherwise indicated, any heteroatom with unsatisfied valences is assumed to have hydrogen atoms sufficient to satisfy the valences.

The definitions set forth herein take precedence over definitions set forth in any patent, patent application, and/or patent application publication incorporated herein by reference.

Listed below are definitions of various terms used to describe the present invention. These definitions apply to the terms as they are used throughout the specification (unless they are otherwise limited in specific instances) either individually or as part of a larger group.

Throughout the specification, groups and substituents thereof may be chosen by one skilled in the field to provide stable moieties and compounds.

In accordance with a convention used in the art,

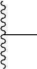

is used in structural formulas herein to depict the bond that is the point of attachment of the moiety or substituent to the core or backbone structure.

The terms “halo” and “halogen,” as used herein, refer to F, Cl, Br, and I.

The term “cyano” refers to the group —CN.

The term “amino” refers to the group —NH₂.

The term “oxo” refers to the group ═O.

The term “alkyl” as used herein, refers to both branched and straight-chain saturated aliphatic hydrocarbon groups containing, for example, from 1 to 12 carbon atoms, from 1 to 6 carbon atoms, and from 1 to 4 carbon atoms. Examples of alkyl groups include, but are not limited to, methyl (Me), ethyl (Et), propyl (e.g., n-propyl and i-propyl), butyl (e.g., n-butyl, i-butyl, sec-butyl, and t-butyl), and pentyl (e.g., n-pentyl, isopentyl, neopentyl), n-hexyl, 2-methylpentyl, 2-ethylbutyl, 3-methylpentyl, and 4-methylpentyl. When numbers appear in a subscript after the symbol “C” the subscript defines with more specificity the number of carbon atoms that a particular group may contain. For example, “C₁₋₆ alkyl” denotes straight and branched chain alkyl groups with one to six carbon atoms.

The term “fluoroalkyl” as used herein is intended to include both branched and straight-chain saturated aliphatic hydrocarbon groups substituted with one or more fluorine atoms. For example, “C₁₋₄ fluoroalkyl” is intended to include C₁, C₂, C₃, and C₄ alkyl groups substituted with one or more fluorine atoms. Representative examples of fluoroalkyl groups include, but are not limited to, —CF₃ and —CH₂CF₃.

The term “hydroxyalkyl” includes both branched and straight-chain saturated alkyl groups substituted with one or more hydroxyl groups. For example, “hydroxyalkyl” includes —CH₂OH, —CH₂CH₂OH, and C₁₋₄ hydroxyalkyl.

The term “alkoxy,” as used herein, refers to an alkyl group attached to the parent molecular moiety through an oxygen atom, for example, methoxy group (—OCH₃). For example, “C₁₋₃ alkoxy” denotes alkoxy groups with one to three carbon atoms.

The terms “fluoroalkoxy” and “—O(fluoroalkyl)” represent a fluoroalkyl group as defined above attached through an oxygen linkage (—O—). For example, “C₁₋₄ fluoroalkoxy” is intended to include C₁, C₂, C₃, and C₄ fluoroalkoxy groups.

The term “cycloalkyl,” as used herein, refers to a group derived from a non-aromatic monocyclic or polycyclic hydrocarbon molecule by removal of one hydrogen atom from a saturated ring carbon atom. Representative examples of cycloalkyl groups include, but are not limited to, cyclopropyl, cyclopentyl, and cyclohexyl. When numbers appear in a subscript after the symbol “C”, the subscript defines with more specificity the number of carbon atoms that a particular cycloalkyl group may contain. For example, “C₃₋₆ cycloalkyl” denotes cycloalkyl groups with three to six carbon atoms.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The compounds of Formula (I) can be provided as amorphous solids or crystalline solids. Lyophilization can be employed to provide the compounds of Formula (I) as amorphous solids.

It should further be understood that solvates (e.g., hydrates) of the compounds of Formula (I) are also within the scope of the present invention. The term “solvate” means a physical association of a compound of Formula (I) with one or more solvent molecules, whether organic or inorganic. This physical association includes hydrogen bonding. In certain instances the solvate will be capable of isolation, for example when one or more solvent molecules are incorporated in the crystal lattice of the crystalline solid. “Solvate” encompasses both solution-phase and isolable solvates. Exemplary solvates include hydrates, ethanolates, methanolates, isopropanolates, acetonitrile solvates, and ethyl acetate solvates. Methods of solvation are known in the art.

Various forms of prodrugs are well known in the art and are described in Rautio, J. et al., Nature Review Drug Discovery, 17, 559-587 (2018).

In addition, compounds of Formula (I), subsequent to their preparation, can be isolated and purified to obtain a composition containing an amount by weight equal to or greater than 99% of a compound of Formula (I), respectively (“substantially pure”), which is then used or formulated as described herein. Such “substantially pure” compounds of Formula (I) are also contemplated herein as part of the present invention.

“Stable compound” and “stable structure” are meant to indicate a compound that is sufficiently robust to survive isolation to a useful degree of purity from a reaction mixture, and formulation into an efficacious therapeutic agent. The present invention is intended to embody stable compounds.

“Therapeutically effective amount” is intended to include an amount of a compound of the present invention alone or an amount of the combination of compounds claimed or an amount of a compound of the present invention in combination with other active ingredients effective to act as an inhibitor of TLR9, or effective to treat or prevent disorders associated with a fibrotic disease or disorder, dysregulation of bile acids, such as pathological fibrosis.

As used herein, “treating” or “treatment” cover the treatment of a disease-state in a mammal, particularly in a human, and include: (a) preventing the disease-state from occurring in a mammal, in particular, when such mammal is predisposed to the disease-state but has not yet been diagnosed as having it; (b) inhibiting the disease-state, i.e., arresting its development; and/or (c) relieving the disease-state. i.e., causing regression of the disease state.

The compounds of the present invention are intended to include all isotopes of atoms occurring in the present compounds. Isotopes include those atoms having the same atomic number but different mass numbers. By way of general example and without limitation, isotopes of hydrogen include deuterium (D) and tritium (T). Isotopes of carbon include ¹³C and ¹⁴C. Isotopically-labeled compounds of the invention can generally be prepared by conventional techniques known to those skilled in the art or by processes analogous to those described herein, using an appropriate isotopically-labeled reagent in place of the non-labeled reagent otherwise employed. For example, methyl (—CH₃) also includes deuterated methyl groups such as —CD₃.

### Utility

The compounds of the invention are useful for inhibiting the TLR9 receptor.

One embodiment provides a method for the treatment of a disease, disorder, or condition associated with dysregulation of bile acids in a patient in need of such treatment, and the method comprises administering a therapeutically effective amount of a compound of the present invention, or a stereoisomer, a tautomer, or a pharmaceutically acceptable salt or solvate thereof, to the patient.

One embodiment provides a method for the treatment of a disease, disorder, or condition associated with activity of the TLR9 receptor in a patient in need of such treatment comprising administering a therapeutically effective amount of a compound of the present invention, or a stereoisomer, a tautomer, or a pharmaceutically acceptable salt or solvate thereof, to the patient.

One embodiment provides a method for the treatment of the disease, disorder, or condition comprising administering to a patient in need of such treatment a therapeutically effective amount of at least one of the compounds of the present invention, alone, or, optionally, in combination with another compound of the present invention and/or at least one other type of therapeutic agent.

One embodiment provides a method for eliciting an TLR9 receptor agonizing effect in a patient comprising administering a therapeutically effective amount of a compound of the present invention, or a stereoisomer, a tautomer, or a pharmaceutically acceptable salt or solvate thereof, to the patient.

In some embodiments, the disease, disorder, or condition is associated with TLR9 dysfunction include pathological fibrosis, cancer, inflammatory disorders, metabolic, or cholestatic disorders.

In some embodiments, the disease, disorder, or condition is associated with fibrosis, including liver, biliary, renal, cardiac, dermal, ocular, and pancreatic fibrosis.

In other embodiments, the disease, disorder, or condition is associated with cell-proliferative disorders, such as cancer. In some embodiments, the cancer includes solid tumor growth or neoplasia. In other embodiments, the cancer includes tumor metastasis. In some embodiments, the cancer is of the liver, gall bladder, small intestine, large intestine, kidney, prostate, bladder, blood, bone, brain, breast, central nervous system, cervix, colon, endometrium, esophagus, genitalia, genitourinary tract, head, larynx, lung, muscle tissue, neck, oral or nasal mucosa, ovary, pancreas, skin, spleen, stomach, testicle, or thyroid. In other embodiments, the cancer is a carcinoma, sarcoma, lymphoma, leukemia, melanoma, mesothelioma, multiple myeloma, or seminoma.

Examples of diseases, disorders, or conditions associated with the activity of TLR9 that can be prevented, modulated, or treated according to the present invention include, but are not limited to, transplant injection, fibrotic disorders (e, g., liver fibrosis, kidney fibrosis), inflammatory disorders (e.g., acute hepatitis, chronic hepatitis, non-alcoholic steatohepatitis (NASH), irritable bowel syndrome (IBS), inflammatory bowel disease (IBD)), as well as cell-proliferative disorders (e.g., cancer, myeloma, fibroma, hepatocellular carcinoma, colorectal cancer, prostate cancer, leukemia, Kaposi's sarcoma, solid tumors).

The fibrotic disorders, inflammatory disorders, as well as cell-proliferative disorders that are suitable to be prevented or treated by the compounds of the present invention include, but are not limited to, non-alcoholic fatty liver disease (NAFLD), alcoholic or non-alcoholic steatohepatitis (NASH), acute hepatitis, chronic hepatitis, liver cirrhosis, primary biliary cirrhosis, primary sclerosing cholangitis, drug-induced hepatitis, biliary cirrhosis, portal hypertension, regenerative failure, liver hypofunction, hepatic blood flow disorder, nephropathy, irritable bowel syndrome (IBS), inflammatory bowel disease (IBD), abnormal pancreatic secretion, benign prostatic hyperplasia, neuropathic bladder disease, diabetic nephropathy, focal segmental glomerulosclerosis, IgA nephropathy, nephropathy induced by drugs or transplantation, autoimmune nephropathy, lupus nephritis, liver fibrosis, kidney fibrosis, chronic kidney disease (CKD), diabetic kidney disease (DKD), skin fibrosis, keloids, systemic sclerosis, scleroderma, virally-induced fibrosis, idiopathic pulmonary fibrosis (IPF), interstitial lung disease, non-specific interstitial pneumonia (NSIP), usual interstitial pneumonia (UIP), radiation-induced fibrosis, familial pulmonary fibrosis, airway fibrosis, chronic obstructive pulmonary disease (COPD), spinal cord tumor, hernia of intervertebral disk, spinal canal stenosis, heart failure, cardiac fibrosis, vascular fibrosis, perivascular fibrosis, foot-and-mouth disease, cancer, myeloma, fibroma, hepatocellular carcinoma, colorectal cancer, prostate cancer, leukemia, chronic lymphocytic leukemia, Kaposi's sarcoma, solid tumors, cerebral infarction, cerebral hemorrhage, neuropathic pain, peripheral neuropathy, age-related macular degeneration (AMD), glaucoma, ocular fibrosis, corneal scarring, diabetic retinopathy, proliferative vitreoretinopathy (PVR), cicatricial pemphigoid glaucoma filtration surgery scarring, Crohn's disease or systemic lupus erythematosus; keloid formation resulting from abnormal wound healing: fibrosis occurring after organ transplantation, myelofibrosis, and fibroids. In one embodiment, the present invention provides a method for the treatment of a fibrotic disorder, an inflammatory disorder, or a cell-proliferative disorder, comprising administering to a patient in need of such treatment a therapeutically effective amount of at least one of the compounds of the present invention, alone, or, optionally, in combination with another compound of the present invention and/or at least one other type of therapeutic agent.

In another embodiment, the present invention provides a compound of the present invention for use in therapy.

In another embodiment, the present invention provides a compound of the present invention for use in therapy for the treatment of a fibrotic disorder, an inflammatory disorder, or a cell-proliferative disorder thereof.

In another embodiment, the present invention also provides the use of a compound of the present invention for the manufacture of a medicament for the treatment of a fibrotic disorder, an inflammatory disorder, or a cell-proliferative disorder thereof.

In another embodiment, the present invention provides a method for the treatment of a fibrotic disorder, an inflammatory disorder, or a cell-proliferative disorder, comprising administering to a patient in need thereof a therapeutically effective amount of a first and second therapeutic agent, wherein the first therapeutic agent is a compound of the present invention.

In another embodiment, the present invention provides a combined preparation of a compound of the present invention and additional therapeutic agent(s) for simultaneous, separate or sequential use in therapy.

In another embodiment, the present invention provides a combined preparation of a compound of the present invention and additional therapeutic agent(s) for simultaneous, separate or sequential use in the treatment of a fibrotic disorder, an inflammatory disorder, or a cell-proliferative disorder.

The compounds of the present invention may be employed in combination with additional therapeutic agent(s), such as one or more anti-fibrotic and/or anti-inflammatory therapeutic agents.

In one embodiment, additional therapeutic agent(s) used in combined pharmaceutical compositions or combined methods or combined uses, are selected from one or more, preferably one to three, of the following therapeutic agents: TGFβ receptor inhibitors (for example, galunisertib), inhibitors of TGFβ synthesis (for example, pirfenidone), inhibitors of vascular endothelial growth factor (VEGF), platelet-derived growth factor (PDCF) and fibroblast growth factor (FGF) receptor kinases (for example, nintedanib), humanized anti-αvβ6 integrin monoclonal antibody (for example, 3G9) human recombinant pentraxin-2, recombinant human Serum Amyloid P, recombinant human antibody against TGFβ-1, -2, and -3, endothelin receptor antagonists (for example, macitentan), interferon gamma, c-Jun amino-terminal kinase (JNK) inhibitor (for example, 4-[[9-[(3S)-tetrahydro-3-furanyl]-8-[(2,4,6-trifluorophenyl)amino]-9H-purin-2-yl]amino]-trans-cyclohexanol, 3-pentylbenzeneacetic acid (PBI-4050), tetra-substituted porphyrin derivative containing manganese (III), monoclonal antibody targeting eotaxin-2, interleukin-I3 (IL-13) antibody (for example, lebrikizumab, tralokinumab), bispecific antibody targeting interleukin 4 (IL-4) and interleukin 13 (TL-13), NK1 tachykinin receptor agonist (for example, Sar⁰, Met(O₂)¹¹-Substance P), Cintredekin Besudotox, human recombinant DNA-derived, IgG1 kappa monoclonal antibody to connective growth factor, and fully human igG1 kappa antibody, selective for CC-chemokine ligand 2 (for example, carlumab, CCX140), antioxidants (for example, N-acetylcysteine), phosphodiesterase 5 (PDE5) inhibitors (for example, sildenafil), agents for treatment of obstructive airway diseases such as muscarinic antagonists (for example, tiotropium, ipatropium bromide), adrenergic β2 agonists (for example, salbutamol, salmeterol), corticosteroids (for example, tilamcinolone, dexamethasone, fluticasone), immunosuppressive agents (for example, tacrolimus, rapamycin, pimecrolimus), and therapeutic agents useful for the treatment of fibrotic conditions, such as liver, biliary, and kidney fibrosis, Non-Alcoholic Fatty Liver Disease (NALFD), Non-Alcoholic Steato-Hepatitis (NASH), cardiac fibrosis, Idiopathic Pulmonary Fibrosis (IPF), and systemic sclerosis. The therapeutic agents useful for the treatment of such fibrotic conditions include, but are not limited to, FXR agonists (for example OCA, GS-9674, and LJN452), LOXL2 inhibitors (for example simtuzumab), LPA1 antagonists (for example, BMS-986020 and SAR 100842), PPAR modulators (for example, elafibrinor, pioglitazone, and saroglitazar, IVA337), SSAO/VAP-1 inhibitors (for example, PXS-4728A and SZE5302), ASK-1 inhibitors (for example GS-4997 or selonsertib), ACC inhibitors (for example, CP-640186 and NDI-010976 or GS-0976), FGF21 mimetics (for example, LY2405319 and BMS-986036), caspase inhibitors (for example, emricasan), NOX4 inhibitors (for example, GKT137831), MGAT2 inhibitor (for example, BMS-963272), αV integrin inhibitors (for example, abituzumab) and bile acid/fatty acid conjugates (for example aramchol). The TLR9 inhibitors of various embodiments of the present invention may also be used in combination with one or more therapeutic agents such as CCR2/5 inhibitors (for example, cenicriviroc), Galectin-3 inhibitors (for example, TD-139, GR-MD-02), leukotriene receptor antagonists (for example, tipelukast, montelukast), SGLT2 inhibitors (for example, dapagliflozin, remogliflozin), GLP-1 receptor agonists (for example, liraglutide and semaglutide), FAK inhibitors (for example, GSK-2256098), CB1 inverse agonists (for example, JD-5037), CB2 agonists (for example, APD-371 and JBT-101), autotaxin inhibitors (for example, GLPG1690), prolyl t-RNA synthetase inhibitors (for example, halofuginone), FPR2 agonists (for example, ZK-994), and THR agonists (for example, MGL: 3196). In another embodiment, additional therapeutic agent(s) used in combined pharmaceutical compositions or combined methods or combined uses, are selected from one or more, preferably one to three, of immunooncology agents, such as Alemtuzumab, Atezolizumab, Ipilimumab, Nivolumab, Ofatumumab, Pembrolizumab, and Rituximab.

When the terms “TLR9-associated condition” or “TLR9-associated disease or disorder” are used herein, each is intended to encompass all of the conditions identified above as if repeated at length, as well as any other condition that is affected by inhibition of TLR9.

The above other therapeutic agents, when employed in combination with the compounds of the present invention, may be used, for example, in those amounts indicated in the Physicians' Desk Reference (PDR) or as otherwise determined by one of ordinary skill in the art. In the methods of the present invention, such other therapeutic agent(s) may be administered prior to, simultaneously with, or following the administration of the inventive compounds. The present invention also provides pharmaceutical compositions capable of treating; TLR9-associated conditions.

The inventive compositions may contain other therapeutic agents as described above and may be formulated, for example, by employing conventional solid or liquid vehicles or diluents, as well as pharmaceutical additives of a type appropriate to the mode of desired administration (e.g., excipients, binders, preservatives, stabilizers, flavors, etc.) according to techniques such as those well known in the art of pharmaceutical formulation.

Accordingly, the present invention further includes compositions comprising one or more compounds of Formula (I) and a pharmaceutically acceptable carrier.

A “pharmaceutically acceptable carrier” refers to media generally accepted in the art for the delivery of biologically active agents to animals, in particular, mammals. Pharmaceutically acceptable carriers are formulated according to a number of factors well within the purview of those of ordinary skill in the art. These include without limitation the type and nature of the active agent being formulated: the subject to which the agent-containing composition is to be administered: the intended route of administration of the composition; and, the therapeutic indication being targeted. Pharmaceutically acceptable carriers include both aqueous and non-aqueous liquid media, as well as a variety of solid and semi-solid dosage forms. Such carriers can include a number of different ingredients and additives in addition to the active agent, such additional ingredients being included in the formulation for a variety of reasons, e.g., stabilization of the active agent, binders, etc., well known to those of ordinary skill in the art. Descriptions of suitable pharmaceutically acceptable carriers, and factors involved in their selection, are found in a variety of readily available sources such as, for example, Remington's Pharmaceutical Sciences, 17th Edition (1985), which is incorporated herein by reference in its entirety.

Compounds in accordance with Formula (I) can be administered by any means suitable for the condition to be treated, which can depend on the need for site-specific treatment or quantity of Formula (I) compound to be delivered.

Also embraced within this invention is a class of pharmaceutical compositions comprising a compound of Formula (I), and one or more non-toxic, pharmaceutically-acceptable carriers and/or diluents and/or adjuvants (collectively referred to herein as “carrier” materials) and, if desired, other active ingredients. The compounds of Formula (I) may be administered by any suitable route, preferably in the form of a pharmaceutical composition adapted to such a route, and in a dose effective for the treatment intended. The compounds and compositions of the present invention may, for example, be administered orally, mucosally, or parentally including intravascularly, intravenously, intraperitoneally, subcutaneously, intramuscularly, and intrasternally in dosage unit formulations containing conventional pharmaceutically acceptable carriers, adjuvants, and vehicles. For example, the pharmaceutical carrier may contain a mixture of mannitol or lactose and microcrystalline cellulose. The mixture may contain additional components such as a lubricating agent, e.g. magnesium stearate and a disintegrating agent such as crospovidone. The carrier mixture may be filled into a gelatin capsule or compressed as a tablet. The pharmaceutical composition may be administered as an oral dosage form or an infusion, for example.

For oral administration, the pharmaceutical composition may be in the form of, for example, a tablet, capsule, liquid capsule, suspension, or liquid. The pharmaceutical composition is preferably made in the form of a dosage unit containing a particular amount of the active ingredient. For example, the pharmaceutical composition may be provided as a tablet or capsule comprising an amount of active ingredient in the range of from about 0.1 to 1000 mg, preferably from about 0.25 to 250 mg, and more preferably from about 0.5 to 100 mg. A suitable daily dose for a human or other mammal may vary widely depending on the condition of the patient and other factors, but, can be determined using routine methods.

Any pharmaceutical composition contemplated herein can, for example, be delivered orally via any acceptable and suitable oral preparations. Exemplary oral preparations, include, but are not limited to, for example, tablets, troches, lozenges, aqueous and oily suspensions, dispersible powders or granules, emulsions, hard and soft capsules, liquid capsules, syrups, and elixirs. Pharmaceutical compositions intended for oral administration can be prepared according to any methods known in the art for manufacturing pharmaceutical compositions intended for oral administration. In order to provide pharmaceutically palatable preparations, a pharmaceutical composition in accordance with the invention can contain at least one agent selected from sweetening agents, flavoring agents, coloring agents, demulcents, antioxidants, and preserving agents.

A tablet can, for example, be prepared by admixing at least one compound of Formula (I) with at least one non-toxic pharmaceutically acceptable excipient suitable for the manufacture of tablets. Exemplary excipients include, but are not limited to, for example, inert diluents, such as, for example, calcium carbonate, sodium carbonate, lactose, calcium phosphate, and sodium phosphate; granulating and disintegrating agents, such as, for example, microcrystalline cellulose, sodium crosscarmellose, corn starch, and alginic acid; binding agents, such as, for example, starch, gelatin, polyvinyl-pyrrolidone, and acacia; and lubricating agents, such as, for example, magnesium stearate, stearic acid, and talc. Additionally, a tablet can either be uncoated, or coated by known techniques to either mask the bad taste of an unpleasant tasting drug, or delay disintegration and absorption of the active ingredient in the gastrointestinal tract thereby sustaining the effects of the active ingredient for a longer period. Exemplary water soluble taste masking materials, include, but are not limited to, hydroxypropyl-methylcellulose and hydroxypropyl-cellulose. Exemplary time delay materials, include, but are not limited to, ethyl cellulose and cellulose acetate butyrate.

Hard gelatin capsules can for example, be prepared by mixing at least one compound of Formula (I) with at least one inert solid diluent, such as, for example, calcium carbonate; calcium phosphate; and kaolin.

Soft gelatin capsules can, for example, be prepared by mixing at least one compound of Formula (I) with at least one water soluble carrier, such as, for example, polyethylene glycol; and at least one oil medium, such as, for example, peanut oil, liquid paraffin, and olive oil.

An aqueous suspension can be prepared, for example, by admixing at least one compound of Formula (I) with at least one excipient suitable for the manufacture of an aqueous suspension. Exemplary excipients suitable for the manufacture of an aqueous suspension, include, but are not limited to, for example, suspending agents, such as, for example, sodium carboxymethylcellulose, methylcelluloses, hydroxypropylmethyl-cellulose, sodium alginate, alginic acid, polyvinyl-pyrrolidone, gum tragacanth, and gum acacia; dispersing or wetting agents, such as, for example, a naturally-occurring phosphatide, e.g., lecithin; condensation products of alkylene oxide with fatty acids, such as, for example, polyoxyethylene stearate; condensation products of ethylene oxide with long chain aliphatic alcohols, such as, for example heptadecaethylene-oxycetanol; condensation products of ethylene oxide with partial esters derived from fatty acids and hexitol, such as, for example, polyoxyethylene sorbitol monooleate; and condensation products of ethylene oxide with partial esters derived from fatty acids and hexitol anhydrides, such as, for example, polyethylene sorbitan monooleate. An aqueous suspension can also contain at least one preservative, such as, for example, ethyl and n-propyl p-hydroxybenzoate; at least one coloring agent; at least one flavoring agent; and/or at least one sweetening agent, including but not limited to, for example, sucrose, saccharin, and aspartame.

Oily suspensions can, for example, be prepared by suspending at least one compound of Formula (I) in either a vegetable oil, such as, for example, arachis oil; olive oil; sesame oil; and coconut oil; or in mineral oil, such as, for example, liquid paraffin. An oily suspension can also contain at least one thickening agent, such as, for example, beeswax; hard paraffin; and cetyl alcohol. In order to provide a palatable oily suspension, at least one of the sweetening agents already described hereinabove, and/or at least one flavoring agent can be added to the oily suspension. An oily suspension can further contain at least one preservative, including, but not limited to, for example, an antioxidant, such as, for example, butylated hydroxyanisol, and alpha-tocopherol.

Dispersible powders and granules can, for example, be prepared by admixing at least one compound of Formula (I) with at least one dispersing and/or wetting agent; at least one suspending agent; and/or at least one preservative. Suitable dispersing agents, wetting agents, and suspending agents are as already described above. Exemplary preservatives include, but are not limited to, for example, anti-oxidants, e.g., ascorbic acid. In addition, dispersible powders and granules can also contain at least one excipient, including, but not limited to, for example, sweetening agents; flavoring agents; and coloring agents.

An emulsion of at least one compound of Formula (I) thereof can, for example, be prepared as an oil-in-water emulsion. The oily phase of the emulsions comprising compounds of Formula (I) may be constituted from known ingredients in a known manner. The oil phase can be provided by, but is not limited to, for example, a vegetable oil, such as, for example, olive oil and arachis oil; a mineral oil, such as, for example, liquid paraffin; and mixtures thereof. While the phase may comprise merely an emulsifier, it may comprise a mixture of at least one emulsifier with a fat or an oil or with both a fat and an oil. Suitable emulsifying agents include, but are not limited to, for example, naturally-occurring phosphatides, e.g., soy bean lecithin; esters or partial esters derived from fatty acids and hexitol anhydrides, such as, for example, sorbitan monooleate; and condensation products of partial esters with ethylene oxide, such as, for example, polyoxyethylene sorbitan monooleate. Preferably, a hydrophilic emulsifier is included together with a lipophilic emulsifier which acts as a stabilizer. It is also preferred to include both an oil and a fat. Together, the emulsifier(s) with or without stabilizer(s) make-up the so-called emulsifying wax, and the wax together with the oil and fat make up the so-called emulsifying ointment base which forms the oily dispersed phase of the cream formulations. An emulsion can also contain a sweetening agent, a flavoring agent, a preservative, and/or an antioxidant. Emulsifiers and emulsion stabilizers suitable for use in the formulation of the present invention include Tween 60, Span 80, cetostearyl alcohol, myristyl alcohol, glyceryl monostearate, sodium lauryl sulfate, glyceryl distearate alone or with a wax, or other materials well known in the art.

The compounds of Formula (I) can, for example, also be delivered intravenously, subcutaneously, and/or intramuscularly via any pharmaceutically acceptable and suitable injectable form. Exemplary injectable forms include, but are not limited to, for example, sterile aqueous solutions comprising acceptable vehicles and solvents, such as, for example, water, Ringer's solution, and isotonic sodium chloride solution; sterile oil-in-water microemulsions; and aqueous or oleaginous suspensions.

Formulations for parenteral administration may be in the form of aqueous or non-aqueous isotonic sterile injection solutions or suspensions. These solutions and suspensions may be prepared from sterile powders or granules using one or more of the carriers or diluents mentioned for use in the formulations for oral administration or by using other suitable dispersing or wetting agents and suspending agents. The compounds may be dissolved in water, polyethylene glycol, propylene glycol, ethanol, corn oil, cottonseed oil, peanut oil, sesame oil, benzyl alcohol, sodium chloride, tragacanth gum, and/or various buffers. Other adjuvants and modes of administration are well and widely known in the pharmaceutical art. The active ingredient may also be administered by injection as a composition with suitable carriers including saline, dextrose, or water, or with cyclodextrin (i.e. Captisol), cosolvent solubilization (i.e. propylene glycol) or micellar solubilization (i.e. Tween 80).

The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed, including synthetic mono- or diglycerides. In addition, fatty acids such as oleic acid find use in the preparation of injectables.

A sterile injectable oil-in-water microemulsion can, for example, be prepared by 1) dissolving at least one compound of Formula (I) in an oily phase, such as, for example, a mixture of soybean oil and lecithin; 2) combining the Formula (I) containing oil phase with a water and glycerol mixture; and 3) processing the combination to form a microemulsion.

A sterile aqueous or oleaginous suspension can be prepared in accordance with methods already known in the art. For example, a sterile aqueous solution or suspension can be prepared with a non-toxic parenterally-acceptable diluent or solvent, such as, for example, 1,3-butane diol; and a sterile oleaginous suspension can be prepared with a sterile non-toxic acceptable solvent or suspending medium, such as, for example, sterile fixed oils, e.g., synthetic mono- or diglycerides; and fatty acids, such as, for example, oleic acid.

Pharmaceutically acceptable carriers, adjuvants, and vehicles that may be used in the pharmaceutical compositions of this invention include, but are not limited to, ion exchangers, alumina, aluminum stearate, lecithin, self-emulsifying drug delivery systems (SEDDS) such as d-alpha-tocopherol polyethyleneglycol 1000 succinate, surfactants used in pharmaceutical dosage forms such as Tweens, polyethoxylated castor oil such as CREMOPHOR surfactant (BASF), or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as alpha-, beta-, and gamma-cyclodextrin, or chemically modified derivatives such as hydroxyalkylcyclodextrins, including 2- and 3-hydroxypropyl-cyclodextrins, or other solubilized derivatives may also be advantageously used to enhance delivery of compounds of the formulae described herein.

The pharmaceutically active compounds of this invention can be processed in accordance with conventional methods of pharmacy to produce medicinal agents for administration to patients, including humans and other mammals. The pharmaceutical compositions may be subjected to conventional pharmaceutical operations such as sterilization and/or may contain conventional adjuvants, such as preservatives, stabilizers, wetting agents, emulsifiers, buffers etc. Tablets and pills can additionally be prepared with enteric coatings. Such compositions may also comprise adjuvants, such as wetting, sweetening, flavoring, and perfuming agents.

The amounts of compounds that are administered and the dosage regimen for treating a disease condition with the compounds and/or compositions of this invention depends on a variety of factors, including the age, weight, sex, the medical condition of the subject, the type of disease, the severity of the disease, the route and frequency of administration, and the particular compound employed. Thus, the dosage regimen may vary widely, but can be determined routinely using standard methods. A daily dose of about 0.001 to 100 mg/kg body weight, preferably between about 0.0025 and about 50 mg/kg body weight and most preferably between about 0.005 to 10 mg/kg body weight, may be appropriate. The daily dose can be administered in one to four doses per day. Other dosing schedules include one dose per week and one dose per two day cycle.

For therapeutic purposes, the active compounds of this invention are ordinarily combined with one or more adjuvants appropriate to the indicated route of administration. If administered orally, the compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as may be provided in a dispersion of active compound in hydroxypropylmethyl cellulose.

Pharmaceutical compositions of this invention comprise at least one compound of Formula (I) and optionally an additional agent selected from any pharmaceutically acceptable carrier, adjuvant, and vehicle. Alternate compositions of this invention comprise a compound of the Formula (I) described herein, or a prodrug thereof, and a pharmaceutically acceptable carrier, adjuvant, or vehicle.

The present invention also encompasses an article of manufacture. As used herein, article of manufacture is intended to include, but not be limited to, kits and packages. The article of manufacture of the present invention, comprises: (a) a first container; (b) a pharmaceutical composition located within the first container, wherein the composition, comprises: a first therapeutic agent, comprising: a compound of the present invention or a pharmaceutically acceptable salt form thereof; and, (c) a package insert stating that the pharmaceutical composition can be used for the treatment of a cardiovascular disorder, diuresis, and/or natriuresis. In another embodiment, the package insert states that the pharmaceutical composition can be used in combination (as defined previously) with a second therapeutic agent to treat cardiovascular disorder, diuresis, and/or natriuresis. The article of manufacture can further comprise: (d) a second container, wherein components (a) and (b) are located within the second container and component (c) is located within or outside of the second container. Located within the first and second containers means that the respective container holds the item within its boundaries.

The first container is a receptacle used to hold a pharmaceutical composition. This container can be for manufacturing, storing, shipping, and/or individual/bulk selling. First container is intended to cover a bottle, jar, vial, flask, syringe, tube (e.g., for a cream preparation), or any other container used to manufacture, hold, store, or distribute a pharmaceutical product.

The second container is one used to hold the first container and, optionally, the package insert. Examples of the second container include, but are not limited to, boxes (e.g., cardboard or plastic), crates, cartons, bags (e.g., paper or plastic bags), pouches, and sacks. The package insert can be physically attached to the outside of the first container via tape, glue, staple, or another method of attachment, or it can rest inside the second container without any physical means of attachment to the first container. Alternatively, the package insert is located on the outside of the second container. When located on the outside of the second container, it is preferable that the package insert is physically attached via tape, glue, staple, or another method of attachment. Alternatively, it can be adjacent to or touching the outside of the second container without being physically attached.

The package insert is a label, tag, marker, or other written sheet that recites information relating to the pharmaceutical composition located within the first container. The information recited will usually be determined by the regulatory agency governing the area in which the article of manufacture is to be sold (e.g., the United States Food and Drug Administration). Preferably, the package insert specifically recites the indications for which the pharmaceutical composition has been approved. The package insert may be made of any material on which a person can read information contained therein or thereon. Preferably, the package insert is a printable material (e.g., paper, plastic, cardboard, foil, adhesive-backed paper or plastic) on which the desired information has been formed (e.g., printed or applied).

### Methods of Preparation

The compounds of the present invention can be prepared in a number of ways well known to one skilled in the art of organic synthesis. The compounds of the present invention can be synthesized using the methods described below, together with synthetic methods known in the art of synthetic organic chemistry, or variations thereon as appreciated by those skilled in the art. Preferred methods include, but are not limited to, those described below.

The reactions and techniques described in this section are performed in solvents appropriate to the reagents and materials employed and are suitable for the transformations being effected. Also, in the description of the synthetic methods described below, it is to be understood that all proposed reaction conditions, including choice of solvent, reaction atmosphere, reaction temperature, duration of the experiment and work up procedures, are chosen to be the conditions standard for that reaction, which should be readily recognized by one skilled in the art. It is understood by one skilled in the art of organic synthesis that the functionality present on various portions of the molecule must be compatible with the reagents and reactions proposed. Such restrictions to the substituents that are compatible with the reaction conditions will be readily apparent to one skilled in the art and alternate methods must then be used. This will sometimes require a judgment to modify the order of the synthetic steps or to select one particular process scheme over another in order to obtain a desired compound of the invention. It will also be recognized that another major consideration in the planning of any synthetic route in this field is the judicious choice of the protecting group used for protection of the reactive functional groups present in the compounds described in this invention. An authoritative account describing the many alternatives to the trained practitioner is Greene et al. (Protective Groups in Organic Synthesis, Third Edition, Wiley and Sons (1999)).

### EXAMPLES

Compounds of the current invention and intermediates used in the preparation of compounds of the current invention can be prepared using procedures shown in the following examples and related procedures. The methods and conditions used in these examples, and the actual compounds prepared in these examples, are not meant to be limiting, but are meant to demonstrate how the compounds of the current invention can be prepared. Starting materials and reagents used in these examples, when not prepared by a procedure described herein, are generally either commercially available, or are reported in the chemical literature, or may be prepared by using procedures described in the chemical literature. The invention is further defined in the following Examples, it should be understood that the Examples are given by way of illustration only. From the above discussion and the Examples, one skilled in the art can ascertain the essential characteristics of the invention, and without departing from the spirit and scope thereof, can make various changes and modifications to adapt the invention to various uses and conditions. As a result, the invention is not limited by the illustrative examples set forth herein below, but rather defined by the claims appended hereto.

In the examples given, the phrase “dried and concentrated” generally refers to drying of a solution in an organic solvent over either sodium sulfate or magnesium sulfate, followed by filtration and removal of the solvent from the filtrate (generally under reduced pressure and at a temperature suitable to the stability of the material being Chemical names were determined using ChemDraw Professional, version 20.1.0.110 (PerkinElmer Informatics, Inc.). The following abbreviations are used:
AA acetic acid
ACN acetonitrile
AcOH acetic acid
aq. aqueous
brine saturated aqueous sodium chloride
DCM dichloromethane
DIPEA diisopropylethylamnine
DMF N,N-dimethylformamide
DMSO dimethyl sulfoxide
EDCI 1-(3-dimethylaminopropyl)-3-ethylcarbodiimide hydrochloride
EtOAc ethyl acetate
EtOH ethanol
g grain(s)
h hour(s)
HATU O-(7-azabenzotriazol-1-yl)-N,N,N′,N-tetramethyluronium hexafluorophosphate
HPLC High Performance Liquid Chromatography
IPA isopropyl alcohol
LCMS Liquid Chromatography-Mass Spectroscopy
min minute(s)
MeCN acetonitrile
MeOH methanol
NaOtBu sodium tertiary butoxide
Pd(dba)₃ tris-(dibenzylideneacetone)dipalladium
pet ether petroleum ether
T3P solution propylphosphonic anhydride solution
tBUBrettPhos Pd G3 [2′-(Amino-kappaN)[1,1′-biphenyl]-2-yl-kappaC][[3,6-dimethoxy-2′,4′,6′-tris(1-methylethyl)[1,1′-biphenyl]-2-ditert-butyl-[3,6-dimethoxy-2-[2,4,6-tri(propan-2-yl)phenyl]phenyl]phosphane; methanesulfonic acid; palladium; 2-phenylaniline
TEA triethylamine
TFA trifluoroacetic acid
THF tetrahydrofuran
Xantphos 4,5-bis(diphenylphosphino)-9,9 dimethylxanthene
Xphos Pd G2 chloro(2-dicyclohexylphosphino-2′,4′,6′-triisopropyl-1,1′-biphenyl)[2-(2′-amino-1,1′-biphenyl)]palladium(II)

### General Scheme for the Synthesis of Piperazinyl Quinolones

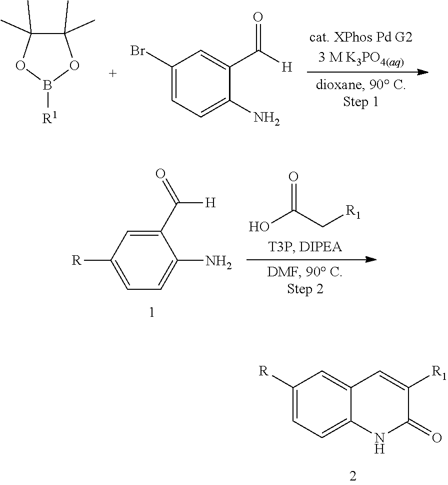

### Step 1. General Procedure for the Preparation of Compound 1:

To a glass vial was added 2-amino-5-bromobenzaldehyde (1.0 equiv.) 1-isopropyl-4-(4-(4,4,5,5-tetramethyl-1,3,2-dioxaborolan-2-yl)phenyl)piperazine (1.0 equiv.), XPhos Pd G2 (0.1 equiv.), and 3 M K₃PO₄ (3.0 equiv.). The vial was sparged with N₂ and a vent needle. Next, 1,4-dioxane (0.2 M) was added and the mixture was heated to 90° C. for 1 hour. The mixture was loaded onto a Celite precolumn and subjected to flash column chromatography with DCM/MeOH as the eluent. Product-containing fractions were combined and concentrated under reduced pressure to give compound 1.

### Synthesis of 4-amino-4′-(4-isoproylpiperazin-1-yl)-[1,1′-biphenyl]-3-carbaldehyde

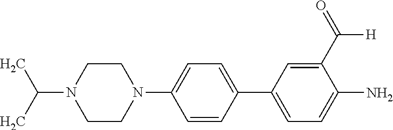 [SMILES: CC(C)N1CCN(c2ccc(-c3ccc(N)c(C=O)c3)cc2)CC1]

To a 40 mL vial was added 2-amino-5-bromobenzaldehyde (0.65 g, 3.2 mmol) 1-isopropyl-4-(4-(4,4,5,5-tetramethyl-1,3,2-dioxaborolan-2-yl)phenyl)piperazine (1.1 g, 3.2 mmol), XPhos Pd G2 (0.26 g, 0.32 mmol), and 3 M K₃PO₄ (3.2 mL, 9.7 mmol). The vial was sparged with N₂ and a vent needle. Next, 1,4-dioxane (11 mL) was added and the mixture was heated to 90° C. for 1 hour. The mixture was loaded onto a Celite precolumn and subjected to flash column chromatography with DCM/MeOH as the eluent. Product-containing fractions were combined and concentrated under reduced pressure to give 4-amino-4′-(4-isopropylpiperazin-1-yl)-[1,1′-biphenyl]-3-carbaldehyde (929 mg, 2.9 mmol, 89% yield) as a green solid, Analytical Method 3: Observed mass: 324.2 (MH⁺); Retention Time: 1.1 min. Analytical Method 4 m/z 324.2 (MH⁺) Retention Time: 1.30 min. ¹H NMR (500 MHz, CHLOROFORM-d) δ 9.94 (s, 1H), 7.66 (d, J=2.2 Hz, 1H), 7.55 (dd, J:=8.5, 2.2 Hz, 1H), 7.45 (d, J=8.7 Hz, 2H), 6.99 (d, J=8.9 Hz, 2H), 6.71 (d, J=8.6 Hz, 1H), 6.09 (br s, 2H), 3.28-3.21 (m, 4H), 2.74-2.67 (m, 5H), 1.10 (d, J=6.5 Hz, 6H).

### Step 2. General Procedure for the Formation of R₁ Substituted Quinolones (Compound 2):

To a 4 mL vial was added 4-amino-4′-(4-isopropylpiperazin-1-yl)-[1,1′-biphenyl]-3-carbaldehyde (1.0 equiv.), T3P solution in 50% DMF (1.5 equiv.), phenylacetic acid derivative (1.0 equiv.), DIPEA (2.0 equiv.), and DMF (0.2 M). The mixture was heated to 90° C. for 16 hours. Then the mixture was filtered through 0.45 μm syringe filter and purified by preparative HPLC. The product containing fractions were combined and concentrated by centrifugal evaporation to give the RI substituted quinolone (compound 2)

### General Scheme for the Synthesis of N-Alkylated Quinolones (5) and O-Alkylated Quinolines (6)

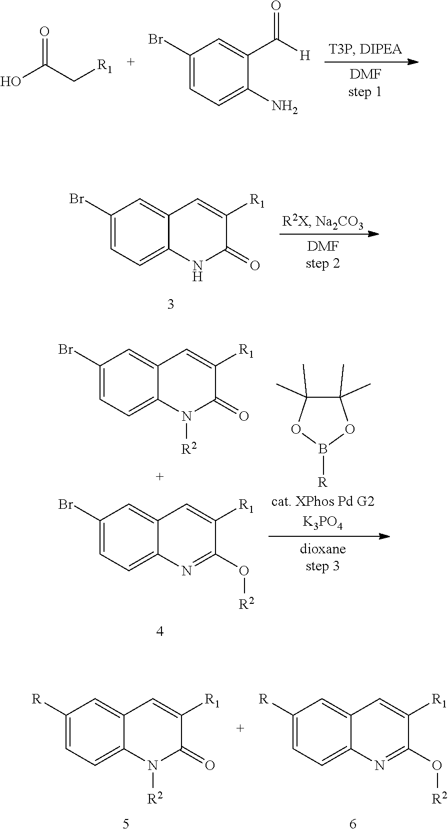

Step 1. General procedure for the formation of 6-bromoquinolones (3):

To a glass vial was added 2-amino-5-bromobenzaldehyde (1.0 equiv.), phenylacetic acid derivative (1.0 equiv.), T3P solution in 50% DMF (1.5 equiv.) DIPEA (2.0 equiv.), and DMF (0.2 M). The mixture was heated to 65-90° C. for 16 hours. The mixture was diluted with at least 50 mL water and solid precipitated. The solid was collected by filtration to afford 6-bromoquinolones (3).

### Example of 6-bromoquinolone (3): Synthesis of 6-bromo-3-(4-nitrophenyl)quinolin-2(1H)-one

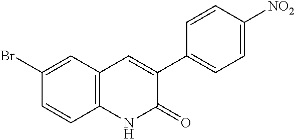 [SMILES: O=c1[nH]c2ccc(Br)cc2cc1-c1ccc([N+](=O)[O-])cc1]

To a 10 mL glass vial was added 2-amino-5-bromobenzaldehyde (500 mg, 2.5 mmol), 2-(4-nitrophenyl)acetic acid (450 mg, 2.5 mmol), T3P solution in 50% DMF (2.2 mL, 3.8 mmol), DIPEA (0.9 ml, 5.0 mmol), and DMF (12 mL). The mixture was heated to 65° C. for 16 hours. The mixture was diluted with at least 50 mL water and solid precipitated. The solid was collected by filtration to afford 6-bromo-3-(4-nitrophenyl) quinolin-2(1H)-one (810 μg, 2.3 mmol, 94% yield). ¹H NMR (500 MHz, DMSO-d₆) δ 8.36-8.29 (m, 2H), 8.09-8.04 (m, 2H), 8.04-7.97 (m, 1H), 7.75-7.68 (m, 1H), 7.37-7.26 (m, 1H).

### Step 2. General Procedure for the Formation of N-Alkylated Bromoquinolones (4):

To a glass vial was added 6-bromoquinolone (3) (1.0 equiv.), alkyl halide (3.0 equiv.), carbonate base (3.0 equiv.), and DMF (0.2 M). The mixture was heated to 70° C. for 10 min to 1 hour. The mixture was diluted with 5 volumes of distilled water and solid precipitated. The solid was collected by filtration to afford a mixture of N- and O-alkylated products.

### Example of N-alkylated Bromoquinolones (4): Synthesis of 6-bromo-1-methyl-3-(4-nitrophenyl)quinolin-2(1H)-one

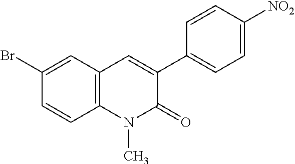 [SMILES: Cn1c(=O)c(-c2ccc([N+](=O)[O-])cc2)cc2cc(Br)ccc21]

To a 20 mL glass vial was added 6-bromo-3-(4-nitrophenyl)quinolin-2(1H)-one (390 mg, 1.1 mmol), iodomethane (0.2 mL, 3.4 mmol), cesium carbonate (1.1 g, 3.4 mmol), and DMF (5.7 mL). The mixture was heated to 70° C. for 10 min. The mixture was diluted with 5 volumes of distilled water and solid precipitated. The solid was collected by filtration to afford a mixture (409 mg, 1.1 mmol, 100% yield) of 6-bromo-1-methyl-3-(4-nitrophenyl)quinolin-2(1H)-one (Analytical Method 1: Observed mass: 358.8 (MH⁺); Retention Time: 1.09 min) and 6-bromo-2-methoxy-3-(4-nitrophenyl)quinoline (Analytical Method 2: Observed mass: 358.8 (MH⁺); Retention Time: 1.18 min).

### Step 3. General Procedure for the Formation of Compound 5:

To a glass vial was added aryl boronate ester (1.1 equiv.), bromoquinolone (1.0 equiv.), XPhos Pd G2 (0.1 equiv), and 3 M K₃PO₄ (3.0 equiv.). The mixture was sparged with N₂ and a vent needle for 5 minutes, dioxane (0.2 M) was added, and the mixture was heated to 90° C. for 20 min to 1 hour. The crude reaction mixture was subjected to normal phase flash column chromatography. Product containing fractions were combined and concentrated under reduced pressure to afford the Suzuki-coupling adduct.

### Example of Compound 5: Synthesis of 6-(4-(4-isopropylpiperazin-1-yl)phenyl)-1-methyl-3-(4-nitrophenyl)quinolin-2(1H)-one: (Example 1)

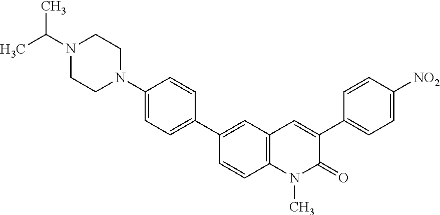 [SMILES: CC(C)N1CCN(c2ccc(-c3ccc4c(c3)cc(-c3ccc([N+](=O)[O-])cc3)c(=O)n4C)cc2)CC1]

To a 4 mL glass vial was added 1-isopropyl-4-(4-(4,4,5,5-tetramethyl-1,3,2-dioxaborolan-2-yl)phenyl)piperazine (198 mg, 0.60 mmol), 6-bromo-1-methyl-3-(4-nitrophenyl)quinolin-2(1H-one (196 mg, 0.50 mmol), XPhos Pd G2 (43 mg, 0.055 mmol), and 3 M K₃PO₄ (0.55 mL, 1.6 mmol). The mixture was sparged with N₂ and a vent needle and heated to 90° C. for 20 min. The crude reaction mixture was subjected to flash column chromatography with DCM/MeOH as the eluent. The product containing fractions were combined and concentrated under reduced pressure to afford 6-(4-(4-isopropylpiperazin-1-yl)phenyl)-1-methyl-3-(4-nitrophenyl)quinolin-2(1H)-one (263 mg, 0.55 mmol, 100% yield). Analytical Method 1: Observed mass: 483.3 (MH⁺); Retention Time: 0.848 min.

### General Scheme for Reduction of Nitroarene to Aniline

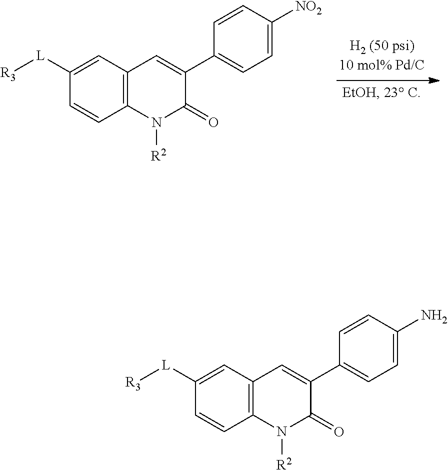

To a heavy-walled round-bottomed flask equipped with magnetic stirrer was added, under N₂ nitroarene (1 equiv.), wet Pd/C (0.1 equiv.), and ethanol (0.2 M). The flask was evacuated and backfilled with N₂ three times. Then, the flask was evacuated and backfilled with H₂ (50 psi) and stirred at 23° C. for 1 h. The reaction mixture was filtered through Celite and concentrated under reduced pressure to afford the aniline.

### General Scheme Preparation of Amide Derivatives 7

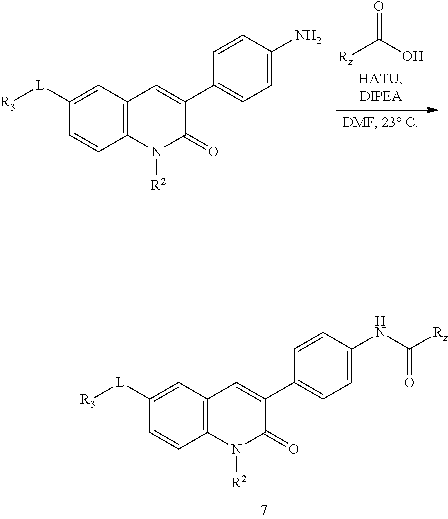

To a glass vial equipped with magnetic stirrer was added carboxylic acid (1.5 equiv,), HATU (1.5 equiv.) DMF (0.2 M), and DIPEA (2 equiv.) and the mixture was stirred at 23° C. for 2 min. The aniline compound (1 equiv.) was added and the mixture was stirred for 1 h at 23° C. The mixture was purified by preparative reverse phase HPLC to afford carboxamides 7.

### General Scheme for Amide Derivatives 9

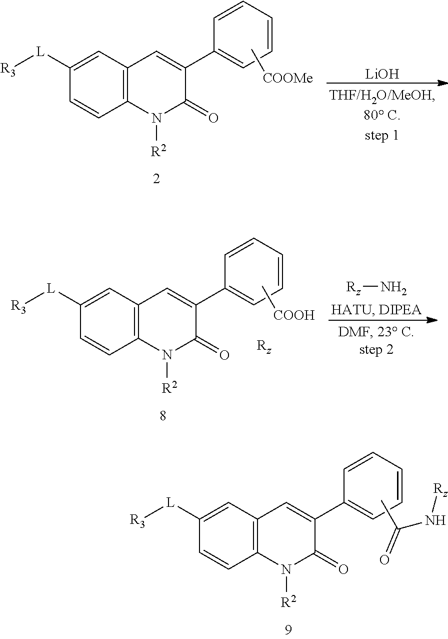

### Step 1. General Procedure for the Formation of Benzoic Acids (8):

To a glass vial was added benzoate ester (1.0 equiv.), 1 M lithium hydroxide solution (1.5 equiv.), and THF/water/MeOH (1/1/5, 0.1 M). The reaction mixture was heated to 80° C. for 1 h. The reaction mixture was dried by centrifugal evaporation to afford benzoic acid 8 as the lithium salt which was used directly in the amide bond coupling step.

### Step 2. General Procedure for the Formation of Amide Derivatives (9):

To a glass vial equipped with magnetic stirrer was added carboxylic acid (1.0 equiv.), 50% T3P in DMF (2.0 equiv.), DMF (0.1 M), and DIPEA (3 equiv.). The mixture was stirred at 23° C. for 2 min. The aniline (1.2 equiv.) was added and the mixture was stirred for 1 h at 23° C. The mixture was purified by preparative reverse phase HPLC to afford carboxamides 9.

### General Scheme for Aniline Derivatives 11

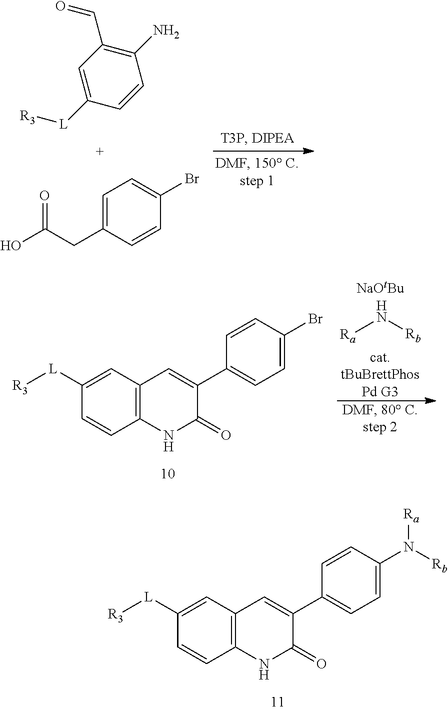

### Step 1. General Procedure for the Formation of Bromobenzene 10:

To a glass vial was added 2-aminoaldehyde (1.0 equiv), 2-(4-bromophenyl)acetic acid (1.2 equiv.), 50% T3P in DMF (1.5 equiv.), DIPEA (4.0 equiv.), and DMF (0.2 M). The mixture was heated by microwave to 150° C. for 1 h. The reaction mixture was quenched with 5×volumes 10% saturated LiCl and extracted with 3×equal volume EtOAc or DCM+5% MeOH. The organic layers were combined, dried over sodium sulfate, and concentrated under reduced pressure to afford bromobenzene 10.

### Step 2. General Procedure for the Formation of Aniline Derivatives 11:

To a glass vial equipped with magnetic stirrer was added bromobenzene 10 (1.0 equiv.), amine (2.0 equiv.), sodium tert-butoxide (3 equiv.), tBuBrettPhos Pd G3 (0.1 equiv.), and DMF (0.1 M). The mixture was sparged with N₂ and a vent needle for 5 minutes. The mixture was heated at 80° C. for 1 h. Si-thiol resin was added (0.5 equiv., 1.0 mmol/g), the solution was stirred for 30 min at 23° C. and passed through a 0.45 μm syringe filter. The mixture was purified by preparative reverse phase HPLC to afford aniline derivatives 11.

### Procedure for the Formation of 3-(4-hydroxyphenyl)-6-{4-[4-(propan-2-yl)piperazin-1-yl]phenyl}-1,2-dihydroquinolin-2-one: (Example 2)

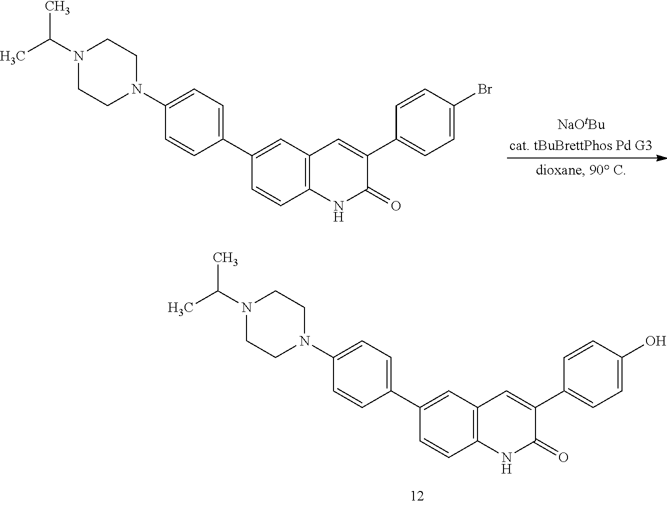 [SMILES: CC(C)N1CCN(c2ccc(-c3ccc4[nH]c(=O)c(-c5ccc(Br)cc5)cc4c3)cc2)CC1 ; CC(C)N1CCN(c2ccc(-c3ccc4[nH]c(=O)c(-c5ccc(O)cc5)cc4c3)cc2)CC1]

To a glass vial equipped with magnetic stirrer was added 3-(4-bromophenyl)-6-(4-(4-isopropylpiperazin-1-yl)phenyl)quinolin-2(1H)-one (30 mg, 0.060 mmol), sodium tert-butoxide (17.2 mg, 0.18 mmol), tBuBrettPhos Pd G3 (5.1 mg, 6.0 μmol), and dioxane (1 mL). The mixture was sparged with N₂ and a vent needle for 5 minutes. The mixture was heated at 90° C. for 3 h. The mixture was concentrated by centrifugal evaporation and purified by preparative reverse phase HPLC (Prep Method 1) to afford 3-(4-hydroxyphenyl)-6-(4-(4-isopropylpiperazin-1-yl)phenyl)quinolin-2(1H)-one (12) (3.1 mug, 0.0070 mmol, 12% yield). Analytical Method 5: Purity: 100%; Observed mass: 440.2 (MH⁺); Retention Time: 1.2 min. Analytical Method 6: Purity: 100%; Observed mass: 440.0 (MH⁺); Retention Time: 1.58 min. ¹H NMR (500 MHz, DMSO-d₆) δ 1.87 (s, 1H), 9.64 (s, 1H), 8.04 (s, 1H), 7.94 (s, 1H), 7.74 (dd, J=8.5, 1.7 Hz, 1H), 7.63 (d, J=8.6 Hz, 2H), 7.60 (br d, J=8.5 Hz, 2H), 7.36 (d, J=8.5 Hz, 1H), 7.07 (br d, J=8.2 Hz, 2H), 6.82. (d, J=8.5 Hz, 2H), 3.28-2.88 (m, 8H), 1.18 (br d, J=2.7 Hz, 7H).

### General Scheme for the Synthesis of C4-Alkylated Intermediate 15

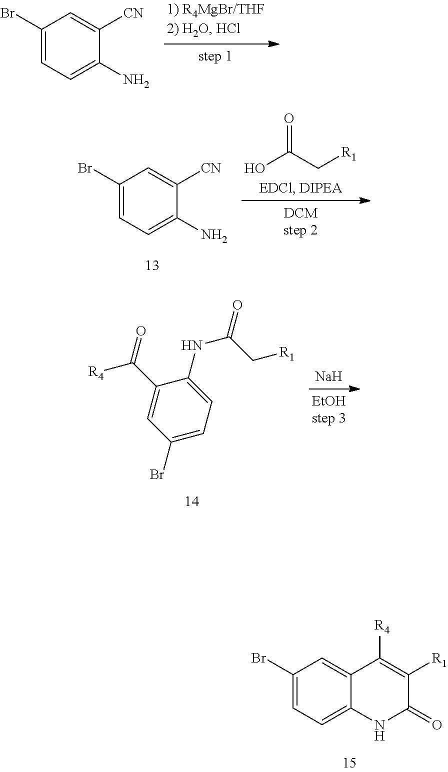

### Step 1. General Procedure for the Formation of Ketone 13:

To a glass vial equipped with magnetic stirrer was added 2-amino-5-bromobenzonitrile (1.0 equiv.) and THF (0.5 M). The mixture was cooled to 0° C. Next, 1.0 M Grignard solution in THE (3.0 equiv.) was added dropwise and the solution was stirred for 20 min at 0° C. and then warmed to 23° C. for 16 h. The solution was cooled to 0° C. The reaction was quenched with 6 M HCl (5.0 equiv.). The solution was stirred for 1 h., neutralized the solution with 1 N NaOH, and extracted with EtOAc. The organic layer was dried over sodium sulfate, filtered, and concentrated under reduced pressure to afford ketone 13.

### Example of Ketone 13: Synthesis of 1-(2-amino-5-bromophenyl)propan-1-one

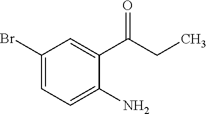

To a 20 mL glass vial equipped with magnetic stirrer was added 2-amino-5-bromobenzonitrile (2.1 g, 10.5 mmol) and THF (21 mL). The mixture was cooled to 0° C. Next, 1.0 M Grignard solution in THF (32 mL, 31.5 mmol) was added dropwise and the solution was stirred for 20 min at 0° C. and then warmed to 23° C. for 16 h. The solution was cooled 0° C. The reaction was quenched with 6 M HCl (8.8 mL, 53 mmol). The solution was stirred for 1 h., neutralized the solution with 1 N NaOH (100 mL), and extracted with EtOAc (100 mL). The organic layer was dried over sodium sulfate, filtered, and concentrated under reduced pressure to afford 1-(2-amino-5-bromophenyl) propan-1-one (2.35 g, 10.3 mmol, 98% yield). Analytical Method 1: Observed mass: 229.8 (MH⁺); Retention Time: 0.967 min. ¹H NMR (500 MHz, CHLOROFORM-d) δ 7.84 (d, J=2.2 Hz, 1H), 7.32 (dd, J=8.8, 2.3 Hz, 1H), 6.56 (d, J=8.9 Hz, 1H), 6.29 (br s, 2H), 2.95 (q, J=7.2 Hz, 2H), 1.21 (t, J=7.3 Hz, 3H).

### Step 2. General Procedure for the Formation of Ketoamide 14:

To a glass vial equipped with magnetic stirrer was added 13 (1.0 equiv.). EDCI (2.0 equiv.), phenylacetic acid (2.0 equiv.), DIPEA (3.0 equiv.), and DCM (0.2 M). The mixture was stirred at 23° C. for 2 h. The reaction mixture was subjected to flash column chromatography and product-containing fractions were concentrated under reduced pressure to afford ketoamide 14.

### Example of Ketoamide 14: Synthesis of N-(4-bromo-2-propionylphenyl)-2-(4-(methylsulfonyl)phenyl)acetamide

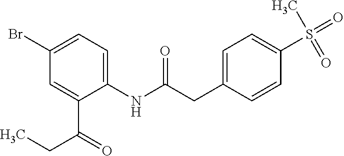 [SMILES: CCC(=O)c1cc(Br)ccc1NC(=O)Cc1ccc(S(C)(=O)=O)cc1]

To a 40 mL glass vial equipped with magnetic stirrer was was added 1-(2-amino-5-bromophenyl)propan-1-one (500 mg, 2.2 mmol), EDCI (840 mg, 4.4 mmol), phenylacetic acid (940 mg, 4.4 mmol), DIPEA (1.1 mL, 6.6 mmol), and DCM (11 mL). The mixture was stirred at 23° C. for 2 h. The reaction mixture was subjected to flash column chromatography and the product-containing fractions were concentrated under reduced pressure to afford N-(4-bromo-2-propionylphenyl)-2-(4-(methylsulfonyl)phenyl) acetamide (560 mg, 1.3 mmol, 60% yield). Analytical Method 1: Observed mass: 425.9 (MH⁺). Retention Time: 0.940 min.

### Step 3. General Procedure for the Formation of Quinolones 15:

To a glass vial equipped with magnetic stirrer was added ketoamide 14 (1.0 equiv.) and EtOH (02 M). The mixture was cooled to 0° C. To the stirring mixture was added 60% sodium hydride (3.0 equiv.) and after 5 min., the mixture was stirred for 1 h at 23° C. The solvent was evaporated by a stream of N₂ and water (5 volumes) was added to dissolve residual sodium ethoxide. The solid precipitate was filtered through sintered glass funnel and air dried to collect quinolones 15.

### Example of Quinolones 14: Synthesis of 6-bromo-4-ethyl-3-(4-(methylsulfonyl)phenyl) quinolin-2(1H)-one

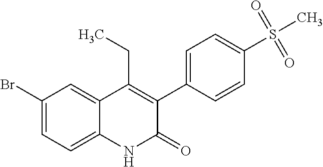 [SMILES: CCc1c(-c2ccc(S(C)(=O)=O)cc2)c(=O)[nH]c2ccc(Br)cc12]

To a 20 mL glass vial equipped with magnetic stirrer was added N-(4-bromo-2-propionylphenyl)-2-(4-(methylsulfonyl)phenyl)acetamide (558 mg, 1.3 mmol) and EtOH (6.6 mL). The reaction mixture was cooled to 0° C. To the stirring mixture was added 60% sodium hydride (158 mg, 4.0 mmol) and after 5 min., the mixture was stirred for 1 h at 23° C. The solvent was evaporated by a stream of N₂ and water (33 mL) was added to dissolve residual sodium ethoxide. The solid precipitate was filtered through sintered glass funnel and air dried to collect 6-bromo-4-ethyl-3-(4-(methylsulfonyl)phenyl) quinolin-2(1H)-one (480 mg, 1.2 mmol, 90% yield). Analytical Method 3: Observed mass: 407.8 (MH⁺); Retention Time: 1.712 min.

### General Scheme for the Synthesis of Bis Piperidine Compounds 18

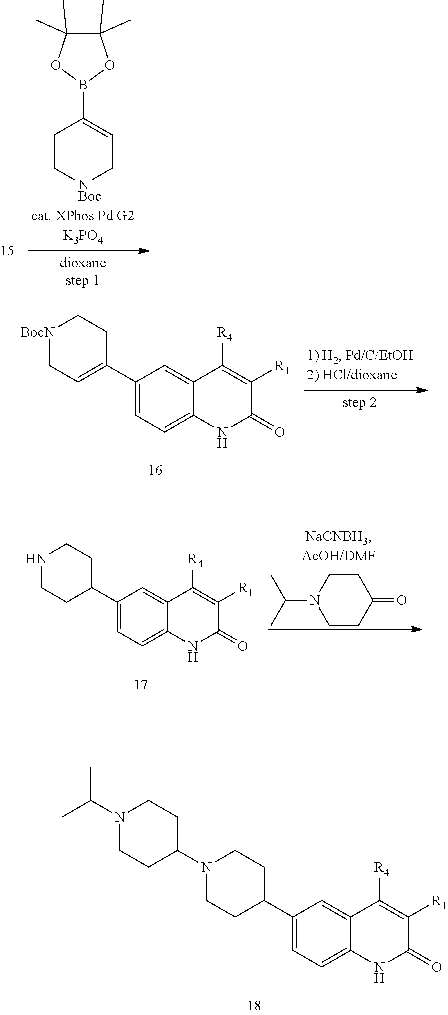

### Step 1. General Procedure for the Formation of Compound 16:

To a glass vial equipped with magnetic stirrer was added Compound 15 (1.0 equiv.), tert-butyl 4-(4,4,5,5-tetramethyl-1,3,2-dioxaborolan-2-yl)-3,6-dihydropyridine-1(2H)-carboxylate (1.5 equiv.), XPhos Pd G2 (0.1 equiv.), 3 M K₃PO₄ (3.0 equiv.) and dioxane (0.2 M). The mixture was heated to 90° C. under N₂ for 2 h. The reaction mixture was subjected to flash column chromatography and product-containing fractions were pooled and concentrated under reduced pressure.

### Example of Compound 16: Synthesis of tert-butyl 4-(4-ethyl-3-(4-(methylsulfonyl) phenyl)-2-oxo-1,2-dihydroquinolin-6-yl)-3,6-dihydropyridine-1(2H)-carboxylate

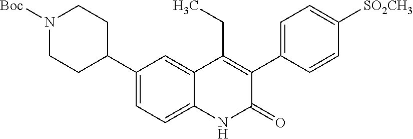 [SMILES: CCc1c(-c2ccc(S(C)(=O)=O)cc2)c(=O)[nH]c2ccc(C3CCN(C(=O)OC(C)(C)C)CC3)cc12]

To an 8 mL glass vial equipped with magnetic stirrer was added 6-bromo-4-ethyl-3-(4-(methylsulfonyl)phenyl)quinolin-2(1H)-one (135 mg, 0.33 mmol), tert-butyl 4-(4,4,5,5-tetramethyl-1,3,2-dioxaborolan-2-yl)-3,6-dihydropyridine-1(2H)-carboxylate (154 ng, 0.50 mmol), XPhos Pd G2 (26 mg, 0.033 nmol), 3 M K₃PO₄ (0.33 mL, 10 mmol) and dioxane (1.6 mL). The reaction mixture was heated to 90° C. under N₂ for 2 h. The reaction mixture was subjected to flash column chromatography and product-containing fractions were pooled and concentrated under reduced pressure to afford the title compound (108 mg, 0.21 nmol, 64% yield). Analytical Method 1: Observed mass: 509.1 (MH⁺); Retention Time: 0.958 min.

### Step 2. General Procedure for the Formation of Compound 17:

To a heavy-walled round bottomed flask equipped with magnetic stirrer was added Compound 16 (1.0 equiv.), wet Pd/C (0.1 equiv.), and ethanol (minimal volume to dissolve residue). The flask was evacuated and backfilled with N₂ three times. Them the flask was evacuated and backfilled with H₂ (50 psi) and stirred at 23° C. for 1 h. The reaction mixture was filtered through Celite and concentrated under reduced pressure. The residue was dissolved in 4 M HCl/dioxane (0.2 M) and stirred for 1 h at 23° C. and concentrated under reduced pressure to afford Compound 17 as a hydrochloride salt.

### Example of Compound 17: Synthesis of 4-ethyl-3-(4-(methylsulfonyl)phenyl)-6-(piperidin-4-yl)quinolin-2(1H)-one

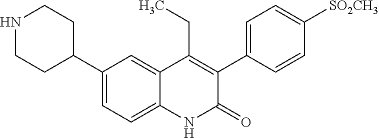 [SMILES: CCc1c(-c2ccc(S(C)(=O)=O)cc2)c(=O)[nH]c2ccc(C3CCNCC3)cc12]

To a 250 mL heavy-walled round-bottomed flask equipped with magnetic stirrer was added tert-butyl 4-(4-ethyl-3-(4-(methylsulfonyl)phenyl)-2-oxo-1,2-dihydroquinolin-6-yl)-3,6-dihydropyridine-1(2H)-carboxylate (108 mg, 0.21 mmol), wet Pd/C (71 ng, 0.033 mmol), and ethanol (10 mL). The flask was evacuated and backfilled with N₂ three times. Then, the flask was evacuate d and backfilled with H₂ (50 psi) and stirred at 23° C. for 1 h. The reaction mixture was filtered through Celite and concentrated under reduced pressure. The residue was dissolved in 4 M HCl/dioxane (1.5 mL), stirred for 1 h at 23° C., and concentrated under reduced pressure. The material was further purified by reverse phase preparative HPLC (Prep Method 2) to afford the title compound as the hydrochloride salt (1.6 mg, 0.0039 mmol, 1% yield). Analytical Method 5: Purity: 100%; Observed mass: 411.0 (MH⁺); Retention Time: 1.02 min; Analytical Method 6: Purity: 100%; Observed mass: 411.0 (MH⁺); Retention Time: 1.02 mii.

### Step 3. General Procedure for the Formation of Compound 18:

To a glass vial equipped with magnetic stirrer was added Compound 17 as the HCl salt (1.0 equiv.), 1-isopropylpiperidin-4-one (10 equiv), MgSO₄ (10 equiv.), and 10/1 DMF/AcOH (0.1 M). The reaction mixture was stirred for 15 min at 60° C. and then NaCNBH₃ (10 equiv.) was added. The reaction mixture was stirred for 72 h at 60° C. The reaction was quenched with 1 M NaOH (20 volumes) and the reaction mixture was extracted with DCM (20 volumes). The organic layer was concentrated under reduced pressure and the residue was dissolved in DMF and subjected to preparative reverse-phase HPLC. Product containing fractions were concentrated by centrifugal evaporation to afford Compound 18.

### Example of Compound 18: Synthesis of 4-ethyl-6-(1′-isopropyl-[1,4′-bipiperidin]-4-yl)-3-(4-(methylsulfonyl)phenyl)quinolin-2(1H)-one

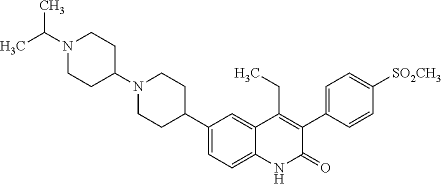 [SMILES: CCc1c(-c2ccc(S(C)(=O)=O)cc2)c(=O)[nH]c2ccc(C3CCN(C4CCN(C(C)C)CC4)CC3)cc12]

To a glass vial equipped with magnetic stirrer was added Compound 17 as the HCl salt (28 mg, 0.063 mmol), 1-isopropylpiperidin-4-one (88 mg, 0.63 mmol), MgSO₄ (75 mug, 0.63 mmol), and 10/1 DMF/AcOH (0.6 mL/0.06 mL). The reaction mixture was stirred for 15 min at 60° C. and then NaCNBH₃ (39 mg) was added. The reaction mixture was stirred for 72 h at 60° C. The reaction was quenched with 1 M NaOH (10 mL). The reaction mixture was extracted with DCM (10 mL). The organic laver was concentrated under reduced pressure and the residue was dissolved in DMF and subjected to preparative reverse-phase HPLC (Prep Method 1). Product-containing fractions were concentrated by centrifugal evaporation to afford the title compound (5.2 mg, 0.0097 mmol, 15% yield). Analytical Method 5: Purity: 100%; Observed mass: 536.3 (MH⁺); Retention Time: 0.94 min; Analytical Method 6: Purity: 100%; Observed mass: 536.5 (MH⁺); Retention Time: 1.12 min. ¹H NMR (500 MHz, DMSO-d₆) δ 11.84 (br s, 1H), 7.99 (d, J=8.0 Hz, 2H), 7.62 (s, 1H), 7.50 (d, J=7.9 Hz, 2H), 7.45 (br d, J=8.8 Hz, 1H), 7.30 (d, J=8.5 Hz, 1H), 3.28 (s, 3H), 3.04-2.95 (m, 2H), 2.89 (br dd, J=5.1, 1.9 Hz, 2H), 2.79-2.72 (m, 1H), 2.67-2.59 (m, 3H), 2.31-2.12 (m, 4H), 1.84-1.74 (m, 4H), 1.73-1.62 (m, 2H), 1.55-1.30 (m, 3H), 1.09 (br t, J=7.4 Hz, 3H), 0.98 (br d, J=6.5 Hz, 6H).

### General Scheme for the Synthesis of Compound 19:

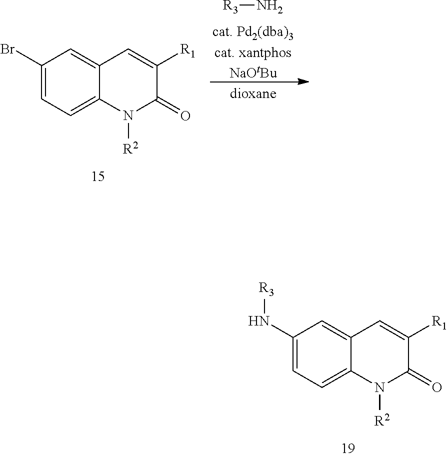

### General Procedure for the Formation of Compound 19:

To a glass vial equipped with magnetic stirrer was added Compound 15 (1.0 equiv.), amine (1.2 equiv.), Pd₂(dba)₃ (0.1 equiv.), Xantphos (0.1 equiv.), NaOtBu (2.0 equiv.), and dioxane (0.2 M). The reaction mixture was sparged with N₂ and vent needle for 5 min and stirred at 90° C. under N₂ for 1 h. The mixture was subjected to normal phase flash column chromatography followed by preparative reverse-phase HPLC. Product-containing fractions were concentrated by centrifugal evaporation to afford Compound 19.

### Example of Compound 19: Synthesis of 6-((4-(4-isopropylpiperazin-1-yl)phenyl)amino)-1-methyl-3-(4-(methylsulfonyl)phenyl)quinolin-2(1H)-one (Example 3)

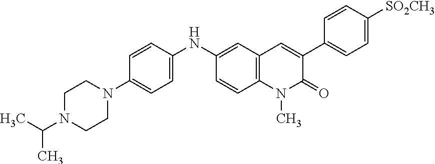 [SMILES: CC(C)N1CCN(c2ccc(Nc3ccc4c(c3)cc(-c3ccc(S(C)(=O)=O)cc3)c(=O)n4C)cc2)CC1]

To an 8 mL glass vial equipped with magnetic stirrer was added 6-bromo-1-methyl-3-(4-(methylsulfonyl)phenyl)quinolin-2(1H)-one (50 mg, 0.13 mmol), 4-(4-isopropylpiperazin-1-yl)aniline (33.5 mg, 0.15 mmol), Pd₂(dba)₃ (11.7 mg, 0.013 mmol), Xantphos (7.4 rug, 0.013 mmol), NaOtBu (24.5 mg, 0.26 mmol), and dioxane (0.6 mL). The reaction mixture was sparged with N₂ and vent needle for 5 min and stirred at 90° C. under N₂ for 1 h. The mixture was subjected to normal phase flash column chromatography (DCM/MeOH) followed by preparative reverse-phase HPLC (Prep Method 1). Product-containing fractions were concentrated by centrifugal evaporation to afford the title compound (5.8 mg, 0.011 mmol, 9% yield). Analytical Method 5: Purity: 100%; Observed mass: 531.1 (MH⁺); Retention Time: 1.30 min; Analytical Method 6: Purity: 100%; Observed mass: 531.1 (MH⁺); Retention Time: 1.47 min. ¹H NMR (500 MHz, DMSO-d₆) δ 8.12 (s, 1H), 8.02-7.98 (m, 2H), 7.97-7.93 (m, 2H), 7.91 (s, 1H), 7.45 (d, J=9.1 Hz, 1H), 7.32 (d, J=2.4 Hz, 1H, 7.28 (dd, J=9.0, 2.5 Hz, 1H), 7.03 (d, J=8.9 Hz, 2H), 6.89 (d, J=8.9 Hz, 2H), 3.67 (s, 3H), 3.24 (s, 3H), 3.06-3.00 (m, 4H), 2.69-2.62 (m, 1H), 2.59-2.55 (m, 41H), 1.00 (d, J=6.5 Hz, 6H).

The following Examples were prepared according to the general methods described herein above using appropriate starting materials, reagents and conditions.

| Ex. No. | Structure | Method |
|---|---|---|
| 4 | 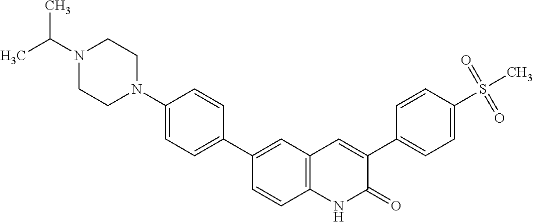 [SMILES: CC(C)N1CCN(c2ccc(-c3ccc4[nH]c(=O)c(-c5ccc(S(C)(=O)=O)cc5)cc4c3)cc2)CC1] | Ex. 2 |
| 5 | 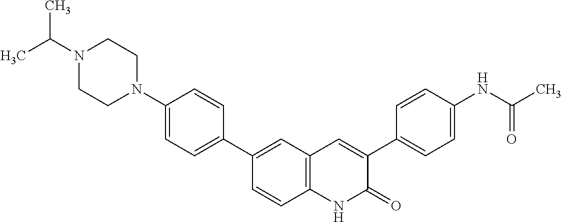 [SMILES: CC(=O)Nc1ccc(-c2cc3cc(-c4ccc(N5CCN(C(C)C)CC5)cc4)ccc3[nH]c2=O)cc1] | Ex. 2 |
| 6 | 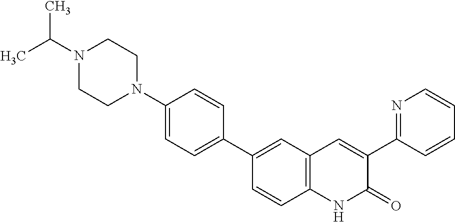 [SMILES: CC(C)N1CCN(c2ccc(-c3ccc4[nH]c(=O)c(-c5ccccn5)cc4c3)cc2)CC1] | Ex. 2 |
| 7 | 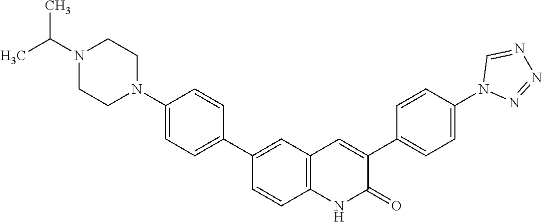 [SMILES: CC(C)N1CCN(c2ccc(-c3ccc4[nH]c(=O)c(-c5ccc(-n6cnnn6)cc5)cc4c3)cc2)CC1] | Ex. 2 |
| 8 | 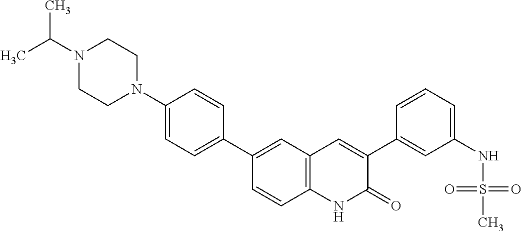 [SMILES: CC(C)N1CCN(c2ccc(-c3ccc4[nH]c(=O)c(-c5cccc(NS(C)(=O)=O)c5)cc4c3)cc2)CC1] | Ex. 2 |
| 9 | 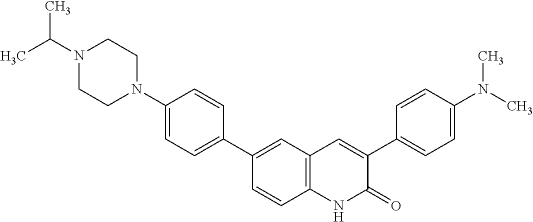 [SMILES: CC(C)N1CCN(c2ccc(-c3ccc4[nH]c(=O)c(-c5ccc(N(C)C)cc5)cc4c3)cc2)CC1] | Ex. 2 |
| 10 | 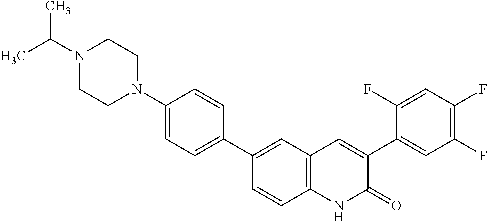 [SMILES: CC(C)N1CCN(c2ccc(-c3ccc4[nH]c(=O)c(-c5cc(F)c(F)cc5F)cc4c3)cc2)CC1] | Ex. 2 |
| 11 | 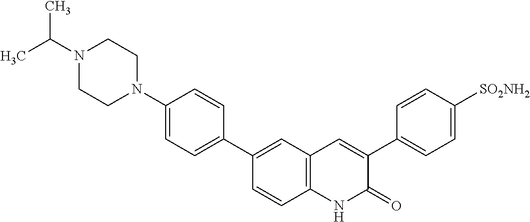 [SMILES: CC(C)N1CCN(c2ccc(-c3ccc4[nH]c(=O)c(-c5ccc(S(N)(=O)=O)cc5)cc4c3)cc2)CC1] | Ex. 2 |
| 12 | 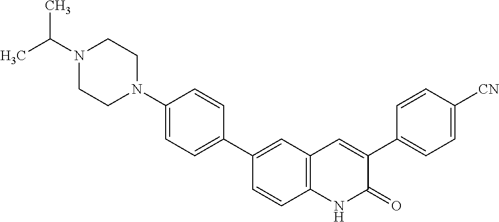 [SMILES: CC(C)N1CCN(c2ccc(-c3ccc4[nH]c(=O)c(-c5ccc(C#N)cc5)cc4c3)cc2)CC1] | Ex. 2 |
| 13 | 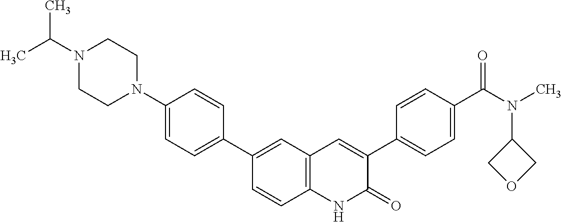 [SMILES: CC(C)N1CCN(c2ccc(-c3ccc4[nH]c(=O)c(-c5ccc(C(=O)N(C)C6COC6)cc5)cc4c3)cc2)CC1] | Ex. 9 |
| 14 | 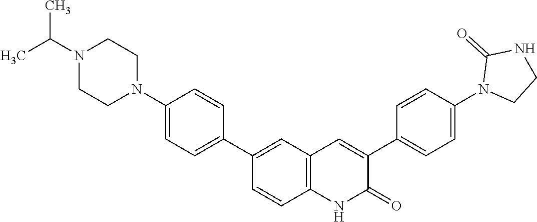 [SMILES: CC(C)N1CCN(c2ccc(-c3ccc4[nH]c(=O)c(-c5ccc(N6CCNC6=O)cc5)cc4c3)cc2)CC1] | Ex. 11 |
| 15 | 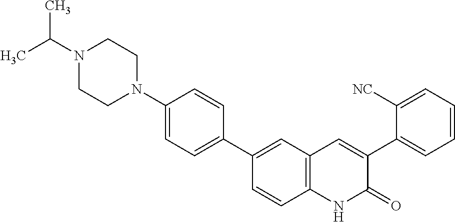 [SMILES: CC(C)N1CCN(c2ccc(-c3ccc4[nH]c(=O)c(-c5ccccc5C#N)cc4c3)cc2)CC1] | Ex. 2 |
| 16 | 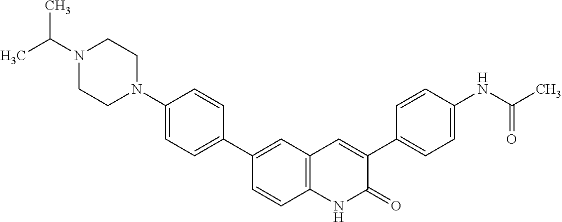 [SMILES: CC(=O)Nc1ccc(-c2cc3cc(-c4ccc(N5CCN(C(C)C)CC5)cc4)ccc3[nH]c2=O)cc1] | Ex. 2 |
| 17 | 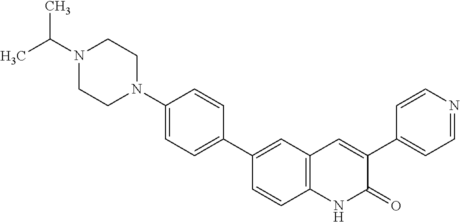 [SMILES: CC(C)N1CCN(c2ccc(-c3ccc4[nH]c(=O)c(-c5ccncc5)cc4c3)cc2)CC1] | Ex. 2 |
| 18 | 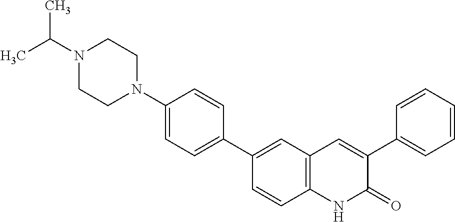 [SMILES: CC(C)N1CCN(c2ccc(-c3ccc4[nH]c(=O)c(-c5ccccc5)cc4c3)cc2)CC1] | Ex. 2 |
| 19 | 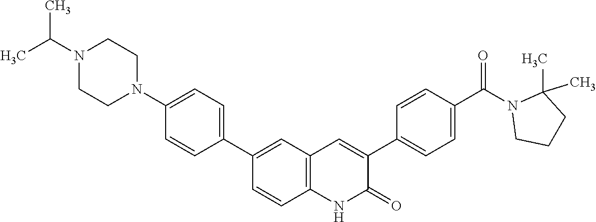 [SMILES: CC(C)N1CCN(c2ccc(-c3ccc4[nH]c(=O)c(-c5ccc(C(=O)N6CCCC6(C)C)cc5)cc4c3)cc2)CC1] | Ex. 9 |
| 20 | 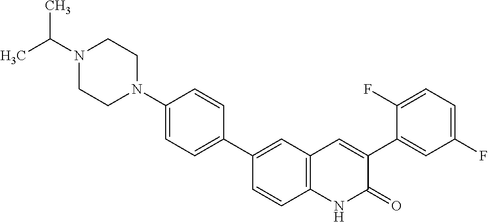 [SMILES: CC(C)N1CCN(c2ccc(-c3ccc4[nH]c(=O)c(-c5cc(F)ccc5F)cc4c3)cc2)CC1] | Ex. 2 |
| 21 | 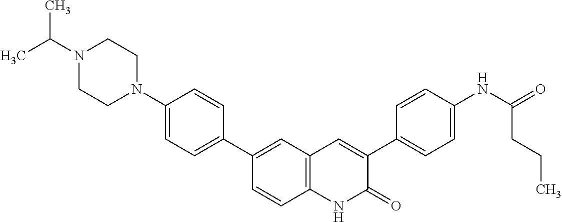 [SMILES: CCCC(=O)Nc1ccc(-c2cc3cc(-c4ccc(N5CCN(C(C)C)CC5)cc4)ccc3[nH]c2=O)cc1] | Ex. 7 |
| 22 | 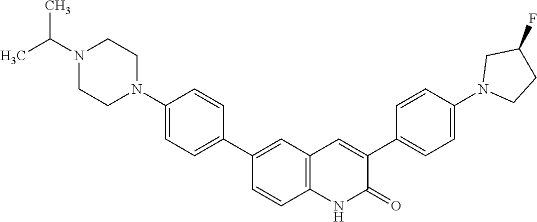 [SMILES: CC(C)N1CCN(c2ccc(-c3ccc4[nH]c(=O)c(-c5ccc(N6CC[C@H](F)C6)cc5)cc4c3)cc2)CC1] | Ex. 11 |
| 23 | 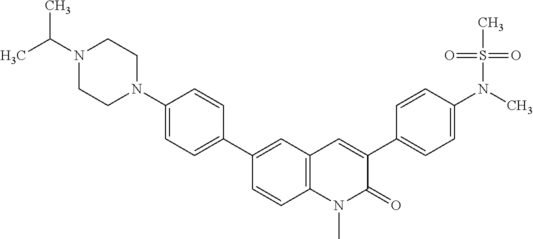 [SMILES: CC(C)N1CCN(c2ccc(-c3ccc4c(c3)cc(-c3ccc(N(C)S(C)(=O)=O)cc3)c(=O)n4C)cc2)CC1] | Ex. 5 |
| 24 | 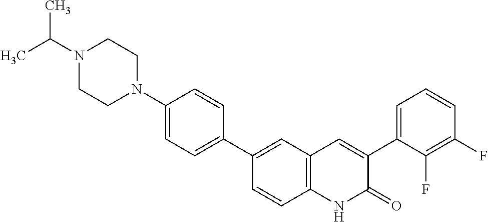 [SMILES: CC(C)N1CCN(c2ccc(-c3ccc4[nH]c(=O)c(-c5cccc(F)c5F)cc4c3)cc2)CC1] | Ex. 2 |
| 25 | 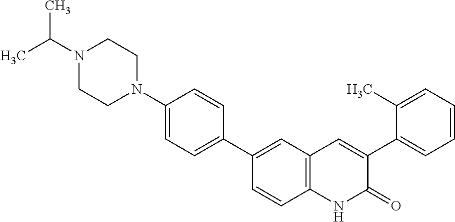 [SMILES: Cc1ccccc1-c1cc2cc(-c3ccc(N4CCN(C(C)C)CC4)cc3)ccc2[nH]c1=O] | Ex. 2 |
| 26 | 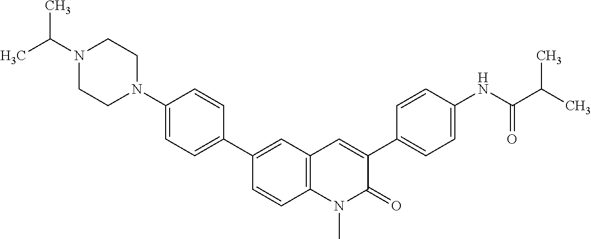 [SMILES: CC(C)C(=O)Nc1ccc(-c2cc3cc(-c4ccc(N5CCN(C(C)C)CC5)cc4)ccc3n(C)c2=O)cc1] | Ex. 7 |
| 27 | 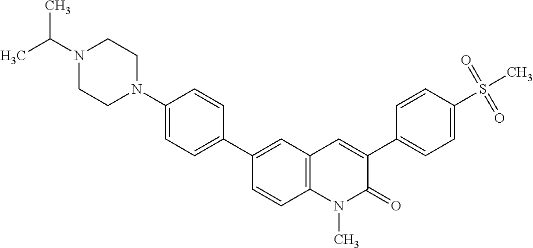 [SMILES: CC(C)N1CCN(c2ccc(-c3ccc4c(c3)cc(-c3ccc(S(C)(=O)=O)cc3)c(=O)n4C)cc2)CC1] | Ex. 5 |
| 28 | 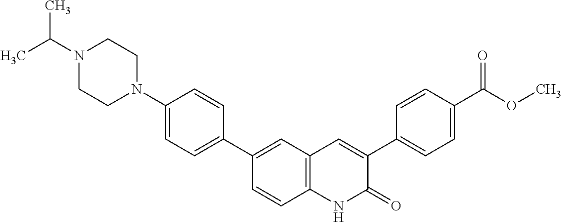 [SMILES: COC(=O)c1ccc(-c2cc3cc(-c4ccc(N5CCN(C(C)C)CC5)cc4)ccc3[nH]c2=O)cc1] | Ex. 2 |
| 29 | 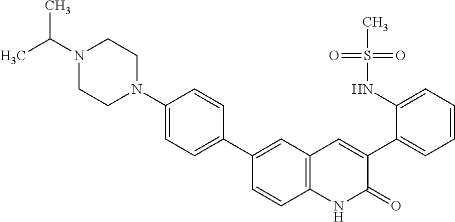 [SMILES: CC(C)N1CCN(c2ccc(-c3ccc4[nH]c(=O)c(-c5ccccc5NS(C)(=O)=O)cc4c3)cc2)CC1] | Ex. 2 |
| 30 | 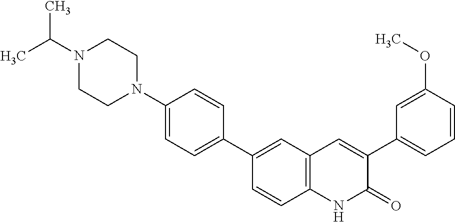 [SMILES: COc1cccc(-c2cc3cc(-c4ccc(N5CCN(C(C)C)CC5)cc4)ccc3[nH]c2=O)c1] | Ex. 2 |
| 31 | 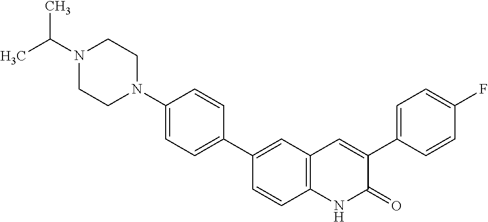 [SMILES: CC(C)N1CCN(c2ccc(-c3ccc4[nH]c(=O)c(-c5ccc(F)cc5)cc4c3)cc2)CC1] | Ex. 2 |
| 32 | 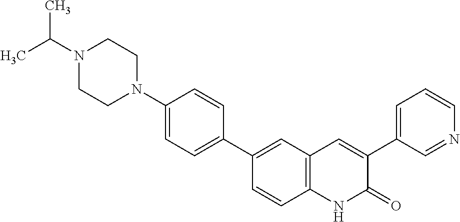 [SMILES: CC(C)N1CCN(c2ccc(-c3ccc4[nH]c(=O)c(-c5cccnc5)cc4c3)cc2)CC1] | Ex. 2 |
| 33 | 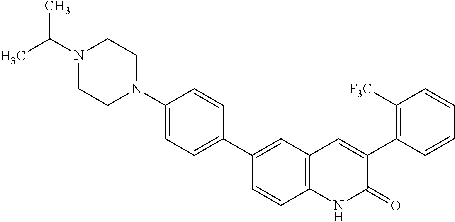 [SMILES: CC(C)N1CCN(c2ccc(-c3ccc4[nH]c(=O)c(-c5ccccc5C(F)(F)F)cc4c3)cc2)CC1] | Ex. 2 |
| 34 | 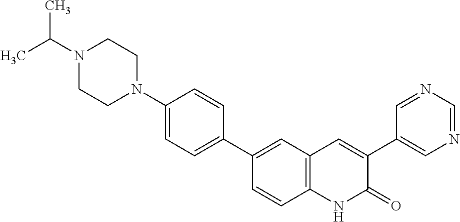 [SMILES: CC(C)N1CCN(c2ccc(-c3ccc4[nH]c(=O)c(-c5cncnc5)cc4c3)cc2)CC1] | Ex. 2 |
| 35 | 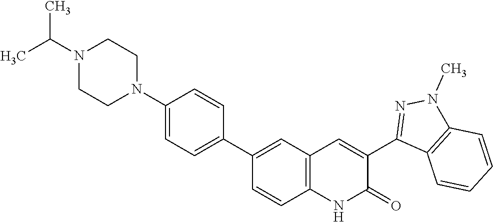 [SMILES: CC(C)N1CCN(c2ccc(-c3ccc4[nH]c(=O)c(-c5nn(C)c6ccccc56)cc4c3)cc2)CC1] | Ex. 2 |
| 36 | 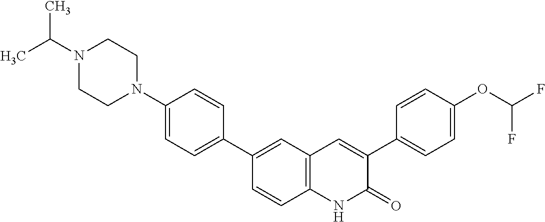 [SMILES: CC(C)N1CCN(c2ccc(-c3ccc4[nH]c(=O)c(-c5ccc(OC(F)F)cc5)cc4c3)cc2)CC1] | Ex. 2 |
| 37 | 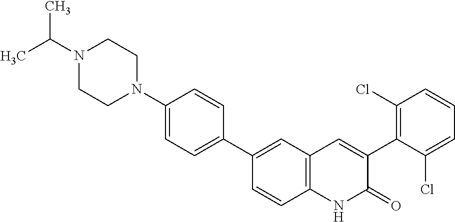 [SMILES: CC(C)N1CCN(c2ccc(-c3ccc4[nH]c(=O)c(-c5c(Cl)cccc5Cl)cc4c3)cc2)CC1] | Ex. 2 |
| 38 | 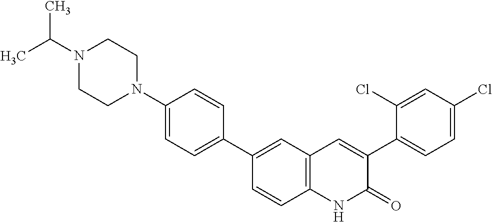 [SMILES: CC(C)N1CCN(c2ccc(-c3ccc4[nH]c(=O)c(-c5ccc(Cl)cc5Cl)cc4c3)cc2)CC1] | Ex. 2 |
| 39 | 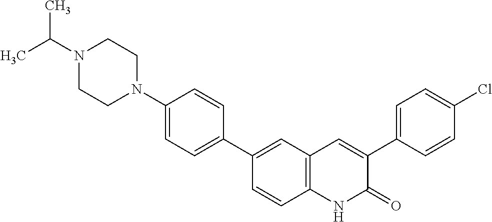 [SMILES: CC(C)N1CCN(c2ccc(-c3ccc4[nH]c(=O)c(-c5ccc(Cl)cc5)cc4c3)cc2)CC1] | Ex. 2 |
| 40 | 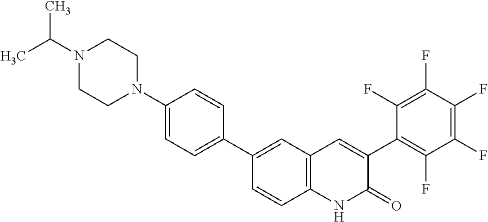 [SMILES: CC(C)N1CCN(c2ccc(-c3ccc4[nH]c(=O)c(-c5c(F)c(F)c(F)c(F)c5F)cc4c3)cc2)CC1] | Ex. 2 |
| 41 | 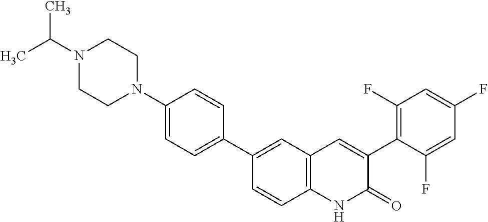 [SMILES: CC(C)N1CCN(c2ccc(-c3ccc4[nH]c(=O)c(-c5c(F)cc(F)cc5F)cc4c3)cc2)CC1] | Ex. 2 |
| 42 | 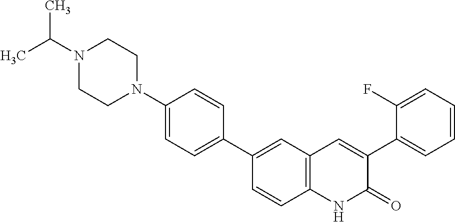 [SMILES: CC(C)N1CCN(c2ccc(-c3ccc4[nH]c(=O)c(-c5ccccc5F)cc4c3)cc2)CC1] | Ex. 2 |
| 43 | 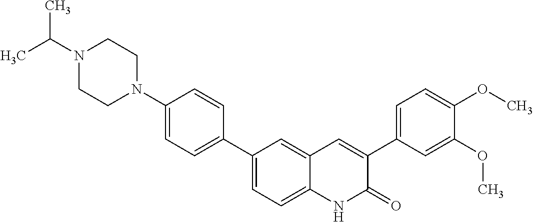 [SMILES: COc1ccc(-c2cc3cc(-c4ccc(N5CCN(C(C)C)CC5)cc4)ccc3[nH]c2=O)cc1OC] | Ex. 2 |
| 44 | 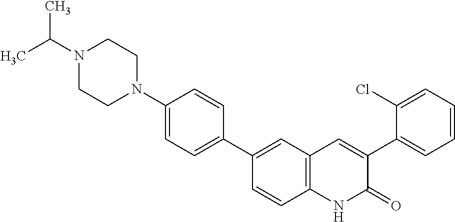 [SMILES: CC(C)N1CCN(c2ccc(-c3ccc4[nH]c(=O)c(-c5ccccc5Cl)cc4c3)cc2)CC1] | Ex. 2 |
| 45 | 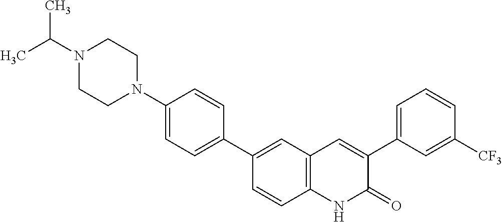 [SMILES: CC(C)N1CCN(c2ccc(-c3ccc4[nH]c(=O)c(-c5cccc(C(F)(F)F)c5)cc4c3)cc2)CC1] | Ex. 2 |
| 46 | 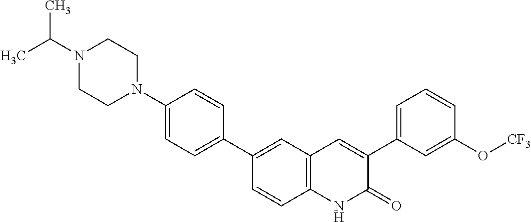 [SMILES: CC(C)N1CCN(c2ccc(-c3ccc4[nH]c(=O)c(-c5cccc(OC(F)(F)F)c5)cc4c3)cc2)CC1] | Ex. 2 |
| 47 | 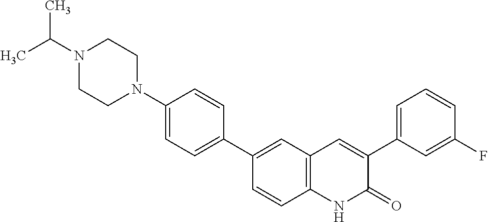 [SMILES: CC(C)N1CCN(c2ccc(-c3ccc4[nH]c(=O)c(-c5cccc(F)c5)cc4c3)cc2)CC1] | Ex. 2 |
| 48 | 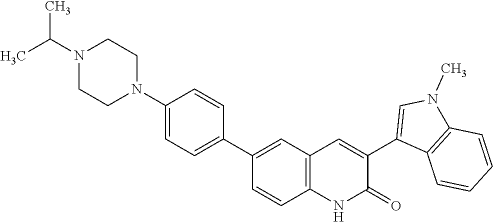 [SMILES: CC(C)N1CCN(c2ccc(-c3ccc4[nH]c(=O)c(-c5cn(C)c6ccccc56)cc4c3)cc2)CC1] | Ex. 2 |
| 49 | 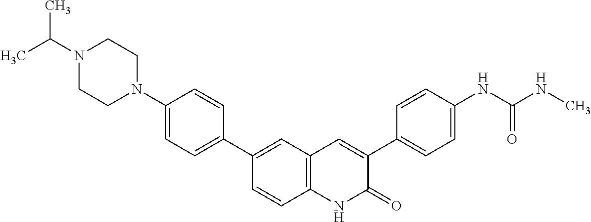 [SMILES: CNC(=O)Nc1ccc(-c2cc3cc(-c4ccc(N5CCN(C(C)C)CC5)cc4)ccc3[nH]c2=O)cc1] | Ex. 11 |
| 50 | 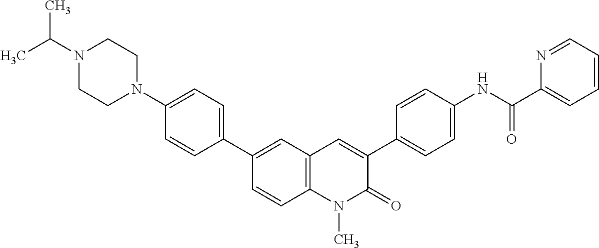 [SMILES: CC(C)N1CCN(c2ccc(-c3ccc4c(c3)cc(-c3ccc(NC(=O)c5ccccn5)cc3)c(=O)n4C)cc2)CC1] | Ex. 7 |
| 51 | 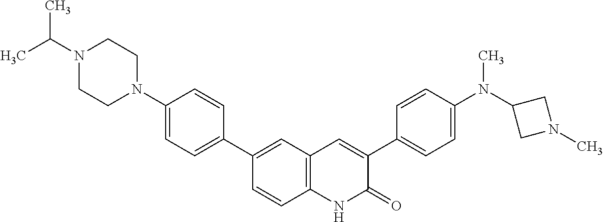 [SMILES: CC(C)N1CCN(c2ccc(-c3ccc4[nH]c(=O)c(-c5ccc(N(C)C6CN(C)C6)cc5)cc4c3)cc2)CC1] | Ex. 11 |
| 52 | 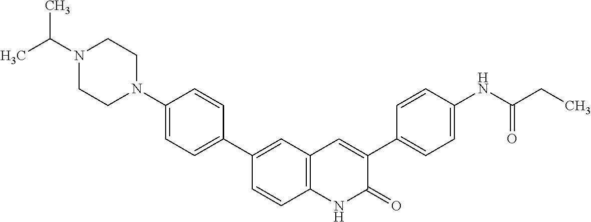 [SMILES: CCC(=O)Nc1ccc(-c2cc3cc(-c4ccc(N5CCN(C(C)C)CC5)cc4)ccc3[nH]c2=O)cc1] | Ex. 7 |
| 53 | 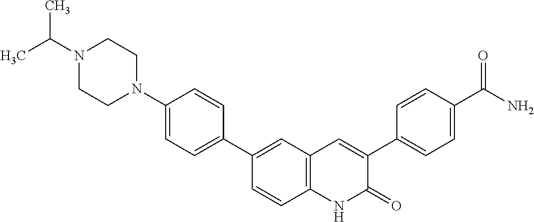 [SMILES: CC(C)N1CCN(c2ccc(-c3ccc4[nH]c(=O)c(-c5ccc(C(N)=O)cc5)cc4c3)cc2)CC1] | Ex. 9 |
| 54 | 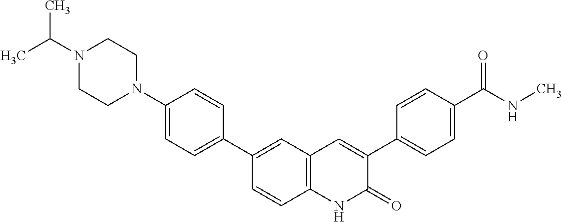 [SMILES: CNC(=O)c1ccc(-c2cc3cc(-c4ccc(N5CCN(C(C)C)CC5)cc4)ccc3[nH]c2=O)cc1] | Ex. 9 |
| 55 | 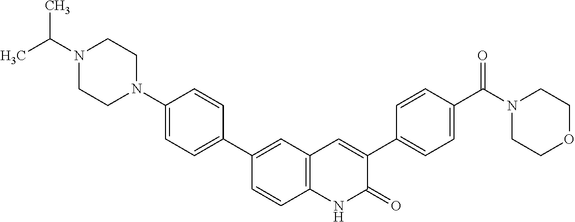 [SMILES: CC(C)N1CCN(c2ccc(-c3ccc4[nH]c(=O)c(-c5ccc(C(=O)N6CCOCC6)cc5)cc4c3)cc2)CC1] | Ex. 9 |
| 56 | 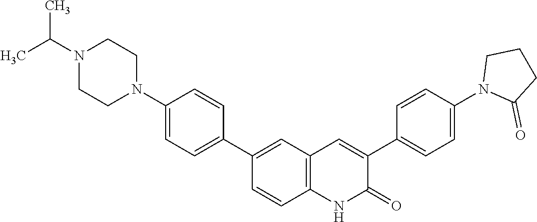 [SMILES: CC(C)N1CCN(c2ccc(-c3ccc4[nH]c(=O)c(-c5ccc(N6CCCC6=O)cc5)cc4c3)cc2)CC1] | Ex. 11 |
| 57 | 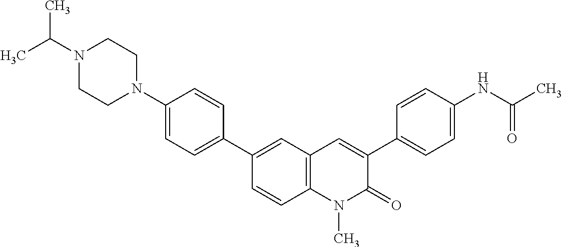 [SMILES: CC(=O)Nc1ccc(-c2cc3cc(-c4ccc(N5CCN(C(C)C)CC5)cc4)ccc3n(C)c2=O)cc1] | Ex. 7 |
| 58 | 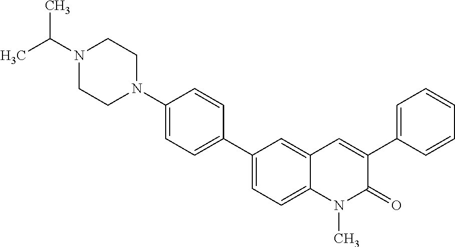 [SMILES: CC(C)N1CCN(c2ccc(-c3ccc4c(c3)cc(-c3ccccc3)c(=O)n4C)cc2)CC1] | Ex. 5 |
| 59 | 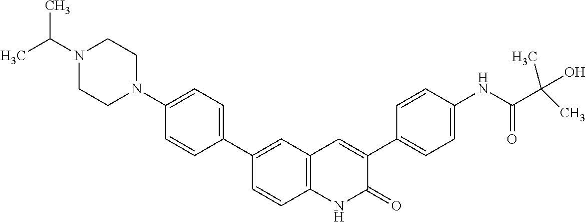 [SMILES: CC(C)N1CCN(c2ccc(-c3ccc4[nH]c(=O)c(-c5ccc(NC(=O)C(C)(C)O)cc5)cc4c3)cc2)CC1] | Ex. 7 |
| 60 | 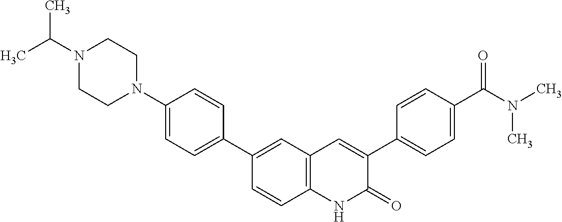 [SMILES: CC(C)N1CCN(c2ccc(-c3ccc4[nH]c(=O)c(-c5ccc(C(=O)N(C)C)cc5)cc4c3)cc2)CC1] | Ex. 9 |
| 61 | 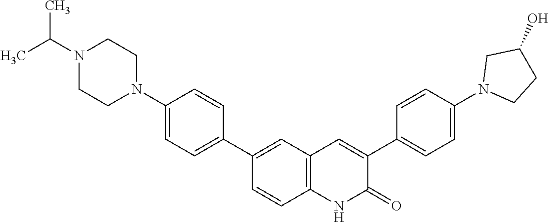 [SMILES: CC(C)N1CCN(c2ccc(-c3ccc4[nH]c(=O)c(-c5ccc(N6CC[C@@H](O)C6)cc5)cc4c3)cc2)CC1] | Ex. 11 |
| 62 | 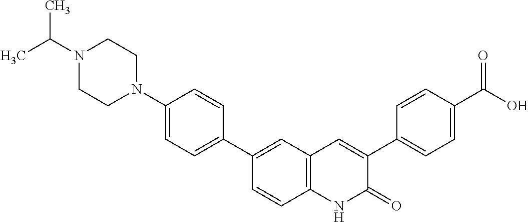 [SMILES: CC(C)N1CCN(c2ccc(-c3ccc4[nH]c(=O)c(-c5ccc(C(=O)O)cc5)cc4c3)cc2)CC1] | Ex. 8 |
| 63 | 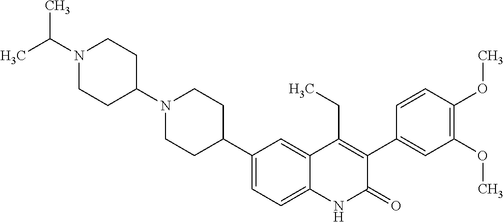 [SMILES: CCc1c(-c2ccc(OC)c(OC)c2)c(=O)[nH]c2ccc(C3CCN(C4CCN(C(C)C)CC4)CC3)cc12] | Ex. 18 |
| 64 | 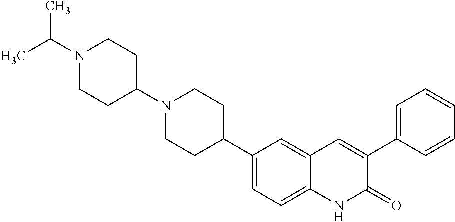 [SMILES: CC(C)N1CCC(N2CCC(c3ccc4[nH]c(=O)c(-c5ccccc5)cc4c3)CC2)CC1] | Ex. 18 |
| 65 | 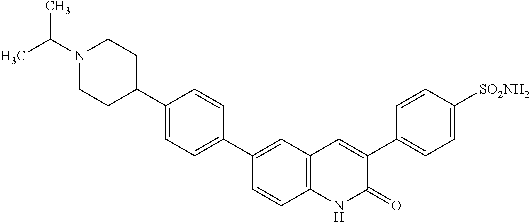 [SMILES: CC(C)N1CCC(c2ccc(-c3ccc4[nH]c(=O)c(-c5ccc(S(N)(=O)=O)cc5)cc4c3)cc2)CC1] | Ex. 2 |
| 66 | 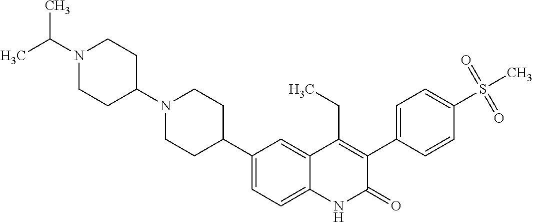 [SMILES: CCc1c(-c2ccc(S(C)(=O)=O)cc2)c(=O)[nH]c2ccc(C3CCN(C4CCN(C(C)C)CC4)CC3)cc12] | Ex. 18 |
| 67 | 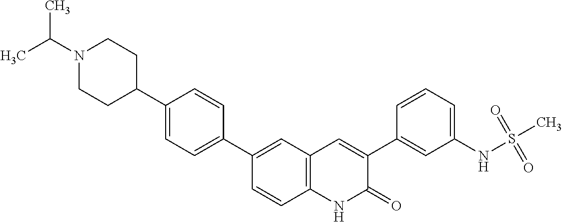 [SMILES: CC(C)N1CCC(c2ccc(-c3ccc4[nH]c(=O)c(-c5cccc(NS(C)(=O)=O)c5)cc4c3)cc2)CC1] | Ex. 2 |
| 68 | 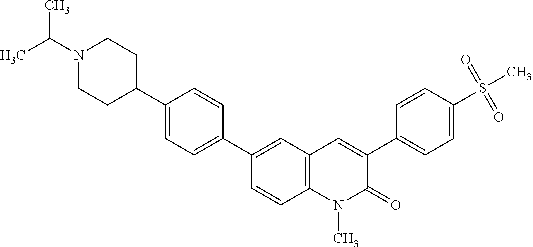 [SMILES: CC(C)N1CCC(c2ccc(-c3ccc4c(c3)cc(-c3ccc(S(C)(=O)=O)cc3)c(=O)n4C)cc2)CC1] | Ex. 5 |
| 69 | 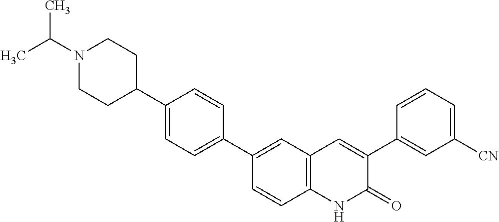 [SMILES: CC(C)N1CCC(c2ccc(-c3ccc4[nH]c(=O)c(-c5cccc(C#N)c5)cc4c3)cc2)CC1] | Ex. 2 |
| 70 | 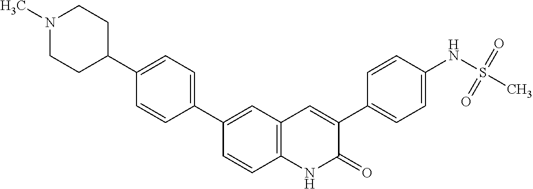 | Ex. 2 |
| 71 | 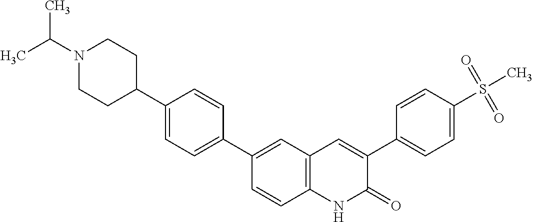 [SMILES: CC(C)N1CCC(c2ccc(-c3ccc4[nH]c(=O)c(-c5ccc(S(C)(=O)=O)cc5)cc4c3)cc2)CC1] | Ex. 2 |
| 72 | 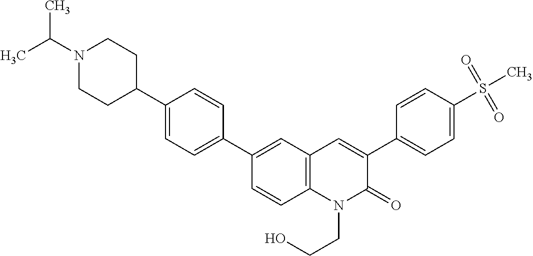 [SMILES: CC(C)N1CCC(c2ccc(-c3ccc4c(c3)cc(-c3ccc(S(C)(=O)=O)cc3)c(=O)n4CCO)cc2)CC1] | Ex. 5 |
| 73 | 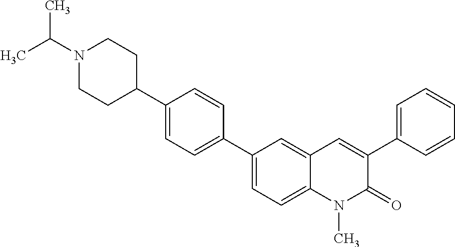 [SMILES: CC(C)N1CCC(c2ccc(-c3ccc4c(c3)cc(-c3ccccc3)c(=O)n4C)cc2)CC1] | Ex. 5 |
| 74 | 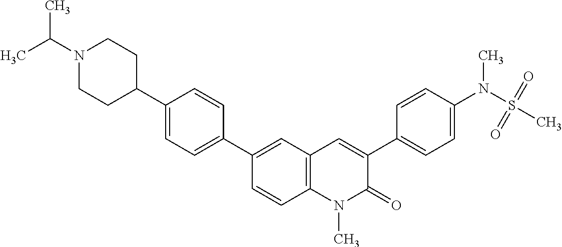 [SMILES: CC(C)N1CCC(c2ccc(-c3ccc4c(c3)cc(-c3ccc(N(C)S(C)(=O)=O)cc3)c(=O)n4C)cc2)CC1] | Ex. 5 |
| 75 | 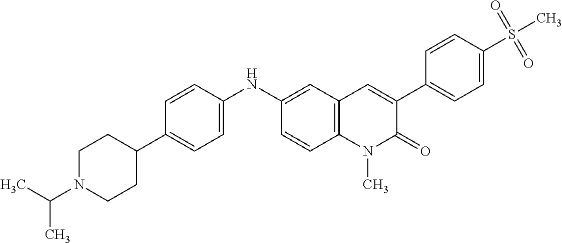 [SMILES: CC(C)N1CCC(c2ccc(Nc3ccc4c(c3)cc(-c3ccc(S(C)(=O)=O)cc3)c(=O)n4C)cc2)CC1] | Ex. 19 |
| 76 | 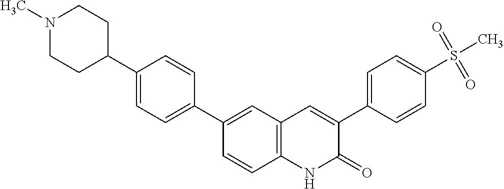 | Ex. 2 |
| 77 | 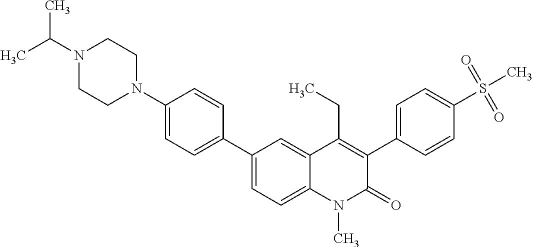 [SMILES: CCc1c(-c2ccc(S(C)(=O)=O)cc2)c(=O)n(C)c2ccc(-c3ccc(N4CCN(C(C)C)CC4)cc3)cc12] | Ex. 18 |
| 78 | 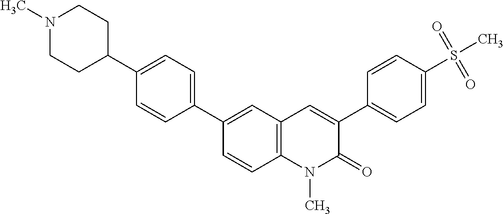 | Ex. 5 |
| 79 | 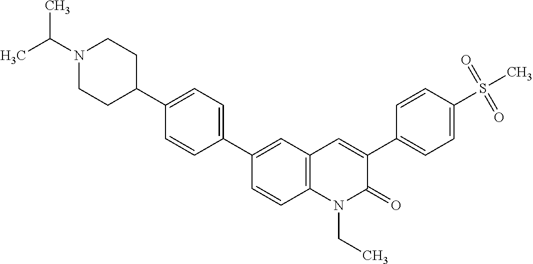 [SMILES: CCn1c(=O)c(-c2ccc(S(C)(=O)=O)cc2)cc2cc(-c3ccc(C4CCN(C(C)C)CC4)cc3)ccc21] | Ex. 5 |
| 80 | 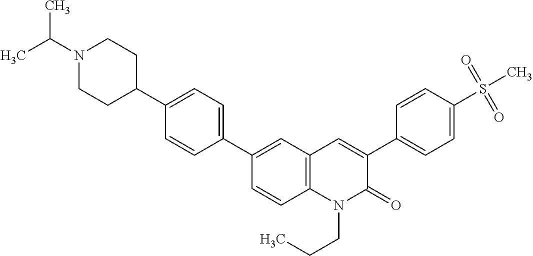 [SMILES: CCCn1c(=O)c(-c2ccc(S(C)(=O)=O)cc2)cc2cc(-c3ccc(C4CCN(C(C)C)CC4)cc3)ccc21] | Ex. 5 |
| 81 | 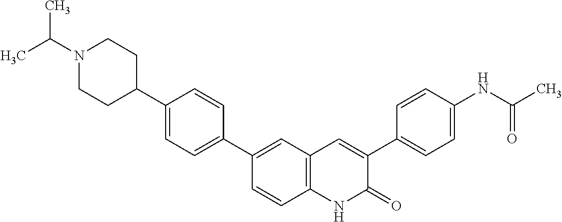 [SMILES: CC(=O)Nc1ccc(-c2cc3cc(-c4ccc(C5CCN(C(C)C)CC5)cc4)ccc3[nH]c2=O)cc1] | Ex. 7 |
| 82 | 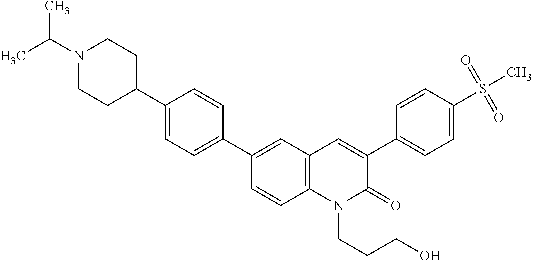 [SMILES: CC(C)N1CCC(c2ccc(-c3ccc4c(c3)cc(-c3ccc(S(C)(=O)=O)cc3)c(=O)n4CCCO)cc2)CC1] | Ex. 5 |
| 83 | 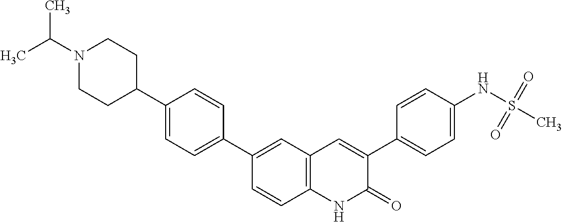 [SMILES: CC(C)N1CCC(c2ccc(-c3ccc4[nH]c(=O)c(-c5ccc(NS(C)(=O)=O)cc5)cc4c3)cc2)CC1] | Ex. 5 |
| 84 | 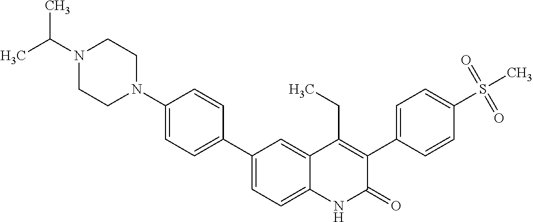 [SMILES: CCc1c(-c2ccc(S(C)(=O)=O)cc2)c(=O)[nH]c2ccc(-c3ccc(N4CCN(C(C)C)CC4)cc3)cc12] | Ex. 18 |
| 85 | 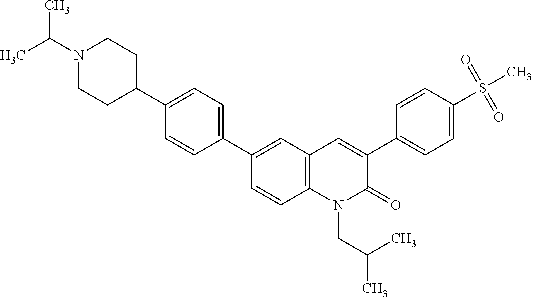 [SMILES: CC(C)Cn1c(=O)c(-c2ccc(S(C)(=O)=O)cc2)cc2cc(-c3ccc(C4CCN(C(C)C)CC4)cc3)ccc21] | Ex. 5 |
| 86 | 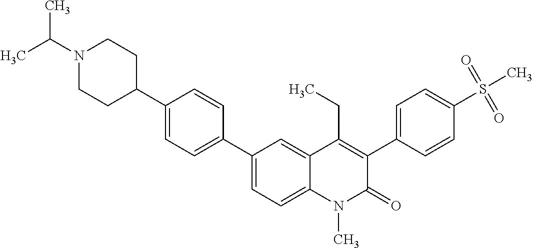 [SMILES: CCc1c(-c2ccc(S(C)(=O)=O)cc2)c(=O)n(C)c2ccc(-c3ccc(C4CCN(C(C)C)CC4)cc3)cc12] | Ex. 18 |
| 87 | 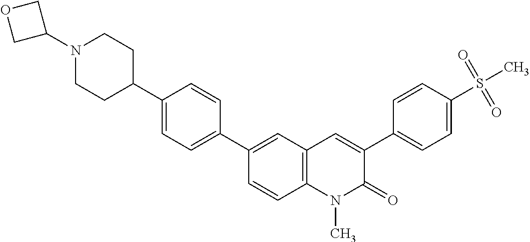 [SMILES: Cn1c(=O)c(-c2ccc(S(C)(=O)=O)cc2)cc2cc(-c3ccc(C4CCN(C5COC5)CC4)cc3)ccc21] | Ex. 5 |
| 88 | 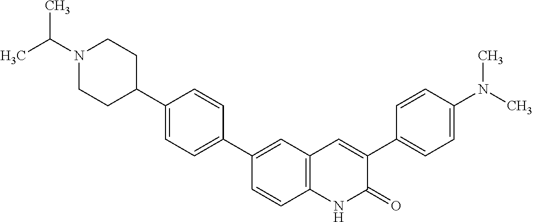 [SMILES: CC(C)N1CCC(c2ccc(-c3ccc4[nH]c(=O)c(-c5ccc(N(C)C)cc5)cc4c3)cc2)CC1] | Ex. 2 |
| 89 | 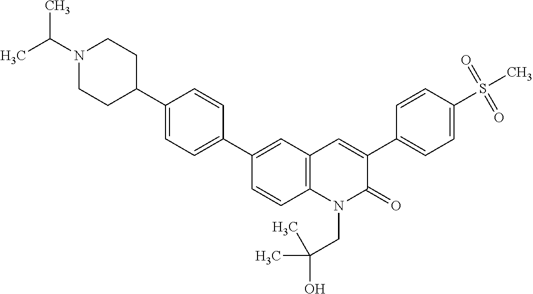 [SMILES: CC(C)N1CCC(c2ccc(-c3ccc4c(c3)cc(-c3ccc(S(C)(=O)=O)cc3)c(=O)n4CC(C)(C)O)cc2)CC1] | Ex. 5 |
| 90 | 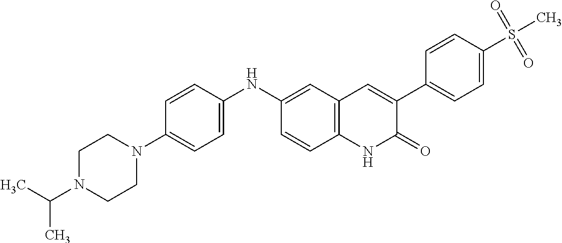 [SMILES: CC(C)N1CCN(c2ccc(Nc3ccc4[nH]c(=O)c(-c5ccc(S(C)(=O)=O)cc5)cc4c3)cc2)CC1] | Ex. 19 |
| 91 | 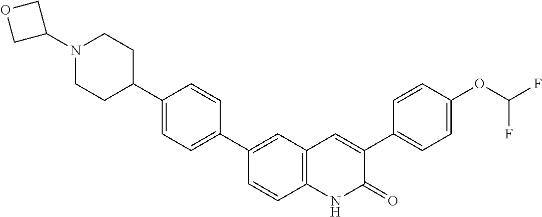 [SMILES: O=c1[nH]c2ccc(-c3ccc(C4CCN(C5COC5)CC4)cc3)cc2cc1-c1ccc(OC(F)F)cc1] | Ex. 2 |
| 92 | 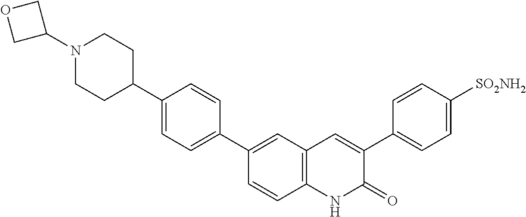 [SMILES: NS(=O)(=O)c1ccc(-c2cc3cc(-c4ccc(C5CCN(C6COC6)CC5)cc4)ccc3[nH]c2=O)cc1] | Ex. 2 |
| 93 | 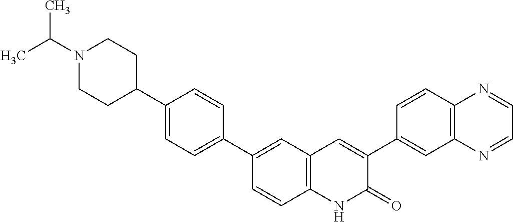 [SMILES: CC(C)N1CCC(c2ccc(-c3ccc4[nH]c(=O)c(-c5ccc6nccnc6c5)cc4c3)cc2)CC1] | Ex. 2 |
| 94 | 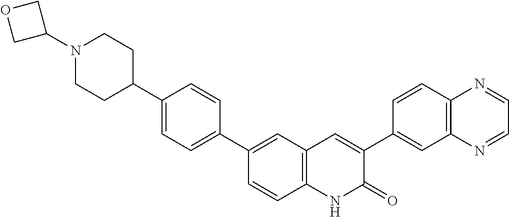 [SMILES: O=c1[nH]c2ccc(-c3ccc(C4CCN(C5COC5)CC4)cc3)cc2cc1-c1ccc2nccnc2c1] | Ex. 2 |
| 95 | 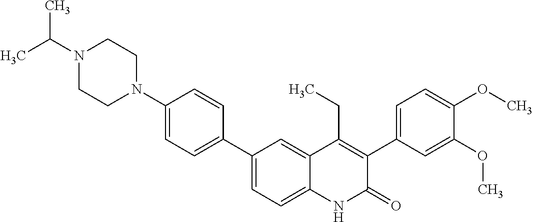 [SMILES: CCc1c(-c2ccc(OC)c(OC)c2)c(=O)[nH]c2ccc(-c3ccc(N4CCN(C(C)C)CC4)cc3)cc12] | Ex. 18 |
| 96 | 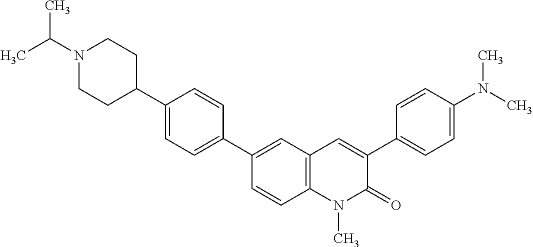 [SMILES: CC(C)N1CCC(c2ccc(-c3ccc4c(c3)cc(-c3ccc(N(C)C)cc3)c(=O)n4C)cc2)CC1] | Ex. 2 |
| 97 | 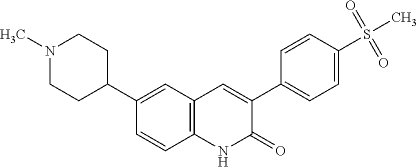 | Ex. 2 |
| 98 | 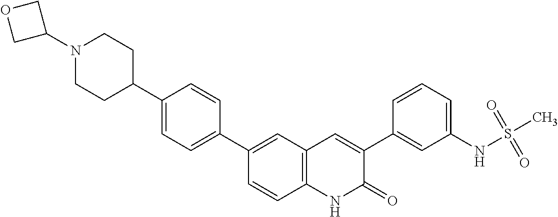 [SMILES: CS(=O)(=O)Nc1cccc(-c2cc3cc(-c4ccc(C5CCN(C6COC6)CC5)cc4)ccc3[nH]c2=O)c1] | Ex. 2 |
| 99 | 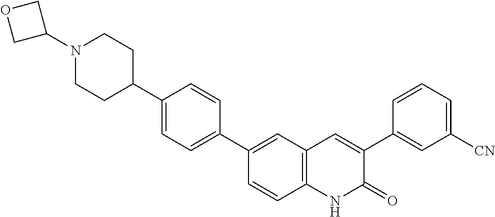 [SMILES: N#Cc1cccc(-c2cc3cc(-c4ccc(C5CCN(C6COC6)CC5)cc4)ccc3[nH]c2=O)c1] | Ex. 2 |
| 100 | 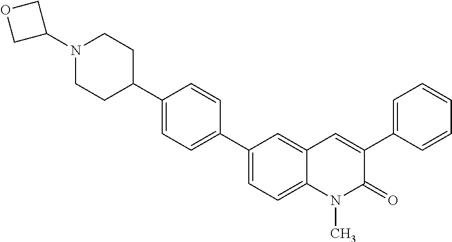 [SMILES: Cn1c(=O)c(-c2ccccc2)cc2cc(-c3ccc(C4CCN(C5COC5)CC4)cc3)ccc21] | Ex. 5 |
| 101 | 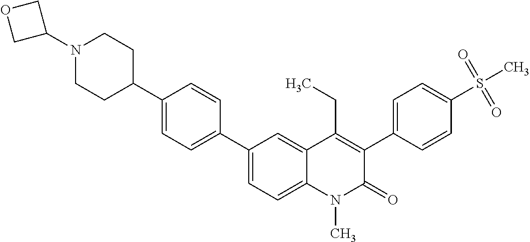 [SMILES: CCc1c(-c2ccc(S(C)(=O)=O)cc2)c(=O)n(C)c2ccc(-c3ccc(C4CCN(C5COC5)CC4)cc3)cc12] | Ex. 5 |

| Ex. No. | Preparative HPLC method; Analytical LC/MS method; Yield; ¹H NMR |
|---|---|
| 4 | Prep Method 1: 23.9 mg, 41% yield; Analytical Method 5: Purity: 98.6%; |
| | Observed Mass: 502.0; Retention Time: 1.25 min; Analytical Method 6: |
| | Purity: 96.9%; Observed Mass: 502.0; Retention Time: 1.52 min; ¹H NMR |
| | (500 MHz, DMSO-d₆) δ 12.09 (s, 1H), 8.30 (s, 1H), 8.07-8.02 (m, 2H), |
| | 8.01-7.96 (m, 3H), 7.83 (dd, J = 8.7, 1.6 Hz, 1H), 7.57 (d, J = 8.6 Hz, 2H), |
| | 7.40 (d, J = 8.6 Hz, 1H), 7.02 (d, J = 8.7 Hz, 2H), 3.25 (s, 3H), 3.22-3.14 |
| | (m, 4H), 2.74-2.67 (m, 1H), 2.65-2.58 (m, 4H), 1.02 (d, J = 6.5 Hz, 6H). |
| 5 | Prep Method 2: 4.9 mg, 15% yield; Analytical Method 5: Purity: 99.1%; |
| | Observed Mass: 481.1; Retention Time: 1.29 min; Analytical Method 6: |
| | Purity: 97.2%; Observed Mass: 481.1; Retention Time: 1.60 min; ¹H NMR |
| | (500 MHz, DMSO-d₆) δ 11.95 (s, 1H), 10.04 (s, 1H), 8.12 (s, 1H), 7.97 (d, |
| | J = 1.8 Hz, 1H), 7.78 (dd, J = 8.5, 2.0 Hz, 1H), 7.74 (d, J = 8.7 Hz, 2H), |
| | 7.68-7.61 (m, 4H), 7.38 (d, J = 8.6 Hz, 1H), 7.12 (br d, J = 8.6 Hz, 3H), |
| | 3.58-3.46 (m, 4H), 3.19-3.01 (m, 4H), 2.07 (s, 3H), 1.34-1.28 (m, 6H). |
| 6 | Prep Method 2: 9.4 mg, 32% yield; Analytical Method 5: Purity: 97.4%; |
| | Observed Mass: 425.2; Retention Time: 0.96 min; Analytical Method 6: |
| | Purity: 93.2%; Observed Mass: 425.2; Retention Time: 1.36 min; ¹H NMR |
| | (500 MHz, DMSO-d₆) δ 12.10 (s, 1H), 8.86 (s, 1H), 8.74-8.65 (m, 1H), 8.54 |
| | (d, J = 8.0 Hz, 1H), 8.13 (d, J = 1.5 Hz, 1H), 7.94-7.82 (m, 2H), 7.66 (br d, |
| | J = 8.2 Hz, 2H), 7.45-7.34 (m, 2H), 7.13 (br d, J = 6.9 Hz, 2H), 4.03-3.90 |
| | (m, 2H), 3.67-3.48 (m, 3H), 3.24-3.14 (m, 2H), 3.08-2.97 (m, 2H), 1.37- |
| | 1.23 (m, 6H). |
| 7 | Prep Method 2: 12.7 mg, 32% yield; Analytical Method 5: Purity: 95.7%; |
| | Observed Mass: 492.2; Retention Time: 1.21 min; Analytical Method 6: |
| | Purity: 94.6%; Observed Mass: 492.2; Retention Time: 1.84 min; ¹H NMR |
| | (500 MHz, DMSO-d₆) δ 12.08 (s, 1H), 10.14 (s, 1H), 8.31 (s, 1H), 8.07 (d, |
| | J = 8.6 Hz, 2H), 8.02 (s, 1H), 8.02-7.97 (m, 2H), 7.84 (dd, J = 8.2, 1.4 Hz, |
| | 1H), 7.64 (br d, J = 8.3 Hz, 2H), 7.42 (d, J = 8.5 Hz, 1H), 7.12 (br d, J = 8.7 |
| | Hz, 2H), 3.61-3.46 (m, 4H), 3.29-3.15 (m, 5H), 1.28 (br d, J = 6.4 Hz, 6H); |
| | protons in the range of 2.0 to 4.5 ppm obscured by water suppression. |
| 8 | Prep Method 2: 9.8 mg, 12% yield; Analytical Method 5: Purity: 97.8%; |
| | Observed Mass: 517.1; Retention Time: 1.19 min; Analytical Method 6: |
| | Purity: 100%; Observed Mass: 517.2; Retention Time: 1.79 min; ¹H NMR |
| | (500 MHz, DMSO-d₆) δ 12.00 (s, 1H), 9.80 (s, 1H), 8.13 (s, 1H), 8.01 (d, |
| | J = 1.8 Hz, 1H), 7.81 (dd, J = 8.6, 2.1 Hz, 1H), 7.68-7.60 (m, 3H), 7.50-7.46 |
| | (m, 1H), 7.43-7.38 (m, 2H), 7.26-7.22 (m, 1H), 7.13 (d, J = 8.9 Hz, 2H), |
| | 3.96 (br d, J = 12.9 Hz, 2H), 3.66-3.48 (m, 3H), 3.25-3.11 (m, 3H), 3.04 (s, |
| | 3H), 3.02-2.98 (m, 1H), 1.31 (d, J = 6.7 Hz, 6H). |
| 9 | Prep Method 1: 2.9 mg, 4% yield; Analytical Method 5: Purity: 100%; |
| | Observed Mass: 467.1; Retention Time: 1.2 min; Analytical Method 6: |
| | Purity: 100%; Observed Mass: 467.6; Retention Time: 1.98 min; ¹H NMR |
| | (500 MHz, DMSO-d₆) δ 11.81 (s, 1H), 8.03 (s, 1H), 7.92 (d, J = 1.9 Hz, |
| | 1H), 7.73-7.68 (m, 3H), 7.56 (d, J = 8.9 Hz, 2H), 7.34 (d, J = 8.6 Hz, 1H), |
| | 7.02 (d, J = 8.9 Hz, 2H), 6.77 (d, J = 9.0 Hz, 2H), 3.20-3.15 (m, 4H), 2.95 |
| | (s, 6H), 2.72-2.64 (m, 1H), 2.61-2.56 (m, 4H), 1.01 (d, J = 6.5 Hz, 6H). |
| 10 | Prep Method 2: 16.5 mg, 51% yield; Analytical Method 5: Purity: 100%; |
| | Observed Mass: 478.1; Retention Time: 1.59 min; Analytical Method 6: |
| | Purity: 100%; Observed Mass: 478.1; Retention Time: 1.59 min; ¹H NMR |
| | (500 MHz, DMSO-d₆) δ 12.11 (s, 1H), 8.13 (s, 1H), 7.98 (d, J = 1.4 Hz, |
| | 1H), 7.85 (dd, J = 8.6, 1.8 Hz, 1H), 7.76-7.67 (m, 1H), 7.66-7.57 (m, 3H), |
| | 7.41 (d, J = 8.5 Hz, 1H), 7.12 (d, J = 8.9 Hz, 2H), 3.59-3.50 (m, 1H), 1.30 |
| | (d, J = 6.6 Hz, 6H); protons in the range of 2.0 to 4.5 ppm obscured by |
| | water suppression. |
| 11 | Prep Method 1: 8.3 mg, 10% yield; Analytical Method 5: Purity: 93.3%; |
| | Observed Mass: 503.2; Retention Time: 1.11 min; Analytical Method 6: |
| | Purity: 100%; Observed Mass: 503.3; Retention Time: 1.28 min; ¹H NMR |
| | (500 MHz, DMSO-d₆) δ 8.26 (s, 1H), 7.98 (s, 1H), 7.97-7.92 (m, 2H), 7.87 |
| | (d, J = 8.3 Hz, 2H), 7.83-7.78 (m, 1H), 7.57 (br d, J = 8.6 Hz, 2H), 7.40 (br |
| | d, J = 8.7 Hz, 1H), 7.01 (br d, J = 8.7 Hz, 2H), 3.55-3.47 (m, 1H), 3.19-3.12 |
| | (m, 4H), 2.69-2.62 (m, 1H), 2.57 (br d, J = 4.0 Hz, 4H), 1.00 (d, J = 6.5 Hz, |
| | 6H); NH₂ protons broad. |
| 12 | Prep Method 2: 34.9 mg, 50% yield; Analytical Method 5: Purity: 98.3%; |
| | Observed Mass: 449.4; Retention Time: 1.45 min; Analytical Method 6: |
| | Purity: 100%; Observed Mass: 449.3; Retention Time: 1.82 min; ¹H NMR |
| | (500 MHz, DMSO-d₆) δ 12.11 (s, 1H), 8.31 (s, 1H), 8.24 (s, 1H), 8.13 (br d, |
| | J = 7.9 Hz, 1H), 8.00 (s, 1H), 7.84 (br dd, J = 6.3, 2.4 Hz, 2H), 7.73-7.66 |
| | (m, 1H), 7.66-7.61 (m, 2H), 7.41 (d, J = 8.5 Hz, 1H), 7.12 (br d, J = 8.7 Hz, |
| | 2H), 4.03-3.52 (m, 6H), 3.24-2.99 (m, 3H), 1.30 (br d, J = 6.6 Hz, 6H). |
| 13 | Prep Method 2: 21.3 mg, 47% yield; Analytical Method 5: Purity: 100%; |
| | Observed Mass: 537.2; Retention Time: 1.13 min; Analytical Method 6: |
| | Purity: 100%; Observed Mass: 537.1; Retention Time: 1.56 min; ¹H NMR |
| | (500 MHz, DMSO-d₆) δ 12.07 (s, 1H), 8.95-8.72 (m, 1H), 8.28 (s, 1H), 8.13 |
| | (d, J = 7.9 Hz, 2H), 8.03 (s, 1H), 7.99-7.92 (m, 2H), 7.84 (br d, J = 8.3 Hz, |
| | 1H), 7.64 (d, J = 8.5 Hz, 2H), 7.42 (d, J = 8.5 Hz, 1H), 7.12 (br d, J = 8.4 |
| | Hz, 2H), 4.59-4.48 (m, 2H), 3.92-3.52 (m, 8H), 3.25-3.13 (m, 2H), 2.73- |
| | 2.66 (m, 4H), 1.30 (d, J = 6.5 Hz, 6H). |
| 15 | Prep Method 1: 16.4 mg, 52% yield; Analytical Method 5: Purity: 95.7%; |
| | Observed Mass: 449.0; Retention Time: 1.32 min; Analytical Method 6: |
| | Purity: 95.2%; Observed Mass: 449.0; Retention Time: 1.59 min; ¹H NMR |
| | (500 MHz, DMSO-d₆) δ 12.13 (s, 1H), 8.15 (s, 1H), 7.98 (d, J = 1.8 Hz, |
| | 1H), 7.96-7.90 (m, 1H), 7.86 (dd, J = 8.7, 2.0 Hz, 1H), 7.83-7.76 (m, 1H), |
| | 7.66 (d, J = 7.8 Hz, 1H), 7.64-7.59 (m, 1H), 7.57 (d, J = 8.9 Hz, 2H), 7.41 |
| | (d, J = 8.6 Hz, 1H), 7.03 (d, J = 8.9 Hz, 2H), 3.21-3.14 (m, 4H), 2.71-2.64 |
| | (m, 1H), 2.63-2.55 (m, 4H), 1.01 (d, J = 6.5 Hz, 6H). |
| 16 | Prep Method 2: 9.9 mg, 12% yield; Analytical Method 5: Purity: 100%; |
| | Observed Mass: 517.1; Retention Time: 1.29 min; Analytical Method 6: |
| | Purity: 95.1%; Observed Mass: 517.1; Retention Time: 1.64 min; ¹H NMR |
| | (500 MHz, DMSO-d₆) δ 11.95 (s, 1H), 8.13 (s, 1H), 7.97 (s, 1H), 7.80-7.73 |
| | (m, 2H), 7.62 (br d, J = 8.4 Hz, 2H), 7.38 (d, J = 8.5 Hz, 1H), 7.26 (d, J = |
| | 8.5 Hz, 2H), 7.09 (br d, J = 9.0 Hz, 2H), 3.88 (s, 1H), 3.66-3.40 (m, 5H), |
| | 3.16 (br s, 4H), 3.02 (s, 3H), 1.23 (br d, J = 5.7 Hz, 6H) one exchangeable |
| | proton signal broadened. |
| 17 | Prep Method 2: 19.8 mg, 67% yield; Analytical Method 5: Purity: 100%; |
| | Observed Mass: 425.2; Retention Time: 0.94 min; Analytical Method 6: |
| | Purity: 95.2%; Observed Mass: 425.3; Retention Time: 1.27 min; ¹H NMR |
| | (500 MHz, DMSO-d₆) δ 8.96-8.69 (m, 2H), 8.54 (s, 1H), 8.18 (br s, 2H), |
| | 8.05 (s, 1H), 7.95-7.87 (m, 1H), 7.64 (br d, J = 8.5 Hz, 2H), 7.44 (br d, J = |
| | 8.5 Hz, 1H), 7.12 (br d, J = 8.4 Hz, 2H), 4.17-2.50 (m, 8H), 1.75-1.44 (m, |
| | 1H), 1.30 (br d, J = 5.4 Hz, 6H), 1.10-0.87 (m, 1H); protons in the range of |
| | 2.0 to 4.5 ppm obscured by water suppression. |
| 18 | Prep Method 2: 2.8 mg, 12% yield; Analytical Method 5: Purity: 100%; |
| | Observed Mass: 424.2; Retention Time: 1.41 min; Analytical Method 6: |
| | Purity: 100%; Observed Mass: 424.1; Retention Time: 1.81 min; ¹H NMR |
| | (500 MHz, DMSO-d₆) δ 11.97 (s, 1H), 8.15 (s, 1H), 7.99 (s, 1H), 7.80 (dd, |
| | J = 8.8, 1.5 Hz, 1H), 7.76 (d, J = 7.4 Hz, 2H), 7.63 (d, J = 8.5 Hz, 2H), |
| | 7.51-7.42 (m, 2H), 7.39 (d, J = 8.1 Hz, 2H), 7.11 (br d, J = 8.5 Hz, 2H), |
| | 3.49-2.50 (m, 9H), 1.27 (br d, J = 6.0 Hz, 6H); protons in the range of 2.0 to |
| | 4.5 ppm obscured by water suppression. |
| 19 | Prep Method 2: 11.7 mg, 31% yield; Analytical Method 5: Purity: 100%; |
| | Observed Mass: 549.4; Retention Time: 1.59 min; Analytical Method 6: |
| | Purity: 95.7%; Observed Mass: 549.6; Retention Time: 1.92 min; ¹H NMR |
| | (500 MHz, DMSO-d₆) δ 12.02 (s, 1H), 9.66-9.39 (m, 1H), 8.21 (s, 1H), 8.01 |
| | (s, 1H), 7.89-7.76 (m, 3H), 7.64 (d, J = 8.6 Hz, 2H), 7.47 (br d, J = 8.0 Hz, |
| | 2H), 7.40 (d, J = 8.6 Hz, 1H), 7.12 (br d, J = 8.8 Hz, 2H), 3.99-3.87 (m, |
| | 2H), 3.59-3.49 (m, 4H), 3.26-2.93 (m, 4H), 1.86-1.72 (m, 4H), 1.51 (br s, |
| | 6H), 1.31 (d, J = 6.6 Hz, 6H). |
| 20 | Prep Method 1: 4.8 mg, 15% yield; Analytical Method 5: Purity: 94.9%; |
| | Observed Mass: 460.0; Retention Time: 1.43 min; Analytical Method 6: |
| | Purity: 95.2%; Observed Mass: 460.0; Retention Time: 1.77 min; ¹H NMR |
| | (500 MHz, DMSO-d₆) δ 8.12 (s, 1H), 7.95 (d, J = 1.7 Hz, 1H), 7.82 (dd, J = |
| | 8.6, 1.8 Hz, 1H), 7.56 (d, J = 8.6 Hz, 2H), 7.45-7.25 (m, 4H), 7.01 (d, J = |
| | 8.7 Hz, 2H), 3.27-3.01 (m, 4H), 2.70-2.63 (m, 1H), 2.60-2.55 (m, 4H), 1.00 |
| | (d, J = 6.5 Hz, 7H). |
| 21 | Prep Method 2: 7.1 mg, 23% yield; Analytical Method 5: Purity: 98.1%; |
| | Observed Mass: 509.3; Retention Time: 1.33 min; Analytical Method 6: |
| | Purity: 96%; Observed Mass: 509.4; Retention Time: 1.61 min; ¹H NMR |
| | (500 MHz, DMSO-d₆) δ 11.93 (s, 1H), 9.99 (s, 1H), 8.12 (s, 1H), 7.98 (s, |
| | 1H), 7.78 (dd, J = 8.6, 1.8 Hz, 1H), 7.74 (br d, J = 8.6 Hz, 2H), 7.64 (br t, |
| | J = 9.1 Hz, 4H), 7.38 (d, J = 8.5 Hz, 1H), 7.11 (br d, J = 8.7 Hz, 2H), 4.10- |
| | 3.57 (m, 9H), 2.31 (t, J = 7.3 Hz, 2H), 1.70-1.55 (m, 2H), 1.29 (br d, J = 6.6 |
| | Hz, 6H), 0.92 (t, J = 7.4 Hz, 3H). |
| 22 | Prep Method 2: 5.5 mg, 16% yield; Analytical Method 5: Purity: 100%; |
| | Observed Mass: 511.2; Retention Time: 1.56 min; Analytical Method 6: |
| | Purity: 100%; Observed Mass: 511.1; Retention Time: 2.01 min; ¹H NMR |
| | (500 MHz, DMSO-d₆) δ 11.84 (s, 1H), 8.04 (s, 1H), 7.95 (s, 1H), 7.77-7.69 |
| | (m, 3H), 7.63 (br d, J = 8.6 Hz, 2H), 7.36 (br d, J = 8.5 Hz, 1H), 7.11 (br d, |
| | J = 8.5 Hz, 2H), 6.63 (br d, J = 8.5 Hz, 2H), 5.55-5.39 (m, 1H), 3.68-3.44 |
| | (m, 10H), 3.30-3.11 (m, 3H), 2.32-2.10 (m, 2H), 1.30 (br d, J = 6.4 Hz, 6H). |
| 23 | Prep Method 2: 11.7 mg, 30% yield; Analytical Method 5: Purity: 100%; |
| | Observed Mass: 545.2; Retention Time: 1.52 min; Analytical Method 6: |
| | Purity: 97.1%; Observed Mass: 545.2; Retention Time: 1.96 min; ¹H NMR |
| | (500 MHz, DMSO-d₆) δ 8.18 (s, 1H), 8.06 (d, J = 1.8 Hz, 1H), 7.92 (dd, J = |
| | 9.0, 1.9 Hz, 1H), 7.78 (d, J = 8.5 Hz, 2H), 7.68 (d, J = 8.8 Hz, 2H), 7.61 (d, |
| | J = 8.6 Hz, 1H), 7.48 (d, J = 8.5 Hz, 2H), 7.13 (br d, J = 8.8 Hz, 2H), 4.02- |
| | 3.82 (m, 1H), 3.73 (s, 3H), 3.63-3.49 (m, 8H), 3.28 (s, 3H), 2.98 (s, 3H), |
| | 1.30 (d, J = 6.6 Hz, 6H). |
| 24 | Prep Method 2: 13.7 mg, 44% yield; Analytical Method 5: Purity: 99%; |
| | Observed Mass: 460.1; Retention Time: 1.54 min; Analytical Method 6: |
| | Purity: 100%; Observed Mass: 460.2; Retention Time: 1.96 min; ¹H NMR |
| | (500 MHz, DMSO-d₆) δ 12.10 (s, 1H), 8.14 (s, 1H), 7.99 (s, 1H), 7.85 (br d, |
| | J = 8.7 Hz, 1H), 7.64 (d, J = 8.6 Hz, 2H), 7.48 (q, J = 8.8 Hz, 1H), 7.42 (d, |
| | J = 8.5 Hz, 1H), 7.37-7.32 (m, 1H), 7.31-7.26 (m, 1H), 7.12 (br d, J = 8.7 |
| | Hz, 2H), 1.30 (d, J = 6.6 Hz, 6H); protons in the range of 2.0 to 4.5 ppm |
| | obscured by water suppression. |
| 25 | Prep Method 2: 2.1 mg, 7% yield; Analytical Method 5: Purity: 100%; |
| | Observed Mass: 438.0; Retention Time: 1.49 min; Analytical Method 6: |
| | Purity: 100%; Observed Mass: 438.0; Retention Time: 1.80 min; ¹H NMR |
| | (500 MHz, DMSO-d₆) δ 7.94 (s, 1H), 7.88 (s, 1H), 7.82-7.76 (m, 1H), 7.68- |
| | 7.58 (m, 2H), 7.41 (d, J = 9.1 Hz, 1H), 7.31-7.07 (m, 6H), 3.84-3.72 (m, |
| | 10H), 2.17 (s, 3H), 1.28-1.21 (m, 6H); protons in the range of 2.0 to 4.5 |
| | ppm obscured by water suppression. |
| 26 | Prep Method 2: 2.0 mg, 4% yield; Analytical Method 5: Purity: 95.8%; |
| | Observed Mass: 522.9; Retention Time: 1.63 min; Analytical Method 6: |
| | Purity: 95.5%; Observed Mass: 523.4; Retention Time: 1.99 min; ¹H NMR |
| | (500 MHz, DMSO-d₆) δ 8.11 (s, 1H), 8.05-7.99 (m, 1H), 7.91-7.84 (m, 1H), |
| | 7.68 (br d, J = 9.8 Hz, 4H), 7.61 (br d, J = 8.5 Hz, 3H), 7.03 (br d, J = 8.8 Hz, |
| | 2H), 3.72 (s, 3H), 3.58 (br s, 2H), 3.23-3.11 (m, 4H), 2.65 (s, 2H), 1.12 (d, |
| | J = 6.9 Hz, 6H), 1.01 (d, J = 6.6 Hz, 6H). |
| 27 | Prep Method 2: 2.4 mg, 2% yield; Analytical Method 5: Purity: 100%; |
| | Observed Mass: 438.0; Retention Time: 1.49 min; Analytical Method 6: |
| | Purity: 100%; Observed Mass: 438.0; Retention Time: 1.80 min; ¹H NMR |
| | (500 MHz, DMSO-d₆) δ 8.29 (s, 1H), 8.10 (d, J = 1.9 Hz, 1H), 8.03-7.99 |
| | (m, 4H), 7.98-7.94 (m, 1H), 7.69 (d, J = 8.5 Hz, 2H), 7.63 (d, J = 8.9 Hz, |
| | 1H), 7.14 (d, J = 8.6 Hz, 2H), 4.07-3.87 (m, 2H), 3.74 (s, 3H), 3.64-3.49 |
| | (m, 7H), 3.26 (s, 3H), 1.31 (d, J = 6.6 Hz, 6H). |
| 28 | Prep Method 2: 4.1 mg, 1% yield; Analytical Method 5: Purity: 100%; |
| | Observed Mass: 482.2; Retention Time: 1.71 min; Analytical Method 6: |
| | Purity: 100%; Observed Mass: 482.1; Retention Time: 1.88 min; ¹H NMR |
| | (500 MHz, DMSO-d₆) δ 12.06 (s, 1H), 8.27 (s, 1H), 8.06-7.99 (m, 3H), 7.93 |
| | (d, J = 8.4 Hz, 2H), 7.82 (dd, J = 8.5, 1.5 Hz, 1H), 7.63 (d, J = 8.8 Hz, 2H), |
| | 7.41 (d, J = 8.5 Hz, 1H), 7.10 (br d, J = 8.9 Hz, 2H), 3.87 (s, 3H), 3.79-3.67 |
| | (m, 3H), 3.35-2.81 (m, 6H), 1.26 (br d, J = 6.6 Hz, 6H). |
| 29 | Prep Method 1: 7 mg, 9% yield; Analytical Method 5: Purity: 100%; |
| | Observed Mass: 517.0; Retention Time: 1.38 min; Analytical Method 6: |
| | Purity: 100%; Observed Mass: 517.1; Retention Time: 1.74 min; ¹H NMR |
| | (500 MHz, DMSO-d₆) δ 12.28 (s, 1H), 8.95 (s, 1H), 8.13 (s, 1H), 8.01 (s, |
| | 1H), 7.87 (br d, J = 8.3 Hz, 1H), 7.65 (d, J = 8.4 Hz, 2H), 7.50-7.39 (m, |
| | 4H), 7.36-7.30 (m, 1H), 7.11 (br d, J = 8.5 Hz, 2H), 3.98-3.58 (m, 8H), |
| | 3.56-3.48 (m, 1H), 2.87 (s, 3H), 1.29 (d, J = 6.6 Hz, 6H); protons in the |
| | range of 2.0 to 4.5 ppm obscured by water suppression. |
| 30 | Prep Method 1: 3.4 mg, 11% yield; Analytical Method 5: Purity: 94.4%; |
| | Observed Mass: 454.2; Retention Time: 1.40 min; Analytical Method 6: |
| | Purity: 93.1%; Observed Mass: 454.1; Retention Time: 1.86 min; ¹H NMR |
| | (500 MHz, DMSO-d₆) δ 11.94 (s, 1H), 8.17 (s, 1H), 7.96 (s, 1H), 7.78 (dd, |
| | J = 8.5, 1.7 Hz, 1H), 7.56 (d, J = 8.8 Hz, 2H), 7.39-7.32 (m, 4H), 7.01 (br d, |
| | J = 8.8 Hz, 2H), 6.98-6.94 (m, 1H), 3.81 (s, 3H), 3.24-3.11 (m, 4H), 2.71- |
| | 2.64 (m, 1H), 2.61-2.55 (m, 4H), 1.01 (d, J = 6.5 Hz, 6H). |
| 31 | Prep Method 1: 2.4 mg, 8% yield; Analytical Method 5: Purity: 95.7%; |
| | Observed Mass: 442.1; Retention Time: 1.57 min; Analytical Method 6: |
| | Purity: 91.4%; Observed Mass: 442.1; Retention Time: 1.57 min; ¹H NMR |
| | (500 MHz, DMSO-d₆) δ 12.26-11.71 (m, 1H), 8.15 (s, 1H), 7.95 (s, 1H), |
| | 7.84-7.74 (m, 3H), 7.57 (d, J = 8.6 Hz, 2H), 7.38 (d, J = 8.7 Hz, 1H), 7.25 |
| | (t, J = 8.8 Hz, 2H), 7.02 (d, J = 8.7 Hz, 2H), 3.30-3.13 (m, 4H), 2.81-2.73 |
| | (m, 1H), 2.71-2.59 (m, 4H), 1.04 (d, J = 6.5 Hz, 6H). |
| 32 | Prep Method 1: 7.4 mg, 26% yield; Analytical Method 5: Purity: 100%; |
| | Observed Mass: 425.0; Retention Time: 0.99 min; Analytical Method 6: |
| | Purity: 100%; Observed Mass: 425.0; Retention Time: 0.99 min; ¹H NMR |
| | (500 MHz, DMSO-d₆) δ 9.89 (s, 1H), 8.92 (d, J = 1.5 Hz, 1H), 8.56 (dd, J = |
| | 4.7, 1.5 Hz, 1H), 8.27 (s, 1H), 8.21-8.14 (m, 1H), 7.98 (d, J = 1.6 Hz, 1H), |
| | 7.85-7.77 (m, 1H), 7.57 (br d, J = 8.6 Hz, 2H), 7.47 (dd, J = 8.1, 5.0 Hz, |
| | 1H), 7.40 (d, J = 8.6 Hz, 1H), 7.02 (br d, J = 8.9 Hz, 2H), 3.22-3.05 (m, |
| | 4H), 2.70-2.63 (m, 1H), 2.63-2.55 (m, 4H), 1.01 (d, J = 6.5 Hz, 6H). |
| 33 | Prep Method 1: 7.4 mg, 22% yield; Analytical Method 5: Purity: 99.4%; |
| | Observed Mass: 492.1; Retention Time: 1.47 min; Analytical Method 6: |
| | Purity: 100%; Observed Mass: 492.0; Retention Time: 1.84 min; ¹H NMR |
| | (500 MHz, DMSO-d₆) δ 11.97 (br s, 1H), 7.93 (d, J = 1.5 Hz, 1H), 7.89 (s, |
| | 1H), 7.85-7.78 (m, 2H), 7.76-7.71 (m, 1H), 7.66-7.60 (m, 1H), 7.56 (d, J = |
| | 8.8 Hz, 2H), 7.45 (d, J = 7.4 Hz, 1H), 7.39 (d, J = 8.5 Hz, 1H), 7.01 (d, J = |
| | 8.7 Hz, 2H), 3.23-3.08 (m, 4H), 2.73-2.62 (m, 1H), 2.60-2.56 (m, 4H), 1.00 |
| | (d, J = 6.5 Hz, 6H). |
| 34 | Prep Method 1: 5.4 mg, 19% yield; Analytical Method 5: Purity: 100%; |
| | Observed Mass: 426.1; Retention Time: 1.19 min; Analytical Method 6: |
| | Purity: 96.2%; Observed Mass: 426.1; Retention Time: 1.52 min; ¹H NMR |
| | (500 MHz, DMSO-d₆) δ 12.28-12.11 (m, 1H), 9.22-9.11 (m, 3H), 8.41 (s, |
| | 1H), 7.98 (s, 1H), 7.89-7.82 (m, 1H), 7.58 (d, J = 8.9 Hz, 2H), 7.42 (d, J = |
| | 8.7 Hz, 1H), 7.04 (br d, J = 8.6 Hz, 2H), 3.49-3.12 (m, 5H), 2.96-2.59 (m, |
| | 4H), 1.04 (br d, J = 6.4 Hz, 6H). |
| 35 | Prep Method 1: 2.7 mg, 8% yield; Analytical Method 5: Purity: 93.3%; |
| | Observed Mass: 478.4; Retention Time: 1.37 min; Analytical Method 6: |
| | Purity: 91.5%; Observed Mass: 478.3; Retention Time: 1.57 min; ¹H NMR |
| | (500 MHz, DMSO-d₆) δ 12.02 (s, 1H), 8.33 (s, 1H), 8.03 (s, 1H), 7.91 (d, |
| | J = 7.9 Hz, 1H), 7.83-7.78 (m, 1H), 7.69-7.59 (m, 3H), 7.48-7.39 (m, 2H), |
| | 7.15 (t, J = 7.6 Hz, 1H), 7.10 (br d, J = 8.6 Hz, 2H), 4.09 (s, 3H), 3.82-3.63 |
| | (m, 4H), 3.57-3.43 (m, 1H), 1.29 (d, J = 6.6 Hz, 6H). |
| 36 | Prep Method 2: 4.5 mg, 6% yield; Analytical Method 5: Purity: 100%; |
| | Observed Mass: 490.0; Retention Time: 1.56 min; Analytical Method 6: |
| | Purity: 98.9%; Observed Mass: 490.0; Retention Time: 1.91 min; ¹H NMR |
| | (500 MHz, DMSO-d₆) δ 11.99 (s, 1H), 8.17 (s, 1H), 7.96 (s, 1H), 7.85 (d, |
| | J = 8.7 Hz, 2H), 7.79-7.72 (m, 1H), 7.57 (d, J = 8.7 Hz, 2H), 7.38 (d, J = 8.5 |
| | Hz, 1H), 7.25 (d, J = 8.7 Hz, 2H), 7.29 (t, J = 74.1 Hz, 1H), 7.02 (br d, J = |
| | 8.7 Hz, 2H), 3.24-3.14 (m, 4H), 2.78-2.70 (m, 1H), 2.70-2.58 (m, 4H), 1.03 |
| | (br d, J = 6.3 Hz, 6H). |
| 37 | Prep Method 1: 1.8 mg, 5% yield; Analytical Method 5; Purity: 91.6%; |
| | Observed Mass: 492.0; Retention Time: 1.46 min; Analytical Method 6: |
| | Purity: 97.1%; Observed Mass: 492.0; Retention Time: 1.84 min; ¹H NMR |
| | (500 MHz, DMSO-d₆) δ 12.06 (s, 1H), 8.00-7.93 (m, 2H), 7.84 (dd, J = 8.7, |
| | 1.9 Hz, 1H), 7.63-7.52 (m, 3H), 7.50-7.37 (m, 2H), 7.02 (br d, J = 8.9 Hz, |
| | 2H), 2.72-2.64 (m, 1H), 2.63-2.55 (m, 4H), 1.01 (d, J = 6.5 Hz, 6H); |
| | protons in the range of 2.0 to 4.5 ppm obscured by water suppression. |
| 38 | Prep Method 1: 14.6 mg, 44% yield; Analytical Method 5: Purity: 99.4%; |
| | Observed Mass: 492.0; Retention Time: 1.59 min; Analytical Method 6: |
| | Purity: 100%; Observed Mass: 492.0; Retention Time: 2.01 min; ¹H NMR |
| | (500 MHz, DMSO-d₆) δ 12.02 (s, 1H), 7.99 (s, 1H), 7.94 (s, 1H), 7.82 (dd, |
| | J = 8.7, 1.6 Hz, 1H), 7.71 (d, J = 1.8 Hz, 1H), 7.56 (d, J = 8.7 Hz, 2H), |
| | 7.52-7.45 (m, 2H), 7.39 (d, J = 8.7 Hz, 1H), 7.01 (d, J = 8.7 Hz, 2H), 3.21- |
| | 3.12 (m, 4H), 2.70-2.64 (m, 1H), 2.60-2.55 (m, 4H), 1.00 (d, J = 6.5 Hz, 6H). |
| 39 | Prep Method 1: 4.5 mg, 15% yield; Analytical Method 5: Purity: 99.1%; |
| | Observed Mass: 458.1; Retention Time: 1.57 min; Analytical Method 6: |
| | Purity: 99.1%; Observed Mass: 458.0; Retention Time: 1.94 min; ¹H NMR |
| | (500 MHz, DMSO-d₆) δ 11.99 (s, 1H), 8.19 (s, 1H), 7.96 (s, 1H), 7.85-7.76 |
| | (m, 3H), 7.56 (d, J = 8.7 Hz, 2H), 7.50 (d, J = 8.5 Hz, 2H), 7.38 (d, J = 8.5 |
| | Hz, 1H), 7.01 (d, J = 8.8 Hz, 2H), 3.22-3.12 (m, 4H), 2.69-2.62 (m, 1H), |
| | 2.60-2.55 (m, 4H), 1.00 (d, J = 6.6 Hz, 6H). |
| 40 | Prep Method 2: 23.1 mg, 56% yield; Analytical Method 5: Purity: 98.2%; |
| | Observed Mass: 514.1; Retention Time: 1.62 min; Analytical Method 6: |
| | Purity: 96%; Observed Mass: 514.1; Retention Time: 1.97 min; ¹H NMR |
| | (500 MHz, DMSO-d₆) δ 12.30 (s, 1H), 8.27 (s, 1H), 8.01 (s, 1H), 7.94-7.87 |
| | (m, 1H), 7.64 (br d, J = 8.5 Hz, 2H), 7.45 (d, J = 8.4 Hz, 1H), 7.12 (br d, J = |
| | 8.1 Hz, 2H), 4.07-3.48 (m, 5H), 3.26-2.85 (m, 4H), 1.30 (br d, J = 5.7 Hz, 6H). |
| 41 | Prep Method 1: 3.2 mg, 10% yield; Analytical Method 5: Purity: 100%; |
| | Observed Mass: 478.0; Retention Time: 1.42 min; Analytical Method 6: |
| | Purity: 98.8%; Observed Mass: 478.0; Retention Time: 1.77 min; ¹H NMR |
| | (500 MHz, DMSO-d₆) δ 12.12 (s, 1H), 8.13 (s, 1H), 7.95 (s, 1H), 7.85 (dd, |
| | J = 8.7, 1.6 Hz, 1H), 7.56 (d, J = 8.5 Hz, 2H), 7.41 (d, J = 8.7 Hz, 1H), 7.30 |
| | (br t, J = 8.5 Hz, 2H), 7.01 (d, J = 8.7 Hz, 2H), 3.20-3.13 (m, 4H), 2.72- |
| | 2.63 (m, 1H), 2.61-2.55 (m, 4H), 1.01 (d, J = 6.5 Hz, 6H); protons in the |
| | range of 2.0 to 4.5 ppm obscured by water suppression. |
| 42 | Prep Method 1: 3.2 mg, 10% yield; Analytical Method 5: Purity: 100%; |
| | Observed Mass: 442.1; Retention Time: 1.50 min; Analytical Method 6: |
| | Purity: 97%; Observed Mass: 442.0; Retention Time: 1.72 min; ¹H NMR |
| | (500 MHz, DMSO-d₆) δ 12.00 (s, 1H), 8.06 (s, 1H), 7.95 (d, J = 1.9 Hz, |
| | 1H), 7.81 (dd, J = 8.6, 2.1 Hz, 1H), 7.57 (d, J = 8.9 Hz, 2H), 7.53 (td, J = |
| | 7.7, 1.6 Hz, 1H), 7.49-7.42 (m, 1H), 7.39 (d, J = 8.6 Hz, 1H), 7.32-7.25 (m, |
| | 2H), 7.02 (d, J = 9.0 Hz, 2H), 3.20-3.15 (m, 4H), 2.71-2.65 (m, 1H), 2.61- |
| | 2.56 (m, 4H), 1.01 (d, J = 6.5 Hz, 6H). |
| 43 | Prep Method 1: 19.1 mg, 21% yield; Analytical Method 3: Purity: 100%; |
| | Observed Mass: 484.2; Retention Time: 1.31 min; ¹H NMR (500 MHz, |
| | CHLOROFORM-d) δ 10.08 (br s, 1H), 7.93 (s, 1H), 7.78 (d, J = 1.9 Hz, |
| | 1H), 7.70 (dd, J = 8.5, 1.9 Hz, 1H), 7.55 (d, J = 8.7 Hz, 2H), 7.45 (d, J = 1.9 |
| | Hz, 1H), 7.37 (dd, J = 8.3, 2.0 Hz, 1H), 7.28 (s, 1H), 7.03 (d, J = 8.7 Hz, |
| | 2H), 6.99 (d, J = 8.5 Hz, 1H), 3.98 (s, 3H), 3.96 (s, 3H), 3.35-3.25 (m, 4H), |
| | 2.85-2.67 (m, 5H), 1.12 (d, J = 6.5 Hz, 6H). |
| 44 | Prep Method 1: 5.1 mg, 51% yield; Analytical Method 5: Purity: 99.2%; |
| | Observed Mass: 458.0; Retention Time: 1.49 min; Analytical Method 6: |
| | Purity: 100%; Observed Mass: 458.0; Retention Time: 1.92 min; ¹H NMR |
| | (500 MHz, DMSO-d₆) δ 11.94 (br d, J = 1.9 Hz, 1H), 7.97-7.92 (m, 2H), |
| | 7.80 (dd, J = 8.7, 1.6 Hz, 1H), 7.57 (d, J = 8.8 Hz, 2H), 7.54-7.49 (m, 1H), |
| | 7.44-7.38 (m, 4H), 7.01 (d, J = 8.8 Hz, 2H), 3.22-3.13 (m, 4H), 2.70-2.67 |
| | (m, 1H), 2.68-2.56 (m, 4H), 1.01 (d, J = 6.4 Hz, 6H). |
| 45 | Prep Method 1: 6.0 mg, 18% yield; Analytical Method 5: Purity: 98.3%; |
| | Observed Mass: 492.0; Retention Time: 1.63 min; Analytical Method 6: |
| | Purity: 95.3%; Observed Mass: 492.0; Retention Time: 2.01 min; ¹H NMR |
| | (500 MHz, DMSO-d₆) δ 12.05 (s, 1H), 8.31 (s, 1H), 8.17 (s, 1H), 8.08 (br d, |
| | J = 7.9 Hz, 1H), 8.00 (s, 1H), 7.82 (br d, J = 6.6 Hz, 1H), 7.73 (s, 1H), 7.71- |
| | 7.66 (m, 1H), 7.58 (br d, J = 8.9 Hz, 2H), 7.40 (d, J = 8.5 Hz, 1H), 7.03 (br |
| | d, J = 8.5 Hz, 2H), 3.24-3.11 (m, 4H), 2.76-2.57 (m, 4H), 1.02 (br d, J = 6.3 |
| | Hz, 6H); protons in the range of 2.0 to 4.5 ppm obscured by water |
| | suppression. |
| 46 | Prep Method 1: 15.6 mg, 45% yield; Analytical Method 5: Purity: 100%; |
| | Observed Mass: 508.1; Retention Time: 1.66 min; Analytical Method 6: |
| | Purity: 100%; Observed Mass: 508.0; Retention Time: 2.08 min; ¹H NMR |
| | (500 MHz, DMSO-d₆) δ 12.18-11.82 (m, 1H), 8.28 (s, 1H), 7.99 (s, 1H), |
| | 7.86-7.75 (m, 3H), 7.68-7.52 (m, 3H), 7.44-7.34 (m, 2H), 7.02 (d, J = 8.6 |
| | Hz, 2H), 3.24-2.95 (m, 4H), 2.71-2.62 (m, 1H), 2.61-2.55 (m, 4H), 1.01 (d, |
| | J = 6.5 Hz, 6H). |
| 47 | Prep Method 1: 6.3 mg, 21% yield; Analytical Method 5: Purity: 100%; |
| | Observed Mass: 442.1; Retention Time: 1.59 min; Analytical Method 6: |
| | Purity: 98.9%; Observed Mass: 442.2; Retention Time: 2.0 min; ¹H NMR |
| | (500 MHz, DMSO-d₆) δ 12.02 (br s, 1H), 8.25 (s, 1H), 7.97 (s, 1H), 7.82- |
| | 7.77 (m, 1H), 7.69-7.61 (m, 2H), 7.56 (br d, J = 8.6 Hz, 2H), 7.52-7.45 (m, |
| | 1H), 7.38 (d, J = 8.5 Hz, 1H), 7.24-7.18 (m, 1H), 7.01 (br d, J = 8.6 Hz, |
| | 2H), 3.21-3.12 (m, 4H), 2.70-2.62 (m, 1H), 2.60-2.55 (m, 4H), 1.00 (d, J = |
| | 6.5 Hz, 6H). |
| 48 | Prep Method 1 and 2: 1.0 mg, 3% yield; Analytical Method 5: Purity: |
| | 100%; Observed Mass: 477.2; Retention Time: 1.56 min; Analytical |
| | Method 6: Purity: 100%; Observed Mass: 477.2; Retention Time: 1.9 min; |
| | ¹H NMR (500 MHz, DMSO-d₆) δ 11.95 (s, 1H), 8.42 (s, 1H), 8.34 (s, 1H), |
| | 8.18-8.11 (m, 2H), 7.95 (s, 1H), 7.73 (dd, J = 8.6, 1.7 Hz, 1H), 7.68 (br d, |
| | J = 8.7 Hz, 2H), 7.54 (d, J = 8.0 Hz, 1H), 7.38 (d, J = 8.7 Hz, 1H), 7.30-7.24 |
| | (m, 1H), 7.24-7.18 (m, 1H), 7.12 (br d, J = 8.9 Hz, 2H), 3.88 (s, 3H), 1.30 |
| | (br d, J = 5.2 Hz, 6H); protons in the range of 2.0 to 4.5 ppm obscured by |
| | water suppression, |
| 49 | Prep Method 2: 4.6 mg, 16% yield; Analytical Method 5: Purity: 100%; |
| | Observed Mass: 496.2; Retention Time: 1.13 min; Analytical Method 6: |
| | Purity: 100%; Observed Mass: 496.2; Retention Time: 1.35 min; ¹H NMR |
| | (500 MHz, DMSO-d₆) δ 11.90 (s, 1H), 9.61-9.46 (m, 1H), 8.66 (s, 1H), 8.09 |
| | (s, 1H), 7.96 (s, 1H), 7.76 (br d, J = 8.6 Hz, 1H), 7.69 (br d, J = 8.5 Hz, |
| | 2H), 7.63 (br d, J = 8.5 Hz, 2H), 7.46 (br d, J = 8.6 Hz, 2H), 7.37 (br d, J = |
| | 8.4 Hz, 1H), 7.12 (br d, J = 8.5 Hz, 2H), 6.09 (br d, J = 4.0 Hz, 1H), 4.02- |
| | 3.87 (m, 2H), 3.62-3.47 (m, 3H), 3.25-2.99 (m, 3H), 2.66 (d, J = 4.4 Hz, |
| | 3H), 1.31 (br d, J = 6.4 Hz, 6H). |
| 50 | Prep Method 2: 4.6 mg, 9% yield; Analytical Method 5: Purity: 94.1%; |
| | Observed Mass: 522.2; Retention Time: 1.24 min; Analytical Method 6: |
| | Purity: 93.3%; Observed Mass: 522.2; Retention Time: 1.6 min; ¹H NMR |
| | (500 MHz, DMSO-d₆) δ 10.74 (s, 1H), 8.81-8.72 (m, 1H), 8.22-8.15 (m, |
| | 2H), 8.12-8.07 (m, 1H), 8.07-8.03 (m, 1H), 8.01-7.95 (m, 2H), 7.91-7.85 |
| | (m, 1H), 7.81-7.76 (m, 2H), 7.74-7.67 (m, 1H), 7.65-7.54 (m, 3H), 7.08- |
| | 6.94 (m, 2H), 3.74 (s, 3H), 3.19 (br s, 4H), 2.73-2.66 (m, 1H), 2.60 (br d, |
| | J = 4.3 Hz, 4H), 1.02 (d, J = 6.5 Hz, 6H). |
| 51 | Prep Method 1: 4.0 mg, 9% yield; Analytical Method 5: Purity: 100%; |
| | Observed Mass: 558.3; Retention Time: 1.69 min; Analytical Method 6: |
| | Purity: 96.8%; Observed Mass: 558.0; Retention Time: 2.20 min; ¹H NMR |
| | (500 MHz, DMSO-d₆) δ 11.89 (s, 1H), 8.07 (s, 1H), 7.96 (s, 1H), 7.76 (br |
| | dd, J = 8.4, 1.9 Hz, 1H), 7.72 (br d, J = 8.7 Hz, 2H), 7.63 (br d, J = 8.7 Hz, |
| | 2H), 7.37 (d, J = 8.7 Hz, 1H), 7.11 (br d, J = 8.6 Hz, 2H), 6.85 (br d, J = 8.8 |
| | Hz, 2H), 4.72-3.61 (m, 8H), 3.58-3.47 (m, 5H), 3.02-2.85 (m, 7H), 1.30 (d, |
| | J = 6.6 Hz, 6H). |
| 52 | Prep Method 1: 3.4 mg, 9% yield; Analytical Method 5: Purity: 97.2%; |
| | Observed Mass: 495.1; Retention Time: 1.32 min; Analytical Method 6: |
| | Purity: 90%; Observed Mass: 495.1; Retention Time: 1.5 min; ¹H NMR |
| | (500 MHz, DMSO-d₆) δ 11.93 (s, 1H), 10.00 (s, 1H), 9.73-9.34 (m, 1H), |
| | 8.13 (s, 1H), 7.97 (s, 1H), 7.77 (dd, J = 8.5, 1.7 Hz, 1H), 7.73 (d, J = 8.5 |
| | Hz, 2H), 7.68-7.59 (m, 4H), 7.38 (d, J = 8.5 Hz, 1H), 7.11 (br d, J = 8.8 Hz, |
| | 2H), 4.17-3.59 (m, 4H), 3.33-2.95 (m, 4H), 2.34 (q, J = 7.4 Hz, 2H), 1.30 |
| | (d, J = 6.6 Hz, 6H), 1.09 (t, J = 7.5 Hz, 3H). |
| 53 | Prep Method 2: 12.2 mg, 21% yield; Analytical Method 5: Purity: 99.2%; |
| | Observed Mass: 467.2; Retention Time: 1.25 min; Analytical Method 6: |
| | Purity: 92.8%; Observed Mass: 467.3; Retention Time: 1.41 min; ¹H NMR |
| | (500 MHz, DMSO-d₆) δ 12.01 (s, 1H), 8.23 (s, 1H), 8.06 (br s, 1H), 8.00 (s, |
| | 1H), 7.96-7.91 (m, 2H), 7.85 (d, J = 8.3 Hz, 2H), 7.81 (dd, J = 8.6, 1.6 Hz, |
| | 1H), 7.64 (d, J = 8.6 Hz, 2H), 7.41 (d, J = 8.6 Hz, 1H), 7.37 (br s, 1H), 7.10 |
| | (d, J = 8.7 Hz, 2H), 4.13-3.67 (m, 5H), 3.28-2.83 (m, 4H), 1.29 (d, J = 6.6 |
| | Hz, 6H). |
| 54 | Prep Method 2: 45.7 mg, 67% yield; Analytical Method 5: Purity: 96.5%; |
| | Observed Mass: 481.4; Retention Time: 1.27 min; Analytical Method 6: |
| | Purity: 90.8%; Observed Mass: 481.3; Retention Time: 1.46 min; ¹H NMR |
| | (500 MHz, DMSO-d₆) δ 12.03 (s, 1H), 9.58-9.29 (m, 1H), 8.59-8.42 (m, |
| | 1H), 8.24 (s, 1H), 8.01 (s, 1H), 7.92-7.85 (m, 4H), 7.82 (dd, J = 8.5, 1.8 Hz, |
| | 1H), 7.64 (d, J = 8.8 Hz, 2H), 7.41 (d, J = 8.5 Hz, 1H), 7.12 (br d, J = 8.6 |
| | Hz, 2H), 4.00-3.85 (m, 2H), 3.61-3.52 (m, 3H), 3.25-3.09 (m, 2H), 2.99 (s, |
| | 1H), 2.81 (d, J = 4.4 Hz, 3H), 1.30 (d, J = 6.6 Hz, 6H). |
| 55 | Prep Method 2: 13 mg, 37% yield; Analytical Method 5: Purity: 97.2%; |
| | Observed Mass: 537.2; Retention Time: 1.21 min; Analytical Method 6: |
| | Purity: 95.2%; Observed Mass: 537.2; Retention Time: 1.7 min; ¹H NMR |
| | (500 MHz, DMSO-d₆) δ 12.04 (s, 1H), 8.22 (s, 1H), 8.00 (s, 1H), 7.86 (d, |
| | J = 8.2 Hz, 2H), 7.82 (dd, J = 8.5, 1.7 Hz, 1H), 7.64 (d, J = 8.7 Hz, 2H), 7.49 |
| | (d, J = 8.2 Hz, 2H), 7.40 (d, J = 8.6 Hz, 1H), 7.12 (br d, J = 8.8 Hz, 2H), |
| | 4.01-3.50 (m, 12H), 2.99 (s, 5H), 1.30 (d, J = 6.6 Hz, 6H). |
| 56 | Prep Method 2: 12.4 mg, 39% yield; Analytical Method 5: Purity: 96.1%; |
| | Observed Mass: 537.2; Retention Time: 1.26 min; Analytical Method 6: |
| | Purity: 97.8%; Observed Mass: 507.2; Retention Time: 1.54 min; ¹H NMR |
| | (500 MHz, DMSO-d₆) δ 11.95 (s, 1H), 8.15 (s, 1H), 7.98 (s, 1H), 7.83-7.76 |
| | (m, 3H), 7.71 (d, J = 8.7 Hz, 2H), 7.63 (d, J = 8.2 Hz, 2H), 7.39 (d, J = 8.6 |
| | Hz, 1H), 7.11 (br d, J = 8.5 Hz, 2H), 3.87 (t, J = 7.1 Hz, 2H), 3.45-3.22 (m, |
| | 9H), 2.53 (d, J = 9.2 Hz, 2H), 2.08 (quin, J = 7.6 Hz, 2H), 1.29 (d, J = 6.6 |
| | Hz, 6H). |
| 57 | Prep Method 2: 10.3 mg, 20% yield; Analytical Method 5: Purity: 100%; |
| | Observed Mass: 495.1; Retention Time: 1.37 min; Analytical Method 6: |
| | Purity: 95.3%; Observed Mass: 495.1; Retention Time: 1.77 min; ¹H NMR |
| | (500 MHz, DMSO-d₆) δ 10.08 (s, 1H), 8.11 (s, 1H), 8.04 (d, J = 2.0 Hz, |
| | 1H), 7.89 (dd, J = 8.8, 2.0 Hz, 1H), 7.72-7.61 (m, 6H), 7.58 (d, J = 8.9 Hz, |
| | 1H), 7.12 (d, J = 8.8 Hz, 2H), 4.77-3.73 (m, 4H), 3.71 (s, 3H), 3.57-3.55 |
| | (m, 1H), 3.27-2.94 (m, 4H), 2.07 (s, 3H), 1.30 (d, J = 6.6 Hz, 6H). |
| 58 | Prep Method 1: 15.3 mg, 25% yield; Analytical Method 5: Purity: 100%; |
| | Observed Mass: 438.2; Retention Time: 1.66 min; Analytical Method 6: |
| | Purity: 100%; Observed Mass: 438.2; Retention Time: 2.17 min; ¹H NMR |
| | (500 MHz, DMSO-d₆) δ 8.14 (s, 1H), 8.06 (d, J = 1.8 Hz, 1H), 7.90 (dd, J = |
| | 8.7, 1.9 Hz, 1H), 7.73 (d, J = 7.2 Hz, 2H), 7.64 (br d, J = 8.5 Hz, 2H), 7.59 |
| | (d, J = 9.0 Hz, 1H), 7.49-7.43 (m, 2H), 7.42-7.37 (m, 1H), 7.08 (br d, J = |
| | 8.4 Hz, 2H), 3.73 (s, 3H), 3.65-3.52 (m, 1H), 3.30-2.58 (m, 8H), 1.12 (br d, |
| | J = 0.9 Hz, 6H); protons in the range of 2.0 to 4.5 ppm obscured by water |
| | suppression. |
| 59 | Prep Method 1: 2.3 mg, 5% yield; Analytical Method 5: Purity: 100%; |
| | Observed Mass: 539.2; Retention Time: 1.37 min; Analytical Method 6: |
| | Purity: 100%; Observed Mass: 539.1; Retention Time: 1.72 min; ¹H NMR |
| | (500 MHz, DMSO-d₆) δ 9.64 (s, 1H), 8.13 (s, 1H), 8.06 (d, J = 2.0 Hz, 1H), |
| | 7.89 (dd, J = 8.8, 2.0 Hz, 1H), 7.83-7.78 (m, 2H), 7.71 (d, J = 8.6 Hz, 2H), |
| | 7.66 (br d, J = 8.6 Hz, 2H), 7.59 (d, J = 8.9 Hz, 1H), 7.10 (br d, J = 8.4 Hz, |
| | 2H), 5.79 (s, 1H), 3.73 (s, 3H), 2.89 (s, 1H), 2.73 (s, 1H), 2.54 (s, 1H), 1.37 |
| | (s, 6H), 1.29-1.16 (m, 6H); protons in the range of 2.0 to 4.5 ppm obscured |
| | by water suppression. |
| 60 | Prep Method 1: 8.8 mg, 28% yield; Analytical Method 5: Purity: 100%; |
| | Observed Mass: 495.2; Retention Time: 1.37 min; Analytical Method 6: |
| | Purity: 98.6%; Observed Mass: 495.2; Retention Time: 1.56 min; ¹H NMR |
| | (500 MHz, DMSO-d₆) δ 12.01 (s, 1H), 8.22 (s, 1H), 7.98 (s, 1H), 7.84 (br d, |
| | J = 7.9 Hz, 2H), 7.80 (br d, J = 8.5 Hz, 1H), 7.57 (br d, J = 8.3 Hz, 2H), |
| | 7.47 (br d, J = 7.9 Hz, 2H), 7.38 (br d, J = 8.8 Hz, 1H), 7.03 (br d, J = 8.5 |
| | Hz, 2H), 3.20 (br s, 4H), 3.03-2.90 (m, 7H), 2.65 (br s, 4H), 1.03 (br d, J = |
| | 6.1 Hz, 6H). |
| 61 | Prep Method 1: 6.1 mg, 18% yield; Analytical Method 5: Purity: 100%; |
| | Observed Mass: 509.3; Retention Time: 1.22 min; Analytical Method 6: |
| | Purity: 100%; Observed Mass: 509.2; Retention Time: 1.87 min; ¹H NMR |
| | (500 MHz, DMSO-d₆) δ 11.78 (s, 1H), 8.01 (s, 1H), 7.91 (s, 1H), 7.69 (br d, |
| | J = 8.6 Hz, 3H), 7.56 (br d, J = 8.6 Hz, 2H), 7.34 (d, J = 8.5 Hz, 1H), 7.00 |
| | (br d, J = 8.9 Hz, 2H), 6.56 (br d, J = 8.7 Hz, 2H), 5.04 (br d, J = 3.4 Hz, |
| | 1H), 4.42 (br s, 1H), 3.43-3.28 (m, 3H), 3.22-3.09 (m, 5H), 2.70-2.55 (m, |
| | 5H), 2.11-1.85 (m, 2H), 1.00 (d, J = 6.5 Hz, 6H). |
| 62 | Prep Method 2: 5.4 mg, 1% yield; Analytical Method 5: Purity: 100%; |
| | Observed Mass: 468.2; Retention Time: 1.31 min; Analytical Method 6: |
| | Purity: 100%; Observed Mass: 468.0; Retention Time: 1.18 min; ¹H NMR |
| | (500 MHz, DMSO-d₆) δ 12.05 (s, 1H), 8.26 (s, 1H), 8.00 (d, J = 8.3 Hz, |
| | 3H), 7.91 (d, J = 8.2 Hz, 2H), 7.82 (br d, J = 9.0 Hz, 1H), 7.63 (d, J = 8.5 |
| | Hz, 2H), 7.41 (d, J = 8.5 Hz, 1H), 7.11 (br d, J = 8.5 Hz, 2H), 3.35-3.20 (m, |
| | 9H), 2.98 (s, 1H), 1.29 (d, J = 6.6 Hz, 6H). |
| 63 | Prep Method 1: 11.2 mg, 37% yield; Analytical Method 5: Purity: 100%; |
| | Observed Mass: 518.6; Retention Time: 1.00 min; Analytical Method 6: |
| | Purity: 100%; Observed Mass: 518.5; Retention Time: 1.23 min; ¹H NMR |
| | (500 MHz, DMSO-d₆) δ 11.63 (s, 1H), 7.57 (s, 1H), 7.39 (d, J = 8.5 Hz, |
| | 1H), 7.27 (d, J = 8.3 Hz, 1H), 6.99 (d, J = 8.2 Hz, 1H), 6.74 (s, 1H), 6.70 (d, |
| | J = 8.1 Hz, 1H), 3.79 (s, 2H), 3.72 (s, 2H), 3.01-2.93 (m, 2H), 2.84 (br d, |
| | J = 10.2 Hz, 2H), 2.72-2.61 (m, 3H), 2.29-2.05 (m, 5H), 1.81-1.61 (m, 6H), |
| | 1.47-1.36 (m, 2H), 1.09 (t, J = 7.5 Hz, 3H), 0.95 (d, J = 6.5 Hz, 6H). |
| 64 | Prep Method 1: 8.4 mg, 19% yield; Analytical Method 5: Purity: 96%; |
| | Observed Mass: 430.3; Retention Time: 0.95 min; Analytical Method 6: |
| | Purity: 97.9%; Observed Mass: 430.2; Retention Time: 1.42 min; ¹H NMR |
| | (500 MHz, DMSO-d₆) δ 11.85 (br s, 1H), 8.04 (s, 1H), 7.74 (br d, J = 7.5 |
| | Hz, 2H), 7.57 (s, 1H), 7.46-7.39 (m, 3H), 7.39-7.34 (m, 1H), 7.26 (br d, J = |
| | 8.5 Hz, 1H), 2.98-2.93 (m, 2H), 2.84-2.78 (m, 2H), 2.70-2.60 (m, 2H), 2.28- |
| | 2.17 (m, 3H), 2.07 (br t, J = 11.2 Hz, 2H), 1.80-1.68 (m, 4H), 1.68-1.59 (m, |
| | 2H), 1.48-1.36 (m, 2H), 0.95 (br d, J = 6.4 Hz, 6H). |
| 65 | Prep Method 2: 2.7 mg, 9% yield; Analytical Method 5: Purity: 95.6%; |
| | Observed Mass: 502.1; Retention Time: 1.28 min; Analytical Method 6: |
| | Purity: 98.6%; Observed Mass: 502.1; Retention Time: 1.37 min; ¹H NMR |
| | (500 MHz, DMSO-d₆) δ 12.10 (s, 1H), 8.27 (s, 1H), 8.06 (s, 1H), 7.97-7.93 |
| | (m, 2H), 7.90-7.84 (m, 3H), 7.69 (d, J = 8.2 Hz, 2H), 7.44 (d, J = 8.8 Hz, |
| | 1H), 7.42 (s, 2H), 7.36 (br d, J = 8.2 Hz, 2H), 3.89-3.69 (m, 3H), 3.12-2.86 |
| | (m, 3H), 2.10-2.02 (m, 2H), 1.98-1.87 (m, 2H), 1.28 (d, J = 6.6 Hz, 6H). |
| 66 | Prep Method 1: 3.2 mg, 24% yield; Analytical Method 5: Purity: 100%; |
| | Observed Mass: 536.6; Retention Time: 0.94 min; Analytical Method 6: |
| | Purity: 100%; Observed Mass: 536.2; Retention Time: 1.12 min; ¹H NMR |
| | (500 MHz, DMSO-d₆) δ 11.83 (br s, 1H), 7.98 (d, J = 8.2 Hz, 2H), 7.62 (s, |
| | 1H), 7.50 (d, J = 8.1 Hz, 2H), 7.45 (br d, J = 8.2 Hz, 1H), 7.30 (d, J = 8.5 |
| | Hz, 1H), 3.27 (s, 3H), 3.00-2.93 (m, 2H), 2.81 (br d, J = 11.0 Hz, 2H), 2.67- |
| | 2.61 (m, 3H), 2.29-2.14 (m, 3H), 2.05 (br t, J = 11.4 Hz, 2H), 1.80-1.62 (m, |
| | 5H), 1.40 (td, J = 11.7, 8.9 Hz, 2H), 1.28-1.24 (m, 2H), 1.09 (br t, J = 7.4 |
| | Hz, 3H), 0.94 (d, J = 6.5 Hz, 6H). |
| 67 | Prep Method 2: 1.9 mg, 6% yield; Analytical Method 5: Purity: 100%; |
| | Observed Mass: 516.1; Retention Time: 1.37 min; Analytical Method 6: |
| | Purity: 100%; Observed Mass: 516.0; Retention Time: 1.33 min; ¹H NMR |
| | (500 MHz, DMSO-d₆) δ 12.03 (s, 1H), 9.86-9.74 (m, 1H), 9.35-9.20 (m, |
| | 1H), 8.14 (s, 1H), 8.05 (s, 1H), 7.84 (br d, J = 8.4 Hz, 1H), 7.70 (d, J = 8.2 |
| | Hz, 2H), 7.62 (s, 1H), 7.51-7.45 (m, 1H), 7.44-7.39 (m, 2H), 7.35 (br d, J = |
| | 8.4 Hz, 2H), 7.23 (br d, J = 7.7 Hz, 1H), 3.19-3.07 (m, 3H), 3.03 (s, 3H), |
| | 2.98-2.86 (m, 2H), 2.11-2.02 (m, 2H), 2.01-1.88 (m, 2H), 1.29 (d, J = 6.6 |
| | Hz, 6H). |
| 68 | Prep Method 1: 4.9 mg, 11% yield; Analytical Method 5: Purity: 95.3%; |
| | Observed Mass: 515.1; Retention Time: 1.47 min; Analytical Method 6: |
| | Purity: 98.1%; Observed Mass: 515.2; Retention Time: 1.66 min; ¹H NMR |
| | (500 MHz, DMSO-d₆) δ 8.29 (s, 1H), 8.13 (d, J = 1.8 Hz, 1H), 8.03-7.95 |
| | (m, 5H), 7.68 (d, J = 8.2 Hz, 2H), 7.65 (d, J = 8.9 Hz, 1H), 7.36 (d, J = 8.0 |
| | Hz, 2H), 3.75 (s, 3H), 3.26 (s, 3H), 2.99 (br d, J = 11.7 Hz, 2H), 2.91-2.79 |
| | (m, 1H), 2.62-2.55 (m, 1H), 2.38 (br t, J = 11.0 Hz, 2H), 1.83 (br d, J = 12.7 |
| | Hz, 2H), 1.76-1.65 (m, 2H), 1.05 (d, J = 6.6 Hz, 6H). |
| 69 | Prep Method 1: 10.3 mg, 39% yield; Analytical Method 5: Purity: 95.3%; |
| | Observed Mass: 448.1; Retention Time: 1.42 min; Analytical Method 6: |
| | Purity: 97.8%; Observed Mass: 448.2; Retention Time: 1.60 min; ¹H NMR |
| | (500 MHz, DMSO-d₆) δ 12.31-12.05 (m, 1H), 8.32 (s, 1H), 8.24 (s, 1H), |
| | 8.13 (d, J = 8.0 Hz, 1H), 8.03 (s, 1H), 7.91-7.83 (m, 2H), 7.67 (t, J = 7.9 |
| | Hz, 1H), 7.62 (d, J = 7.9 Hz, 2H), 7.43 (d, J = 8.6 Hz, 1H), 7.34 (d, J = 8.0 |
| | Hz, 2H), 3.58-3.55 (m, 1H), 2.94-2.87 (m, 2H), 2.79-2.68 (m, 1H), 2.25 (br |
| | t, J = 11.1 Hz, 2H), 1.82-1.75 (m, 2H), 1.72-1.57 (m, 2H), 1.00 (d, J = 6.5 |
| | Hz, 6H). |
| 70 | Prep Method 1: 4.9 mg, 97% yield; Analytical Method 5: Purity: 98.4%; |
| | Observed Mass: 488.1; Retention Time: 1.31 min; Analytical Method 6: |
| | Purity: 97.4%; Observed Mass: 488.0; Retention Time: 1.46 min; ¹H NMR |
| | (500 MHz, DMSO-d₆) δ 8.18-8.12 (m, 1H), 8.03-7.97 (m, 1H), 7.83-7.80 |
| | (m, 1H), 7.79-7.74 (m, 2H), 7.68-7.57 (m, 2H), 7.43-7.38 (m, 1H), 7.38- |
| | 7.32 (m, 2H), 7.30-7.24 (m, 2H), 3.50-3.38 (m, 1H), 3.04 (s, 3H), 2.95-2.86 |
| | (m, 2H), 2.24 (s, 3H), 2.11-1.96 (m, 2H), 1.82-1.62 (m, 4H). |
| 71 | Prep Method 1: 4.0 mg, 7% yield; Analytical Method 5: Purity: 100%; |
| | Observed Mass: 501.1; Retention Time: 1.37 min; Analytical Method 6: |
| | Purity: 100%; Observed Mass: 501.0; Retention Time: 1.35 min; ¹H NMR |
| | (500 MHz, DMSO-d₆) δ 12.26-12.07 (m, 1H), 8.32 (s, 1H), 8.09-8.03 (m, |
| | 3H), 8.02-7.98 (m, 2H), 7.87 (br d, J = 8.6 Hz, 1H), 7.67 (br d, J = 8.2 Hz, |
| | 2H), 7.44 (br d, J = 8.9 Hz, 1H), 7.36 (br d, J = 8.0 Hz, 2H), 3.25 (s, 3H), |
| | 3.20-3.14 (m, 3H), 2.76-2.61 (m, 3H), 1.97-1.91 (m, 2H), 1.84-1.72 (m, |
| | 2H), 1.15 (br d, J = 6.3 Hz, 6H). |
| 72 | Prep Method 2: 10.5 mg, 21% yield; Analytical Method 5: Purity: 98.3%; |
| | Observed Mass: 545.1; Retention Time: 1.35 min; Analytical Method 6: |
| | Purity: 100%; Observed Mass: 545.1; Retention Time: 1.41 min; ¹H NMR |
| | (500 MHz, DMSO-d₆) δ 8.30 (s, 1H), 8.13 (s, 1H), 8.05-7.98 (m, 4H), 7.98- |
| | 7.92 (m, 1H), 7.78 (d, J = 8.9 Hz, 1H), 7.73 (d, J = 8.1 Hz, 2H), 7.38 (br d, |
| | J = 7.9 Hz, 2H), 5.02 (t, J = 5.4 Hz, 1H), 4.44 (br t, J = 6.3 Hz, 2H), 3.82- |
| | 3.68 (m, 3H), 3.25 (s, 3H), 3.09 (br t, J = 12.2 Hz, 2H), 2.95-2.86 (m, 2H), |
| | 2.72 (s, 1H), 2.13-2.02 (m, 2H), 2.01-1.89 (m, 2H), 1.28 (d, J = 6.6 Hz, 6H). |
| 73 | Prep Method 1: 13.8 mg, 23% yield; Analytical Method 5: Purity: 96.5%; |
| | Observed Mass: 437.3; Retention Time: 1.71 min; Analytical Method 6: |
| | Purity: 99%; Observed Mass: 437.4; Retention Time: 1.77 min; ¹H NMR |
| | (500 MHz, DMSO-d₆) δ 8.14 (s, 1H), 8.09 (s, 1H), 7.93 (dd, J = 8.9, 2.0 |
| | Hz, 1H), 7.70 (dd, J = 7.6, 4.0 Hz, 4H), 7.62 (d, J = 8.7 Hz, 1H), 7.47-7.42 |
| | (m, 2H), 7.39 (d, J = 7.3 Hz, 1H), 7.36 (d, J = 8.2 Hz, 2H), 3.72 (s, 4H), |
| | 3.68-3.67 (m, 1H), 3.26-3.08 (m, 3H), 2.78-2.68 (m, 2H), 1.98-1.92 (m, |
| | 2H), 1.87-1.74 (m, 1H), 1.16 (d, J = 6.6 Hz, 6H). |
| 74 | Prep Method 2: 7.8 mg, 20% yield; Analytical Method 5: Purity: 100%; |
| | Observed Mass: 544.4; Retention Time: 1.59 min; Analytical Method 6: |
| | Purity: 96.8%; Observed Mass: 544.2; Retention Time: 1.72 min; ¹H NMR |
| | (500 MHz, DMSO-d₆) δ 8.18 (s, 1H), 8.10 (d, J = 1.8 Hz, 1H), 7.95 (dd, J = |
| | 8.8, 2.0 Hz, 1H), 7.77 (d, J = 8.5 Hz, 2H), 7.73 (d, J = 8.2 Hz, 2H), 7.64 (d, |
| | J = 8.9 Hz, 1H), 7.48 (d, J = 8.6 Hz, 2H), 7.37 (br d, J = 7.8 Hz, 2H), 3.73 |
| | (s, 3H), 3.47-3.43 (m, 4H), 3.28 (s, 3H), 3.18-3.05 (m, 2H), 2.98 (s, 3H), |
| | 2.95-2.87 (m, 1H), 2.10-2.02 (m, 2H), 1.98-1.87 (m, 1H), 1.28 (d, J = 6.6 |
| | Hz, 6H). |
| 75 | Prep Method 1, 2: 7.8 mg, 5% yield; Analytical Method 5: Purity: 99.2%; |
| | Observed Mass: 530.1; Retention Time: 1.39 min; Analytical Method 6: |
| | Purity: 98.7%; Observed Mass: 530.1; Retention Time: 1.42 min; ¹H NMR |
| | (500 MHz, DMSO-d₆) δ 9.14-9.06 (m, 1H), 8.14 (s, 1H), 8.02-7.92 (m, 4H), |
| | 7.53-7.47 (m, 2H), 7.38 (dd, J = 9.0, 2.6 Hz, 1H), 7.15-7.11 (m, 2H), 7.10- |
| | 7.04 (m, 2H), 3.68 (s, 3H), 3.45-3.38 (m, 2H), 3.23 (s, 3H), 3.12-3.02 (m, |
| | 2H), 2.84-2.72 (m, 1H), 2.03-1.79 (m, 5H), 1.27 (d, J = 6.6 Hz, 6H). |
| 76 | Prep Method 2: 14.8 mg, 47% yield; Analytical Method 5: Purity: 100%; |
| | Observed Mass: 473.1; Retention Time: 1.38 min; Analytical Method 6: |
| | Purity: 99.2%; Observed Mass: 473.1; Retention Time: 1.47 min; ¹H NMR |
| | (500 MHz, DMSO-d₆) δ 12.20-12.07 (m, 1H), 8.32 (s, 1H), 8.04 (s, 3H), |
| | 8.01 (s, 2H), 7.92-7.82 (m, 1H), 7.73-7.63 (m, 2H), 7.50-7.42 (m, 1H), |
| | 7.41-7.32 (m, 2H), 3.26 (s, 3H), 3.16-3.05 (m, 1H), 2.83 (s, 4H), 2.51 (br s, |
| | 3H), 2.10-2.00 (m, 2H), 1.96-1.78 (m, 2H). |
| 77 | Prep Method 2: 32.7 mg, 49% yield; Analytical Method 5: Purity: 97.7%; |
| | Observed Mass: 544.1; Retention Time: 1.36 min; Analytical Method 6: |
| | Purity: 96.8%; Observed Mass: 544.6; Retention Time: 1.83 min; ¹H NMR |
| | (500 MHz, DMSO-d₆) δ 8.02 (br s, 1H), 8.01 (d, J = 8.1 Hz, 2H), 7.94 (dd, |
| | J = 8.9, 1.1 Hz, 1H), 7.70 (br d, J = 8.5 Hz, 2H), 7.66 (d, J = 8.9 Hz, 1H), |
| | 7.52 (d, J = 8.1 Hz, 2H), 7.13 (d, J = 8.6 Hz, 2H), 4.11-3.83 (m, 2H), 3.68 |
| | (s, 3H), 3.58-3.51 (m, 5H), 3.29 (s, 3H), 3.21-2.96 (m, 2H), 2.75-2.67 (m, |
| | 2H), 1.30 (d, J = 6.6 Hz, 6H), 1.12 (t, J = 7.4 Hz, 3H). |
| 78 | Prep Method 2: 27.5 mg, 88% yield; Analytical Method 5: Purity: 98.9%; |
| | Observed Mass: 487.4; Retention Time: 1.49 min; Analytical Method 6: |
| | Purity: 98.8%; Observed Mass: 487.4; Retention Time: 1.61 min; ¹H NMR |
| | (500 MHz, DMSO-d₆) δ 8.32-8.27 (m, 1H), 8.17-8.12 (m, 1H), 8.00 (s, 5H), |
| | 7.76-7.71 (m, 2H), 7.69-7.63 (m, 1H), 7.41-7.35 (m, 2H), 3.75 (s, 3H), |
| | 3.57-3.46 (m, 3H), 3.15-3.03 (m, 1H), 2.88 (br s, 4H), 2.55 (s, 1H), 2.51 (br |
| | s, 4H), 2.14-2.00 (m, 2H), 1.98-1.81 (m, 2H). |
| 79 | Prep Method 1: 15.3 mg, 39% yield; Analytical Method 5: Purity: 100%; |
| | Observed Mass: 529.1; Retention Time: 1.50 min; Analytical Method 6: |
| | Purity: 99.3%; Observed Mass: 529.3; Retention Time: 1.60 min; ¹H NMR |
| | (500 MHz, DMSO-d₆) δ 8.35-8.28 (m, 1H), 8.17-8.10 (m, 1H), 8.01 (d, |
| | J = 2.7 Hz, 5H), 7.73-7.64 (m, 3H), 7.42-7.30 (m, 2H), 4.46-4.33 (m, 2H), |
| | 3.26 (s, 3H), 3.04-2.93 (m, 2H), 2.87-2.79 (m, 1H), 2.63-2.55 (m, 1H), |
| | 2.43-2.27 (m, 2H), 1.82 (br s, 2H), 1.74-1.62 (m, 2H), 1.30 (s, 3H), 1.04 (d, |
| | J = 6.6 Hz, 6H). |
| 80 | Prep Method 2: 26 mg, 50% yield; Analytical Method 5: Purity: 99.3%; |
| | Observed Mass: 543.2; Retention Time: 1.61 min; Analytical Method 6: |
| | Purity: 99.3%; Observed Mass: 543.3; Retention Time: 1.72 min; ¹H NMR |
| | (500 MHz, DMSO-d₆) δ 8.36-8.23 (m, 1H), 8.18-8.11 (m, 1H), 8.01 (s, 5H), |
| | 7.76-7.66 (m, 3H), 7.42-7.32 (m, 2H), 4.40-4.25 (m, 2H), 3.58-3.38 (m, |
| | 1H), 3.27 (s, 2H), 3.13 (br d, J = 11.7 Hz, 2H), 2.99-2.91 (m, 2H), 2.07 (br s, |
| | 2H), 2.03-1.90 (m, 2H), 1.80-1.64 (m, 2H), 1.30 (d, J = 6.6 Hz, 6H), 1.01 (t, |
| | J = 7.4 Hz, 3H). |
| 81 | Prep Method 2: 3.1 mg, 7% yield; Analytical Method 5: Purity: 95.6%; |
| | Observed Mass: 480.0; Retention Time: 1.25 min; Analytical Method 6: |
| | Purity: 95%; Observed Mass: 480.1; Retention Time: 1.49 min; ¹H NMR |
| | (500 MHz, DMSO-d₆) δ 11.97 (s, 1H), 10.08 (s, 1H), 9.27-9.11 (m, 1H), |
| | 8.13 (s, 1H), 8.02 (d, J = 1.4 Hz, 1H), 7.80 (dd, J = 8.6, 1.9 Hz, 1H), 7.73 |
| | (d, J = 8.6 Hz, 2H), 7.68 (d, J = 8.2 Hz, 2H), 7.63 (br d, J = 8.7 Hz, 2H), |
| | 7.41 (d, J = 8.5 Hz, 1H), 7.35 (br d, J = 8.3 Hz, 2H), 3.41-2.81 (m, 5H), |
| | 2.13-1.81 (m, 7H), 1.28 (d, J = 6.6 Hz, 6H). |
| 82 | Prep Method 2: 24.1 mg, 41% yield; Analytical Method 5: Purity: 96.7%; |
| | Observed Mass: 559.2; Retention Time: 1.35 min; Analytical Method 6: |
| | Purity: 99.3%; Observed Mass: 559.2; Retention Time: 1.41 min; ¹H NMR |
| | (500 MHz, DMSO-d₆) δ 9.24 (br dd, J = 7.0, 5.8 Hz, 1H), 8.31 (s, 1H), 8.15 |
| | (d, J = 1.9 Hz, 1H), 8.07-7.91 (m, 5H), 7.82-7.69 (m, 3H), 7.38 (d, J = 8.2 |
| | Hz, 2H), 4.41 (br t, J = 7.5 Hz, 2H), 3.57 (br t, J = 6.1 Hz, 2H), 3.53-3.47 |
| | (m, 3H), 3.26 (s, 3H), 3.20-3.07 (m, 2H), 2.99-2.87 (m, 1H), 2.14-2.04 (m, |
| | 2H), 2.01-1.82 (m, 4H), 1.29 (d, J = 6.6 Hz, 6H). |
| 83 | Prep Method 2: 10 mg, 15% yield; Analytical Method 5: Purity: 98.5%; |
| | Observed Mass: 516.1; Retention Time: 1.34 min; Analytical Method 6: |
| | Purity: 99.2%; Observed Mass: 516.1; Retention Time: 1.39 min; ¹H NMR |
| | (500 MHz, DMSO-d₆) δ 12.00 (s, 1H), 9.87 (s, 1H), 9.33-9.17 (m, 1H), 8.14 |
| | (s, 1H), 8.02 (s, 1H), 7.81 (dd, J = 8.5, 1.7 Hz, 1H), 7.76 (d, J = 8.6 Hz, |
| | 2H), 7.68 (d, J = 8.2 Hz, 2H), 7.41 (d, J = 8.6 Hz, 1H), 7.35 (d, J = 8.2 Hz, |
| | 2H), 7.27 (d, J = 8.6 Hz, 2H), 7.19-7.01 (m, 1H), 3.35-3.06 (m, 3H), 3.03 |
| | (s, 3H), 2.96-2.87 (m, 1H), 2.10-2.03 (m, 2H), 1.99-1.89 (m, 2H), 1.28 (d, |
| | J = 6.6 Hz, 6H). |
| 84 | Prep Method 2: 46.5 mg, 50% yield; Analytical Method 3: Purity: 100%; |
| | Observed Mass: 530.2; Retention Time: 1.24 min; Analytical Method 4: |
| | Purity: 97.7%; Observed Mass: 530.2; Retention Time: 1.44 min; ¹H NMR |
| | (500 MHz, DMSO-d₆) δ 11.96 (s, 1H), 8.01 (d, J = 8.5 Hz, 2H), 7.93 (d, J = |
| | 1.7 Hz, 1H), 7.81 (dd, J = 8.6, 1.9 Hz, 1H), 7.59 (d, J = 8.9 Hz, 2H), 7.54 |
| | (d, J = 8.5 Hz, 2H), 7.41 (d, J = 8.6 Hz, 1H), 7.03 (d, J = 8.9 Hz, 2H), 3.30 |
| | (s, 3H), 3.20-3.13 (m, 4H), 2.75-2.66 (m, 3H), 2.63-2.54 (m, 4H), 1.14 (t, |
| | J = 7.6 Hz, 3H), 1.01 (d, J = 6.5 Hz, 6H). |
| 85 | Prep Method 2: 20.8 mg, 40% yield; Analytical Method 5: Purity: 100%; |
| | Observed Mass: 557.2; Retention Time: 1.71 min; Analytical Method 6: |
| | Purity: 99.2%; Observed Mass: 557.3; Retention Time: 1.84 min; ¹H NMR |
| | (500 MHz, DMSO-d₆) δ 8.33-8.27 (m, 1H), 8.17-8.11 (m, 1H), 8.00 (s, 4H), |
| | 7.97-7.92 (m, 1H), 7.78-7.72 (m, 2H), 7.71-7.66 (m, 1H), 7.44-7.35 (m, |
| | 2H), 4.32-4.19 (m, 2H), 3.56-3.44 (m, 2H), 3.26 (s, 3H), 3.17-3.06 (m, 2H), |
| | 2.98-2.90 (m, 1H), 2.51 (br d, J = 1.5 Hz, 4H), 2.28-2.17 (m, 1H), 2.13-2.05 |
| | (m, 2H), 2.02-1.88 (m, 2H), 1.30 (d, J = 6.6 Hz, 6H), 0.96 (br d, J = 6.6 Hz, 6H). |
| 86 | Prep Method 2: 2.8 mg, 17% yield; Analytical Method 5: Purity: 93.5%; |
| | Observed Mass: 543.2; Retention Time: 1.52 min; Analytical Method 6: |
| | Purity: 93.8%; Observed Mass: 543.3; Retention Time: 1.45 min; ¹H NMR |
| | (500 MHz, DMSO-d₆) δ 8.09 (s, 1H), 8.01 (d, J = 8.0 Hz, 2H), 7.98 (br d, |
| | J = 9.4 Hz, 1H), 7.76 (d, J = 8.0 Hz, 2H), 7.70 (d, J = 8.9 Hz, 1H), 7.53 (d, |
| | J = 8.0 Hz, 2H), 7.37 (br d, J = 7.9 Hz, 2H), 3.89 (s, 1H), 3.69 (s, 3H), 3.56- |
| | 3.47 (m, 3H), 3.30 (s, 3H), 3.18-3.07 (m, 2H), 2.97-2.88 (m, 1H), 2.76-2.68 |
| | (m, 2H), 2.10-2.03 (m, 2H), 2.01-1.91 (m, 2H), 1.29 (d, J = 6.6 Hz, 5H), |
| | 1.13 (t, J = 7.5 Hz, 3H). |
| 87 | Prep Method 1: 5 mg, 10% yield; Analytical Method 5; Purity: 100%; |
| | Observed Mass: 529.2; Retention Time: 1.42 min; Analytical Method 6: |
| | Purity: 100%; Observed Mass: 529.2; Retention Time: 1.94 min; ¹H NMR |
| | (500 MHz, DMSO-d₆) δ 8.31 (s, 1H), 8.15 (s, 1H), 8.03-7.97 (m, 5H), 7.70 |
| | (br d, J = 8.0 Hz, 2H), 7.67 (d, J = 8.8 Hz, 1H), 7.39 (br d, J = 8.2 Hz, 2H), |
| | 4.68-4.36 (m, 4H), 3.76 (s, 3H), 3.39-3.35 (m, 4H), 3.27 (s, 3H), 2.02-1.63 |
| | (m, 6H); protons in the range of 2.0 to 4.5 ppm obscured by water |
| | suppression. |
| 88 | Prep Method 1: 24.9 mg, 9% yield; Analytical Method 3: Purity: 98.3%; |
| | Observed Mass: 466.1; Retention Time: 1.12 min; ¹H NMR (500 MHz, |
| | DMSO-d₆) δ 11.86 (br s, 1H), 8.04 (s, 1H), 7.98 (d, J = 2.1 Hz, 1H), 7.75 |
| | (dd, J = 8.5, 2.1 Hz, 1H), 7.71 (d, J = 8.9 Hz, 2H), 7.62 (d, J = 8.3 Hz, 2H), |
| | 7.37 (d, J = 8.6 Hz, 1H), 7.34 (d, J = 8.2 Hz, 2H), 6.78 (d, J = 9.0 Hz, 2H), |
| | 2.95 (s, 6H), 2.89 (br d, J = 11.4 Hz, 2H), 2.72 (quin, J = 6.6 Hz, 1H), 2.22 |
| | (td, J = 11.5, 1.9 Hz, 2H), 1.78 (br d, J = 12.3 Hz, 2H), 1.64 (qd, J = 12.2, |
| | 3.5 Hz, 2H), 1.00 (d, J = 6.5 Hz, 6H); protons in the range of 2.0 to 4.5 ppm |
| | obscured by water suppression. |
| 89 | Prep Method 1,2: 39.6 mg, 80% yield; Analytical Method 5: Purity: 100%; |
| | Observed Mass: 573.2; Retention Time: 1.54 min; Analytical Method 6: |
| | Purity: 100%; Observed Mass: 573.1; Retention Time: 1.54 min; ¹H NMR |
| | (500 MHz, DMSO-d₆) δ 8.31 (s, 1H), 8.09 (d, J = 1.8 Hz, 1H), 8.03-7.97 |
| | (m, 5H), 7.94-7.88 (m, 1H), 7.73 (d, J = 8.2 Hz, 2H), 7.37 (br d, J = 7.8 Hz, |
| | 2H), 7.24-7.07 (m, 1H), 4.77 (s, 1H), 4.48-4.37 (m, 1H), 3.41-3.33 (m, 3H), |
| | 3.25 (s, 3H), 3.11 (br t, J = 12.1 Hz, 2H), 2.97-2.88 (m, 1H), 2.12-2.01 (m, |
| | 2H), 2.00-1.85 (m, 2H), 1.28 (d, J = 6.6 Hz, 6H), 1.22 (s, 6H); protons in |
| | the range of 2.0 to 4.5 ppm obscured by water suppression. |
| 90 | Prep Method 2, 1: 13.9 mg, 8% yield; Analytical Method 5: Purity: 100%; |
| | Observed Mass: 517.0; Retention Time: 1.19 min; Analytical Method 6: |
| | Purity: 100%; Observed Mass: 517.1; Retention Time: 1.38 min; ¹H NMR |
| | (500 MHz, DMSO-d₆) δ 11.86 (s, 1H), 8.14 (s, 1H), 8.04 (d, J = 8.5 Hz, |
| | 2H), 7.95 (d, J = 8.4 Hz, 2H), 7.84 (s, 1H), 7.28-7.17 (m, 3H), 7.01 (d, J = |
| | 8.9 Hz, 2H), 6.89 (d, J = 8.9 Hz, 2H), 3.23 (s, 3H), 3.12-3.02 (m, 4H), 2.85- |
| | 2.56 (m, 5H), 1.05 (br d, J = 6.3 Hz, 6H). |
| 91 | Prep Method 1: 2.5 mg, 15% yield; Analytical Method 5: Purity: 95.9%; |
| | Observed Mass: 503.1; Retention Time: 1.55 min; Analytical Method 6: |
| | Purity: 95.2%; Observed Mass: 503.1; Retention Time: 2.07 min; ¹H NMR |
| | (500 MHz, DMSO-d₆) δ 12.02 (s, 1H), 8.18 (s, 1H), 8.02 (s, 1H), 7.83 (br d, |
| | J = 8.6 Hz, 3H), 7.65 (d, J = 8.2 Hz, 2H), 7.44-7.38 (m, 1H), 7.35 (d, J = |
| | 8.0 Hz, 2H), 7.28-7.21 (m, 3H), 4.65-4.58 (m, 2H), 4.54 (br t, J = 5.7 Hz, |
| | 2H), 3.45-3.43 (m, 2H), 3.11-2.88 (m, 2H), 2.69-2.59 (m, 1H), 2.26-2.08 |
| | (m, 1H), 1.95-1.67 (m, 4H). |
| 92 | Prep Method 1: 3.6 mg, 11% yield; Analytical Method 5: Purity: 98.8%; |
| | Observed Mass: 516.1; Retention Time: 1.12 min; Analytical Method 6: |
| | Purity: 99.2%; Observed Mass: 516.1; Retention Time: 1.62 min; ¹H NMR |
| | (500 MHz, DMSO-d₆) δ 12.10 (s, 1H), 8.27 (s, 1H), 8.05 (s, 1H), 7.99-7.94 |
| | (m, 2H), 7.89 (s, 2H), 7.85 (br dd, J = 8.7, 1.6 Hz, 1H), 7.63 (d, J = 8.2 Hz, |
| | 2H), 7.43 (d, J = 8.5 Hz, 1H), 7.41 (s, 2H), 7.35 (d, J = 8.2 Hz, 2H), 4.59- |
| | 4.51 (m, 2H), 4.45 (t, J = 6.2 Hz, 2H), 2.81 (br d, J = 11.2 Hz, 2H), 2.57- |
| | 2.54 (m, 2H), 1.88 (br t, J = 11.0 Hz, 2H), 1.82-1.76 (m, 2H), 1.76-1.56 (m, |
| | 2H); protons in the range of 2.0 to 4.5 ppm obscured by water suppression. |
| 93 | Prep Method 2, 1: 50 mg, 19% yield; Analytical Method 5: Purity: 100%; |
| | Observed Mass: 475.1; Retention Time: 1.40 min; Analytical Method 6: |
| | Purity: 99.1%; Observed Mass: 475.1; Retention Time: 1.49 min; ¹H NMR |
| | (500 MHz, DMSO-d₆) δ 12.18 (s, 1H), 8.98 (dd, J = 12.2, 1.8 Hz, 2H), 8.58 |
| | (d, J = 1.9 Hz, 1H), 8.48 (s, 1H), 8.31 (dd, J = 8.8, 2.0 Hz, 1H), 8.16 (d, J = |
| | 8.7 Hz, 1H), 8.10 (d, J = 1.8 Hz, 1H), 7.88 (dd, J = 8.6, 2.1 Hz, 1H), 6.53 |
| | (s, 1H), 4.33 (t, J = 5.1 Hz, 1H), 3.45-3.42 (m, 1H), 2.90 (br d, J = 11.5 Hz, |
| | 2H), 2.75-2.69 (m, 1H), 2.27-2.19 (m, 2H), 1.79 (br d, J = 12.1 Hz, 2H), |
| | 1.70-1.62 (m, 2H), 1.06 (t, J = 7.0 Hz, 3H), 1.00 (d, J = 6.5 Hz, 6H). |
| 94 | Prep Method 1: 8.2 mg, 26% yield; Analytical Method 5: Purity: 100%; |
| | Observed Mass: 489.3; Retention Time: 1.16 min; Analytical Method 6: |
| | Purity: 100%; Observed Mass: 489.3; Retention Time: 1.59 min; ¹H NMR |
| | (500 MHz, DMSO-d₆) δ 12.17 (s, 1H), 8.97 (d, J = 10.7 Hz, 2H), 8.57 (s, |
| | 1H), 8.47 (s, 1H), 8.29 (d, J = 8.6 Hz, 1H), 8.16 (d, J = 8.9 Hz, 1H), 8.10 (s, |
| | 1H), 7.91-7.84 (m, 1H), 7.65 (d, J = 7.9 Hz, 2H), 7.46 (d, J = 8.5 Hz, 1H), |
| | 7.37 (d, J = 7.9 Hz, 2H), 4.57-4.53 (m, 2H), 4.46 (t, J = 6.1 Hz, 2H), 3.36- |
| | 3.31 (m, 1H), 2.82 (br d, J = 10.2 Hz, 2H), 2.60-2.55 (m, 1H), 1.95-1.84 (m, |
| | 2H), 1.83-1.76 (m, 2H), 1.75-1.65 (m, 2H). |
| 95 | Prep Method 1: 58.3 mg, 51% yield; Analytical Method 3: Purity: 98.5%; |
| | Observed Mass: 512.2; Retention Time: 1.34 min; Analytical Method 4: |
| | Purity: 98.6%; Observed Mass: 512.2; Retention Time: 1.56 min; ¹H NMR |
| | (500 MHz, DMSO-d₆) δ 11.77 (s, 1H), 7.88 (d, J = 1.7 Hz, 1H), 7.76 (dd, |
| | J = 8.6, 1.9 Hz, 1H), 7.58 (d, J = 8.9 Hz, 2H), 7.39 (d, J = 8.6 Hz, 1H), 7.02 |
| | (dd, J = 8.5, 4.1 Hz, 3H), 6.80 (d, J = 1.9 Hz, 1H), 6.75 (dd, J = 8.0, 1.9 Hz, |
| | 1H), 3.81 (s, 3H), 3.74 (s, 3H), 3.21-3.14 (m, 4H), 2.77-2.65 (m, 3H), 2.62- |
| | 2.55 (m, 4H), 1.15 (t, J = 7.6 Hz, 3H), 1.01 (d, J = 6.5 Hz, 6H). |
| 96 | Prep Method 1: 36.5 mg, 39% yield; Analytical Method 3: Purity: 95.5%; |
| | Observed Mass: 480.1; Retention Time: 1.22 min; ¹H NMR (500 MHz, |
| | DMSO-d₆) δ 8.10-7.99 (m, 2H), 7.87 (dd, J = 8.7, 2.1 Hz, 1H), 7.67 (dd, J = |
| | 8.5, 5.4 Hz, 4H), 7.58 (d, J = 8.9 Hz, 1H), 7.36 (d, J = 8.3 Hz, 2H), 6.78 (d, |
| | J = 9.0 Hz, 2H), 3.73 (s, 3H), 3.01-2.89 (m, 8H), 2.85-2.55 (m, 2H), 2.37- |
| | 2.18 (m, 2H), 1.86-1.62 (m, 4H), 1.10-0.96 (m, 6H). |
| 97 | Prep Method 1: 1.0 mg, 10% yield; Analytical Method 5: Purity: 100%; |
| | Observed Mass: 397.2; Retention Time: 1.03 min; Analytical Method 6: |
| | Purity: 90.5%; Observed Mass: 397.1; Retention Time: 1.21 min; ¹H NMR |
| | (500 MHz, DMSO-d₆) δ 8.24-8.21 (m, 1H), 8.05-8.02 (m, 2H), 8.01-7.96 |
| | (m, 2H), 7.66-7.59 (m, 1H), 7.50-7.45 (m, 1H), 7.34-7.28 (m, 1H), 3.42- |
| | 3.38 (m, 2H), 3.25 (s, 3H), 2.98-2.90 (m, 2H), 2.30-2.22 (m, 3H), 2.13-2.09 |
| | (m, 1H), 1.84-1.77 (m, 2H), 1.76-1.67 (m, 2H). |
| 98 | Prep Method 1: 1.6 mg, 5% yield; Analytical Method 5: Purity: 100%; |
| | Observed Mass: 530.3; Retention Time: 1.26 min; Analytical Method 6: |
| | Purity: 100%; Observed Mass: 530.2; Retention Time: 1.63 min; ¹H NMR |
| | (500 MHz, DMSO-d₆) δ 12.10-11.91 (m, 1H), 8.14 (s, 1H), 8.03 (s, 1H), |
| | 7.82 (br d, J = 9.0 Hz, 1H), 7.64 (br d, J = 7.9 Hz, 2H), 7.60 (s, 1H), 7.50- |
| | 7.44 (m, 1H), 7.43-7.38 (m, 2H), 7.35 (br d, J = 7.7 Hz, 2H), 7.22 (br d, J = |
| | 8.1 Hz, 1H), 4.56-4.51 (m, 2H), 4.45 (t, J = 6.0 Hz, 2H), 3.88 (s, 2H), 3.16 |
| | (s, 1H), 3.02 (s, 3H), 2.80 (br d, J = 10.7 Hz, 2H), 1.92-1.84 (m, 2H), 1.82- |
| | 1.76 (m, 2H), 1.74-1.62 (m, 2H). |
| 99 | Prep Method 2: 7.3 mg, 27% yield; Analytical Method 5: Purity: 100%; |
| | Observed Mass: 462.0; Retention Time: 1.35 min; Analytical Method 6: |
| | Purity: 96.6%; Observed Mass: 462.0; Retention Time: 1.86 min; ¹H NMR |
| | (500 MHz, DMSO-d₆) δ 12.15 (s, 1H), 8.32 (s, 1H), 8.24 (s, 1H), 8.13 (d, |
| | J = 8.1 Hz, 1H), 8.05 (s, 1H), 7.90-7.82 (m, 2H), 7.76-7.65 (m, 3H), 7.44 (d, |
| | J = 8.6 Hz, 1H), 7.37 (br d, J = 7.6 Hz, 2H), 4.86-4.66 (m, 4H), 4.46-4.34 |
| | (m, 1H), 3.41-3.32 (m, 2H), 3.03-2.85 (m, 3H), 2.14-2.02 (m, 2H), 2.00- |
| | 1.86 (m, 2H). |
| 100 | Prep Method 1: 28.7 mg, 51% yield; Analytical Method 5: Purity: 100%; |
| | Observed Mass: 451.3; Retention Time: 1.56 min; Analytical Method 6: |
| | Purity: 99.2%; Observed Mass: 451.3; Retention Time: 2.15 min; ¹H NMR |
| | (500 MHz, DMSO-d₆) δ 8.15 (s, 1H), 8.10 (d, J = 1.8 Hz, 1H), 7.94 (dd, J = |
| | 8.8, 1.9 Hz, 1H), 7.72 (d, J = 7.3 Hz, 2H), 7.68 (d, J = 8.1 Hz, 2H), 7.62 (d, |
| | J = 8.9 Hz, 1H), 7.47-7.43 (m, 2H), 7.42-7.31 (m, 3H), 4.60-4.55 (m, 2H), |
| | 4.49 (br t, J = 5.8 Hz, 2H), 3.73 (s, 3H), 3.60-3.50 (m, 1H), 2.95-2.85 (m, |
| | 2H), 2.63-2.55 (m, 1H), 2.09-1.91 (m, 2H), 1.86-1.79 (m, 2H), 1.77-1.66 |
| | (m, 2H). |
| 101 | Prep Method 1: 5.0 mg, 31% yield; Analytical Method 5: Purity: 96.4%; |
| | Observed Mass: 557.1; Retention Time: 1.44 min; Analytical Method 6: |
| | Purity: 95.6%; Observed Mass: 557.2; Retention Time: 1.98 min; ¹H NMR |
| | (500 MHz, DMSO-d₆) δ 8.08 (s, 1H), 8.01 (d, J = 8.2 Hz, 2H), 7.97 (br d, |
| | J = 8.7 Hz, 1H), 7.74-7.67 (m, 3H), 7.53 (d, J = 8.1 Hz, 2H), 7.38 (d, J = 8.2 |
| | Hz, 2H), 4.60-4.55 (m, 2H), 4.52-4.45 (m, 2H), 3.69 (s, 3H), 3.57-3.46 (m, |
| | 3H), 3.30 (s, 3H), 2.94-2.83 (m, 1H), 2.72 (q, J = 7.7 Hz, 2H), 2.65-2.56 |
| | (m, 1H), 2.07-1.92 (m, 1H), 1.85-1.79 (m, 2H), 1.78-1.68 (m, 2H), 1.13 (t, |
| | J = 7.5 Hz, 3H). |

### Analytical LC/MS Methods

Method 1 (TFA, 1 min): Start % B=0, final % B=100 over 1 min gradient; Flow Rate:=1.0 mL/min; Wavelength=254 nm; Solvent A 0.05% TFA in MeCN:H₂O (5:95); Solvent B=0.05% TFA in MeCN:H₂O (95:5); Column=Acquity BEH C18 1.7 μm particles; 2.1×50 mm; Temperature=50° C.

Method 2 (Ammonium Acetate, 1 min): Start % B 0, final % B=100 over 1 min gradient: Flow Rate=1.0 mL/min; Wavelength=220 nm; Solvent A=10 mM NH₄OAc in MeCN:H₂O (5:95); Solvent B=10 mM NH₄OAc in MeCN-H₂O (95:5); Column=Acquity BEH C18 1.7 μm particles; 2.1×50 mm; Temperature=50° C.

Method 3 (TFA, 3 min): Start % B 0, final % B 100 over 3 min gradient; Flow Rate=1.0 mL/min; Wavelength=254 nm; Solvent A=0.05% TFA in MeCN:H₂O (5:95); Solvent B=0.05% TFA in MeCN:H₂O (95:5); Column=Acquity BEH C18 1.7 μm particles; 2.1×50 mm; Temperature=50° C.

Method 4 (Ammonium Acetate, 3 min): Start % B=0, final % B=100 over 3 min gradient; Flow Rate=1.0 mL/min; Wavelength=254 nm; Solvent A=10 mM NH₄OAc in MeCN:H₂O (5:95); Solvent B=10 mM NH₄Ac in MeCN:H₂O (95:5); Column=Acquity BEH C18 1.7 μm particles; 2.1×50 mm; Temperature=50° C.;

Method 5 (SCP TFA, 3 min): Start % B=0, final % B=100 over 3 min gradient; Flow Rate=10 mL/min; Wavelength=254 nm; Solvent A=0.05% TFA in MeCN:H₂O (5:95); Solvent B=0.05% TFA in MeCN:H₂O (95:5); Column=XBridge C18 1.7 μm particles; 2.1×50 mm; Temperature=50° C.

Method 6 (SCP Ammonium Acetate, 3 min): Start % B=0, final % B 100 over 3 min gradient; Flow Rate=1.0 mL/min; Wavelength=254 nm; Solvent A=10 mM NH₄OAc in MeCN:H₂O (5:95); Solvent B=10 mM NH₄OAc in MeCN:H₂O (95:5); Column=XBridge C18 1.7 μm particles; 2.1×50 μm; Temperature=50° C.

### Preparative HPLC Purification Methods:

Method 1 (SCP Ammonium Acetate): Column: XBridge C18, 19 mm×200 mm 5-μm particles; flow rate: 20 mL/min; Mobile Phase A: 10 mM 1 NH₄OAc in MeCN:H₂O (5:95); Mobile Phase B: 10 mM NH₄OAc in MeCN:H₂O (95:5); Column Temperature: 25° C. Fraction collection was triggered by MS signals. Fractions containing the desired product were combined and dried via centrifugal evaporation.

Method 2 (SCP TFA): Column: XBridge C18, 19 mm×200 mm, 5-μm particles; flow rate: 20 mL/min; column Mobile Phase A: 0.05% TFA in MeCN:H₂O (5:95); Mobile Phase B: 0.05% TFA in MeCN:H₂O (95:5); Column Temperature: 25° C. Fraction collection was triggered by MS signals. Fractions containing the desired product were combined and dried via centrifugal evaporation.

Method 3: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 40% B, 40-80% B over 20 minutes, then a 4-minute hold at 100% B: Flow Rate: 20 mL/min; Column Temperature: 25° C. Fraction collection was triggered by MS signals. Fractions containing the desired product were combined and dried via centrifugal evaporation.

Method 4: The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid: Gradient: a 0-minute hold at 32% B, 32-72% B over 20 minutes, then a 4-minute hold at 100% B; Flow Rate: 20 mL/min; Column Temperature: 25° C. Fraction collection was triggered by MS signals. Fractions containing the desired product were combined and dried via centrifugal evaporation.

Method 5: Column: XBridge C18, 200 mm×19 mm, 5-pam particles: Mobile Phase A: 5:95 acetonitrile:water with 10-mM ammonium acetate: Mobile Phase B: 95:5 acetonitrile:water with 10-mM ammonium acetate; Gradient: a 0-minute hold at 34% B, 34-84% B over 20 minutes, then a 4-minute hold at 100% B; Flow Rate: 20 mL/min; Column Temperature: 25° C. Fraction collection was triggered by UV signals. Fractions containing the desired product were combined and dried via centrifugal evaporation.

### Biological Assays

The pharmacological properties of the compounds of this invention may be confirmed by a number of biological assays. The exemplified biological assays, which follow, have been carried out with compounds of the invention.

### TLR7/8/9 Inhibition Reporter Assays

HEK-Blue™-cells (Invivogen) overexpressing human TLR7, TLR8 or TLR9 receptors were used for screening inhibitors of these receptors using an inducible SEAP (secreted embryonic alkaline phosphatase) reporter gene under the control of the IFN-β minimal promoter fused to five NF-κB and AP-1-binding sites. Briefly, cells are seeded into Greiner 384 well plates (15000 cells per well for TLR7, 20,000 for TLR8 and 25,000 for TLR9) and then treated with test compounds in DMSO to yield a final dose response concentration range of 0.05 nM-50 μM. After a 30 minute compound pre-treatment at room temperature, the cells are then stimulated with a TLR7 ligand (gardiquimod at a final concentration of 7.5 μM), TLR8 ligand (R848 at a final concentration of 15.9 μM) or TLR9 ligand (ODN2006 at a final concentration of 5 nM) to activate NF-κB and AP-1 which induce the production of SEAP. After a 22 hour incubation at 37° C., 5% CO₂, SEAP levels are determined with the addition of HEK-Blue™ Detection reagent (Invivogen), a cell culture medium that allows for detection of SEAP, according to manufacturer's specifications. The percent inhibition is determined as the % reduction in the HEK-Blue signal present in wells treated with agonist plus DMSO alone compared to wells treated with a known inhibitor.

| Ex. No. | TLR9 IC₅₀ (nM) | TLR7 IC₅₀ (nM) | TLR8 IC₅₀ (nM) |
|---|---|---|---|
| 4 | 206 | 4,691 | 50,000 |
| 5 | 59 | 3,870 | 50,000 |
| 6 | 79 | 3,832 | 50,000 |
| 7 | 80 | 1,127 | 50,000 |
| 8 | 87 | 299 | 1,819 |
| 9 | 96 | 3,066 | 13,731 |
| 10 | 118 | 50,000 | 50,000 |
| 11 | 119 | 50,000 | 2,050 |
| 12 | 121 | 2,995 | 7,557 |
| 13 | 122 | 4,892 | 50,000 |
| 15 | 259 | 459 | 1,031 |
| 16 | 171 | 17,488 | 50,000 |
| 17 | 171 | 338 | 949 |
| 18 | 190 | 16,667 | 16,347 |
| 19 | 200 | 2,401 | 50,000 |
| 20 | 203 | 1,556 | 5,411 |
| 21 | 214 | 50,000 | 50,000 |
| 22 | 216 | 50,000 | 50,000 |
| 23 | 221 | 5,509 | 35,979 |
| 24 | 224 | 1,998 | 15,242 |
| 25 | 226 | 6,476 | 1,387 |
| 26 | 231 | 1,796 | 673 |
| 27 | 233 | 2,498 | 1,038 |
| 28 | 2,284 | 50,000 | 50,000 |
| 29 | 275 | 5,862 | 1,830 |
| 30 | 277 | 2,162 | 5,215 |
| 31 | 286 | 42,620 | 16,667 |
| 32 | 293 | 418 | 17 |
| 33 | 300 | 2,234 | 4,698 |
| 34 | 307 | 1,082 | — |
| 35 | 314 | 1,292 | 14,134 |
| 36 | 344 | 50,000 | 50,000 |
| 37 | 367 | 5,556 | 50,000 |
| 38 | 376 | 8,879 | 50,000 |
| 39 | 387 | 50,000 | 50,000 |
| 40 | 399 | 50,000 | 50,000 |
| 41 | 411 | 4,772 | 50,000 |
| 42 | 427 | 2,137 | 2,473 |
| 43 | 553 | 5,016 | 50,000 |
| 44 | 701 | 5,611 | 50,000 |
| 45 | 734 | 50,000 | 50,000 |
| 46 | 781 | 50,000 | 50,000 |
| 47 | 857 | 8,615 | 50,000 |
| 48 | 1,161 | 2,153 | 6,879 |
| 49 | 239 | 5,421 | 50,000 |
| 50 | 242 | 15,614 | 50,000 |
| 51 | 242 | 1,343 | 46,951 |
| 52 | 243 | 15,107 | 50,000 |
| 53 | 257 | 4,517 | 1,237 |
| 54 | 379 | 13,023 | 50,000 |
| 55 | 382 | 50,000 | 50,000 |
| 56 | 400 | 5,983 | 50,000 |
| 57 | 615 | 1,220 | 83 |
| 58 | 538 | 32,625 | 1,387 |
| 59 | 652 | — | — |
| 60 | 736 | 50,000 | 50,000 |
| 61 | 1,644 | 50,000 | 50,000 |
| 62 | 1,761 | 50,000 | 50,000 |
| 63 | 27 | 2,815 | 11,013 |
| 64 | 30 | 1,478 | 50,000 |
| 65 | 55 | 342 | 4,866 |
| 66 | 57 | 9,966 | 50,000 |
| 67 | 67 | 817 | 50,000 |
| 68 | 70 | 7,961 | 475 |
| 69 | 75 | 538 | 17,587 |
| 70 | 78 | 204 | 50,000 |
| 71 | 140 | 29,865 | 9,285 |
| 72 | 154 | 3,581 | 21,679 |
| 73 | 219 | 1,476 | 763 |
| 74 | 237 | 11,981 | 29,590 |
| 75 | 253 | 6,630 | 50,000 |
| 76 | 261 | 2,031 | 9,846 |
| 77 | 267 | 10,213 | 361 |
| 78 | 286 | 2,122 | 2,256 |
| 79 | 287 | 5,327 | 25,801 |
| 80 | 298 | 8,381 | 15,139 |
| 81 | 309 | 333 | 796 |
| 82 | 312 | 5,497 | 19,131 |
| 83 | 354 | 369 | 50,000 |
| 84 | 389 | 2,320 | 672 |
| 85 | 400 | 14,618 | 26,303 |
| 86 | 406 | 10,393 | 1,547 |
| 87 | 416 | 50,000 | 29,988 |
| 88 | 449 | — | 118 |
| 89 | 465 | 4,956 | 10,943 |
| 90 | 493 | 3,094 | 50,000 |
| 91 | 512 | 50,000 | 50,000 |
| 92 | 560 | 50,000 | 11,386 |
| 93 | 651 | 1,866 | 49,828 |
| 94 | 689 | 50,000 | 50,000 |
| 95 | 691 | 5,890 | 731 |
| 96 | 1,427 | 17,254 | 50,000 |
| 97 | 960 | 2,512 | 20,142 |
| 98 | 1,433 | 50,000 | 26,718 |
| 99 | 1,654 | 16,667 | 50,000 |
| 100 | 2,271 | 50,000 | 1,904 |
| 101 | 2,370 | 50,000 | 1,339 |

What is claimed is:

1. A compound of Formula (I): 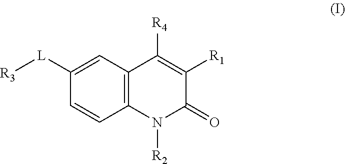
or a salt thereof, wherein:
R₁ is phenyl, pyridinyl, pyrimidinyl, indolyl, indazolyl, or quinoxalinyl, each substituted with zero to 5 R₁ₐ;
each R₁ₐ is independently F, Cl, —CN, —OH, C₁₋₂ alkyl, C₁₋₂ fluoroalkyl, C₁₋₂ alkoxy, C₁₋₂ fluoroalkoxy, —C(O)OH, —C(O)O(C₁₋₂ alkyl), —C(O)NRₓRₓ, —NRₓRₓ, —NRₓC(O)(C₁₋₄ alkyl), —NHC(O)C(CH₃)₂OH, —NHC(O)NRₓRₓ, —NO₂, —S(O)₂(C₁₋₂ alkyl), —S(O)₂NRₓRₓ, —NRₓS(O)₂(C₁₋₂ alkyl), —NRₓC(O)(pyridinyl), —NRₓ(methylazetidinyl), —C(O)(morpholinyl), —C(O)(dimethyl pyrrolidinyl), fluoropyrrolidinyl, hydroxypyrrolidinyl, pyrrolidinonyl, imidazolonyl, or tetrazolyl;
R₂ is hydrogen, C₁₋₄ alkyl, or C₁₋₄ hydroxyalkyl;
L is a bond or —NH—;
R₃ is phenyl or piperidinyl, each substituted with R₃ₐ;
R₃ₐ is —CH₃ or a cyclic group selected from piperidinyl and piperazinyl, wherein said cyclic group is substituted with zero or one R_{3b};
R_{3b} is C₁₋₃ alkyl or oxetanyl;
R₄ is hydrogen or C₁₋₂ alkyl; and
each Rₓ is independently H or —CH₃.

2. The compound according to claim 1 or a salt thereof, wherein:
each R₁ₐ is independently F, Cl, —CN, —OH, —CH₃, —CF₃, —OCH₃, —OCHF₂, —OCF₃, —C(O)OH, —C(O)OCH₃, —C(O)NH₂, —C(O)NH(CH₃), —C(O)N(CH₃)₂, —N(CH₃)₂, —NHC(O)CH₃, —NHC(O)CH₂CH₃, —NHC(O)CH(CH₃)₂, —NHC(O)CH₂CH₂CH₂CH₃, —NHC(O)C(CH₃)₂OH, —NHC(O)NH(CH₃), —NO₂, —S(O)₂CH₃, —S(O)₂NH₂, —NHS(O)₂CH₃, —N(CH₃)S(O)₂CH₃, —NHC(O)(pyridinyl), —C(O)N(CH₃)(oxetanyl), —N(CH₃)(methylazetidinyl), —C(O)(morpholinyl), —C(O)(dimethyl pyrrolidinyl), fluoropyrrolidinyl, hydroxypyrrolidinyl, pyrrolidinonyl, imidazolonyl, or tetrazolyl;
R₂ is hydrogen, —CH₃, —CH₂CH₃, —CH₂CH₂CH₃, —CH₂CH(CH₃)₂, —CH₂CH₂OH, —CH₂CH₂CH₂OH, or —CH₂C(CH₃)₂OH;
L is a bond or —NH—;
R₃ is phenyl or piperidinyl, each substituted with R₃ₐ;
R₃ₐ is —CH₃ or a cyclic group selected from piperidinyl and piperazinyl, wherein said cyclic group is substituted with zero or one R_{3b};
R_{3b} is —CH₃, —CH(CH₃)₃, or oxetanyl; and
R₄ is hydrogen or —CH₂CH₃.

3. The compound according to claim 1 or a salt thereof, wherein R₁ is phenyl, pyridinyl, or pyrimidinyl, each substituted with zero to 5 R₁ₐ;

4. The compound according to claim 1 or a salt thereof, wherein R₁ is phenyl substituted with zero to 5 R₁ₐ.

5. The compound according to claim 1 having the structure of Formula (I) or a salt thereof, wherein L is a bond.

6. The compound according to claim 1 having the structure of Formula (I) or a salt thereof, wherein R₃ is phenyl substituted with zero to 1 R₃ₐ.

7. The compound according to claim 1 having the structure of Formula (I) or a salt thereof, wherein R₃ₐ is a cyclic group selected from piperidinyl and piperazinyl, wherein said cyclic group is substituted with zero or one R_{3b}.

8. The compound according to claim 1 having the structure of Formula (I) or a salt thereof, wherein R₃ is:
(i) piperidinyl or phenyl, each substituted with piperidinyl; or
(ii) phenyl substituted piperazinyl.

9. The compound according to claim 1 having the structure of Formula (I) or a salt thereof, wherein R₃ is:

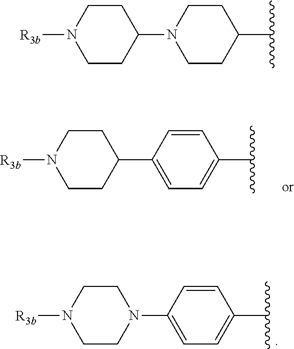

10. The compound according to claim 1 or a salt thereof, wherein R₁ is:

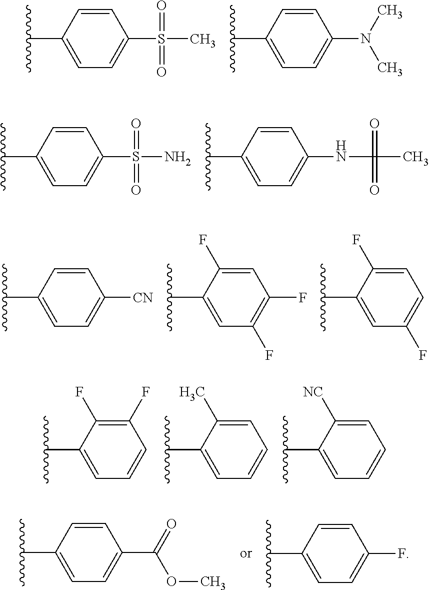

11. The compound according to claim 1 or a salt thereof, wherein said compound is:
6-(4-(4-isopropylpiperazin-1-yl)phenyl)-1-methyl-3-(4-nitrophenyl)quinolin-2(1H)-one (1);
3-(4-hydroxyphenyl)-6-(4-(4-isopropylpiperazin-1-yl)phenyl)quinolin-2(1H)-one (2);
6-((4-(4-isopropylpiperazin-1-yl)phenyl)amino)-1-methyl-3-(4-(methylsulfonyl) phenyl)quinolin-2(1H)-one (3);
3-(4-methanesulfonylphenyl)-6-{4-[4-(propan-2-yl)piperazin-1-yl]phenyl}-1,2-dihydroquinolin-2-one (4);
N-[4-(2-oxo-6-{4-[4-(propan-2-yl)piperazin-1-yl]phenyl}-1,2-dihydroquinolin-3-yl) phenyl]acetamide (5);
6-{4-[4-(propan-2-yl)piperazin-1-yl]phenyl}-3-(pyridin-2-yl)-1,2-dihydroquinolin-2-one (6);
3-(4-(1H-tetrazol-1-yl)phenyl)-6-(4-(4-isopropylpiperazin-1-yl)phenyl) quinolin-2(1H)-one (7);
N-[3-(2-oxo-6-{4-[4-(propan-2-yl)piperazin-1-yl]phenyl}-1,2-dihydroquinolin-3-yl) phenyl]methanesulfonamide (8);
3-[4-(dimethylamino)phenyl]-6-{4-[4-(propan-2-yl)piperazin-1-yl]phenyl}-1,2-dihydroquinolin-2-one (9);
6-{4-[4-(propan-2-yl)piperazin-1-yl]phenyl}-3-(2,4,5-trifluorophenyl)-1,2-dihydroquinolin-2-one (10);
4-(2-oxo-6-{4-[4-(propan-2-yl)piperazin-1-yl]phenyl}-1,2-dihydroquinolin-3-yl) benzene-1-sulfonamide (11);
4-(2-oxo-6-{4-[4-(propan-2-yl)piperazin-1-yl]phenyl}-1,2-dihydroquinolin-3-yl) benzonitrile (12);
N-methyl-N-(oxetan-3-yl)-4-(2-oxo-6-{4-[4-(propan-2-yl)piperazin-1-yl]phenyl}-1,2-dihydroquinolin-3-yl)benzamide (13);
3-[4-(2-oxoimidazolidin-1-yl)phenyl]-6-{4-[4-(propan-2-yl)piperazin-1-yl]phenyl}-1,2-dihydroquinolin-2-one (14);
2-(2-oxo-6-{4-[4-(propan-2-yl)piperazin-1-yl]phenyl}-1,2-dihydroquinolin-3-yl) benzonitrile (15);
N-[4-(2-oxo-6-{4-[4-(propan-2-yl)piperazin-1-yl]phenyl}-1,2-dihydroquinolin-3-yl) phenyl]methanesulfonamide (16);
6-{4-[4-(propan-2-yl)piperazin-1-yl]phenyl}-3-(pyridin-4-yl)-1,2-dihydroquinolin-2-one (17);
3-phenyl-6-{4-[4-(propan-2-yl)piperazin-1-yl]phenyl}-1,2-dihydroquinolin-2-one (18);
3-[4-(2,2-dimethylpyrrolidine-1-carbonyl)phenyl]-6-{4-[4-(propan-2-yl) piperazin-1-yl]phenyl}-1,2-dihydroquinolin-2-one (19);
3-(2,5-difluorophenyl)-6-{4-[4-(propan-2-yl)piperazin-1-yl]phenyl}-1,2-dihydroquinolin-2-one (20);
N-[4-(2-oxo-6-{4-[4-(propan-2-yl)piperazin-1-yl]phenyl}-1,2-dihydroquinolin-3-yl) phenyl]butanamide (21);
3-{4-[(3 S)-3-fluoropyrrolidin-1-yl]phenyl}-6-{4-[4-(propan-2-yl)piperazin-1-yl]phenyl}-1,2-dihydroquinolin-2-one (22);
N-methyl-N-[4-(1-methyl-2-oxo-6-{4-[4-(propan-2-yl)piperazin-1-yl]phenyl}-1,2-dihydroquinolin-3-yl)phenyl]methanesulfonamide (23);
3-(2,3-difluorophenyl)-6-{4-[4-(propan-2-yl)piperazin-1-yl]phenyl}-1,2-dihydroquinolin-2-one (24);
3-(2-methylphenyl)-6-{4-[4-(propan-2-yl)piperazin-1-yl]phenyl}-1,2-dihydroquinolin-2-one (25);
2-methyl-N-[4-(1-methyl-2-oxo-6-{4-[4-(propan-2-yl)piperazin-1-yl]phenyl}-1,2-dihydroquinolin-3-yl)phenyl]propanamide (26);
3-(4-methanesulfonylphenyl)-1-methyl-6-{4-[4-(propan-2-yl)piperazin-1-yl]phenyl}-1,2-dihydroquinolin-2-one (27);
methyl 4-(2-oxo-6-{4-[4-(propan-2-yl)piperazin-1-yl]phenyl}-1,2-dihydroquinolin-3-yl)benzoate (28);
N-[2-(2-oxo-6-{4-[4-(propan-2-yl)piperazin-1-yl]phenyl}-1,2-dihydroquinolin-3-yl) phenyl]methanesulfonamide (29);
3-(3-methoxyphenyl)-6-{4-[4-(propan-2-yl)piperazin-1-yl]phenyl}-1,2-dihydroquinolin-2-one (30);
3-(4-fluorophenyl)-6-{4-[4-(propan-2-yl)piperazin-1-yl]phenyl}-1,2-dihydroquinolin-2-one (31);
6-{4-[4-(propan-2-yl)piperazin-1-yl]phenyl}-3-(pyridin-3-yl)-1,2-dihydroquinolin-2-one (32);
6-{4-[4-(propan-2-yl)piperazin-1-yl]phenyl}-3-[2-(trifluoromethyl)phenyl]-1,2-dihydroquinolin-2-one (33);
6-{4-[4-(propan-2-yl)piperazin-1-yl]phenyl}-3-(pyrimidin-5-yl)-1,2-dihydroquinolin-2-one (34);
3-(1-methyl-1H-indazol-3-yl)-6-{4-[4-(propan-2-yl)piperazin-1-yl]phenyl}-1,2-dihydroquinolin-2-one (35);
3-[4-(difluoromethoxy)phenyl]-6-{4-[4-(propan-2-yl)piperazin-1-yl]phenyl}-1,2-dihydroquinolin-2-one (36);
3-(2,6-dichlorophenyl)-6-{4-[4-(propan-2-yl)piperazin-1-yl]phenyl}-1,2-dihydroquinolin-2-one (37);
3-(2,4-dichlorophenyl)-6-{4-[4-(propan-2-yl)piperazin-1-yl]phenyl}-1,2-dihydroquinolin-2-one (38);
3-(4-chlorophenyl)-6-{4-[4-(propan-2-yl)piperazin-1-yl]phenyl}-1,2-dihydroquinolin-2-one (39);
3-(2,3,4,5,6-pentafluorophenyl)-6-{4-[4-(propan-2-yl)piperazin-1-yl]phenyl}-1,2-dihydroquinolin-2-one (40);
6-{4-[4-(propan-2-yl)piperazin-1-yl]phenyl}-3-(2,4,6-trifluorophenyl)-1,2-dihydroquinolin-2-one (41);
3-(2-fluorophenyl)-6-{4-[4-(propan-2-yl)piperazin-1-yl]phenyl}-1,2-dihydroquinolin-2-one (42);
3-(3,4-dimethoxyphenyl)-6-{4-[4-(propan-2-yl)piperazin-1-yl]phenyl}-1,2-dihydroquinolin-2-one (43);
3-(2-chlorophenyl)-6-{4-[4-(propan-2-yl)piperazin-1-yl]phenyl}-1,2-dihydroquinolin-2-one (44);
6-{4-[4-(propan-2-yl)piperazin-1-yl]phenyl}-3-[3-(trifluoromethyl)phenyl]-1,2-dihydroquinolin-2-one (45);
6-{4-[4-(propan-2-yl)piperazin-1-yl]phenyl}-3-[3-(trifluoromethoxy)phenyl]-1,2-dihydroquinolin-2-one (46);
3-(3-fluorophenyl)-6-{4-[4-(propan-2-yl)piperazin-1-yl]phenyl}-1,2-dihydroquinolin-2-one (47);
3-(1-methyl-1H-indol-3-yl)-6-{4-[4-(propan-2-yl)piperazin-1-yl]phenyl}-1,2-dihydroquinolin-2-one (48);
3-methyl-1-[4-(2-oxo-6-{4-[4-(propan-2-yl)piperazin-1-yl]phenyl}-1,2-dihydroquinolin-3-yl)phenyl]urea (49);
N-[4-(1-methyl-2-oxo-6-{4-[4-(propan-2-yl)piperazin-1-yl]phenyl}-1,2-dihydroquinolin-3-yl)phenyl]pyridine-2-carboxamide (50);
3-{4-[methyl(1-methylazetidin-3-yl)amino]phenyl}-6-{4-[4-(propan-2-yl) piperazin-1-yl]phenyl}-1,2-dihydroquinolin-2-one (51);
N-[4-(2-oxo-6-{4-[4-(propan-2-yl)piperazin-1-yl]phenyl}-1,2-dihydroquinolin-3-yl) phenyl]propanamide (52);
4-(2-oxo-6-{4-[4-(propan-2-yl)piperazin-1-yl]phenyl}-1,2-dihydroquinolin-3-yl) benzamide (53);
N-methyl-4-(2-oxo-6-{4-[4-(propan-2-yl)piperazin-1-yl]phenyl}-1,2-dihydroquinolin-3-yl)benzamide (54);
3-[4-(morpholine-4-carbonyl)phenyl]-6-{4-[4-(propan-2-yl)piperazin-1-yl]phenyl}-1,2-dihydroquinolin-2-one (55);
3-[4-(2-oxopyrrolidin-1-yl)phenyl]-6-{4-[4-(propan-2-yl)piperazin-1-yl]phenyl}-1,2-dihydroquinolin-2-one (56);
N-[4-(1-methyl-2-oxo-6-{4-[4-(propan-2-yl)piperazin-1-yl]phenyl}-1,2-dihydroquinolin-3-yl)phenyl]acetamide (57);
1-methyl-3-phenyl-6-{4-[4-(propan-2-yl)piperazin-1-yl]phenyl}-1,2-dihydroquinolin-2-one (58);
2-hydroxy-2-methyl-N-[4-(1-methyl-2-oxo-6-{4-[4-(propan-2-yl)piperazin-1-yl]phenyl}-1,2-dihydroquinolin-3-yl)phenyl]propanamide (59);
N,N-dimethyl-4-(2-oxo-6-{4-[4-(propan-2-yl)piperazin-1-yl]phenyl}-1,2-dihydroquinolin-3-yl)benzamide (60);
3-{4-[(3R)-3-hydroxypyrrolidin-1-yl]phenyl}-6-{4-[4-(propan-2-yl)piperazin-1-yl]phenyl}-1,2-dihydroquinolin-2-one (61);
4-(2-oxo-6-{4-[4-(propan-2-yl)piperazin-1-yl]phenyl}-1,2-dihydroquinolin-3-yl) benzoic acid (62);
3-(3,4-dimethoxyphenyl)-4-ethyl-6-[1′-(propan-2-yl)-[1,4′-bipiperidine]-4-yl]-1,2-dihydroquinolin-2-one (63);
3-phenyl-6-[1′-(propan-2-yl)-[1,4′-bipiperidine]-4-yl]-1,2-dihydroquinolin-2-one (64);
4-(2-oxo-6-{4-[1-(propan-2-yl)piperidin-4-yl]phenyl}-1,2-dihydroquinolin-3-yl) benzene-1-sulfonamide (65);
4-ethyl-3-(4-methanesulfonylphenyl)-6-[1′-(propan-2-yl)-[1,4′-bipiperidine]-4-yl]-1,2-dihydroquinolin-2-one (66);
N-[3-(2-oxo-6-{4-[1-(propan-2-yl)piperidin-4-yl]phenyl}-1,2-dihydroquinolin-3-yl) phenyl]methanesulfonamide (67);
3-(4-methanesulfonylphenyl)-1-methyl-6-{4-[1-(propan-2-yl)piperidin-4-yl]phenyl}-1,2-dihydroquinolin-2-one (68);
3-(2-oxo-6-{4-[1-(propan-2-yl)piperidin-4-yl]phenyl}-1,2-dihydroquinolin-3-yl) benzonitrile (69);
N-(4-{6-[4-(1-methylpiperidin-4-yl)phenyl]-2-oxo-1,2-dihydroquinolin-3-yl}phenyl) methanesulfonamide (70);
3-(4-methanesulfonylphenyl)-6-{4-[1-(propan-2-yl)piperidin-4-yl]phenyl}-1,2-dihydroquinolin-2-one (71);
1-(2-hydroxyethyl)-3-(4-methanesulfonylphenyl)-6-{4-[1-(propan-2-yl) piperidin-4-yl]phenyl}-1,2-dihydroquinolin-2-one (72);
1-methyl-3-phenyl-6-{4-[1-(propan-2-yl)piperidin-4-yl]phenyl}-1,2-dihydroquinolin-2-one (73);
N-methyl-N-[4-(1-methyl-2-oxo-6-{4-[1-(propan-2-yl)piperidin-4-yl]phenyl}-1,2-dihydroquinolin-3-yl)phenyl]methanesulfonamide (74);
3-(4-methanesulfonylphenyl)-1-methyl-6-({4-[1-(propan-2-yl)piperidin-4-yl]phenyl}amino)-1,2-dihydroquinolin-2-one (75);
3-(4-methanesulfonylphenyl)-6-[4-(1-methylpiperidin-4-yl)phenyl]-1,2-dihydroquinolin-2-one (76);
4-ethyl-3-(4-methanesulfonylphenyl)-1-methyl-6-{4-[4-(propan-2-yl) piperazin-1-yl]phenyl}-1,2-dihydroquinolin-2-one (77);
3-(4-methanesulfonylphenyl)-1-methyl-6-[4-(1-methylpiperidin-4-yl)phenyl]-1,2-dihydroquinolin-2-one (78);
1-ethyl-3-(4-methanesulfonylphenyl)-6-{4-[1-(propan-2-yl)piperidin-4-yl]phenyl}-1,2-dihydroquinolin-2-one (79);
3-(4-methanesulfonylphenyl)-6-{4-[1-(propan-2-yl)piperidin-4-yl]phenyl}-1-propyl-1,2-dihydroquinolin-2-one (80);
N-[4-(2-oxo-6-{4-[1-(propan-2-yl)piperidin-4-yl]phenyl}-1,2-dihydroquinolin-3-yl) phenyl]acetamide (81);
1-(3-hydroxypropyl)-3-(4-methanesulfonylphenyl)-6-{4-[1-(propan-2-yl) piperidin-4-yl]phenyl}-1,2-dihydroquinolin-2-one (82);
N-[4-(2-oxo-6-{4-[1-(propan-2-yl)piperidin-4-yl]phenyl}-1,2-dihydroquinolin-3-yl) phenyl]methanesulfonamide (83);
4-ethyl-3-(4-methanesulfonylphenyl)-6-{4-[4-(propan-2-yl)piperazin-1-yl]phenyl}-1,2-dihydroquinolin-2-one (84);
3-(4-methanesulfonylphenyl)-1-(2-methylpropyl)-6-{4-[1-(propan-2-yl) piperidin-4-yl]phenyl}-1,2-dihydroquinolin-2-one (85);
4-ethyl-3-(4-methanesulfonylphenyl)-1-methyl-6-{4-[1-(propan-2-yl) piperidin-4-yl]phenyl}-1,2-dihydroquinolin-2-one (86);
3-(4-methanesulfonylphenyl)-1-methyl-6-{4-[1-(oxetan-3-yl)piperidin-4-yl]phenyl}-1,2-dihydroquinolin-2-one (87);
3-[4-(dimethylamino)phenyl]-6-{4-[1-(propan-2-yl)piperidin-4-yl]phenyl}-1,2-dihydroquinolin-2-one (88);
1-(2-hydroxy-2-methylpropyl)-3-(4-methanesulfonylphenyl)-6-{4-[1-(propan-2-yl) piperidin-4-yl]phenyl}-1,2-dihydroquinolin-2-one (89);
3-(4-methanesulfonylphenyl)-6-({4-[4-(propan-2-yl)piperazin-1-yl]phenyl}amino)-1,2-dihydroquinolin-2-one (90);
3-[4-(difluoromethoxy)phenyl]-6-{4-[1-(oxetan-3-yl)piperidin-4-yl]phenyl}-1,2-dihydroquinolin-2-one (91);
4-(6-{4-[1-(oxetan-3-yl)piperidin-4-yl]phenyl}-2-oxo-1,2-dihydroquinolin-3-yl) benzene-1-sulfonamide (92);
6-{4-[1-(propan-2-yl)piperidin-4-yl]phenyl}-3-(quinoxalin-6-yl)-1,2-dihydroquinolin-2-one (93);
6-{4-[1-(oxetan-3-yl)piperidin-4-yl]phenyl}-3-(quinoxalin-6-yl)-1,2-dihydroquinolin-2-one (94);
3-(3,4-dimethoxyphenyl)-4-ethyl-6-{4-[4-(propan-2-yl)piperazin-1-yl]phenyl}-1,2-dihydroquinolin-2-one (95);
3-[4-(dimethylamino)phenyl]-1-methyl-6-{4-[1-(propan-2-yl)piperidin-4-yl]phenyl}-1,2-dihydroquinolin-2-one (96);
3-(4-methanesulfonylphenyl)-6-(1-methylpiperidin-4-yl)-1,2-dihydroquinolin-2-one (97);
N-[3-(6-{4-[1-(oxetan-3-yl)piperidin-4-yl]phenyl}-2-oxo-1,2-dihydroquinolin-3-yl) phenyl]methanesulfonamide (98);
3-(6-{4-[1-(oxetan-3-yl)piperidin-4-yl]phenyl}-2-oxo-1,2-dihydroquinolin-3-yl) benzonitrile (99);
1-methyl-6-{4-[1-(oxetan-3-yl)piperidin-4-yl]phenyl}-3-phenyl-1,2-dihydroquinolin-2-one (100); or
4-ethyl-3-(4-methanesulfonylphenyl)-1-methyl-6-{4-[1-(oxetan-3-yl) piperidin-4-yl]phenyl}-1,2-dihydroquinolin-2-one (101).

12. A pharmaceutical composition comprising one or more compounds according to claim 1 and a pharmaceutically acceptable carrier or diluent.

13. A method of treating a disease of disorder, comprising administering to a mammalian patient a compound according to claim 1 or a pharmaceutically acceptable salt thereof, wherein said disease or disorder is pathological fibrosis.

14. A method of treating a disease or disorder, comprising administering to a mammalian patent a compound according to claim 1 or a pharmaceutically acceptable salt thereof, wherein said disease or disorder is inflammatory disease, autoimmune disease, or cancer.

15. (canceled)

16. (canceled)

17. The method according to claim 13, wherein the pathological fibrosis is liver fibrosis, renal fibrosis, biliary fibrosis, or pancreatic fibrosis.

18. A method of treating a disease of disorder, comprising administering to a mammalian patient a compound according to claim 1 or a pharmaceutically acceptable salt thereof, wherein said disease or disorder is nonalcoholic steatohepatitis (NASH), non-alcoholic fatty liver disease (NAFLD), chronic kidney disease, diabetic kidney disease, primary sclerosing cholangitis (PSC), or primary biliary cirrhosis (PBC).

19. A method of treating a disease of disorder, comprising administering to a mammalian patient a compound according to claim 1 or a pharmaceutically acceptable salt thereof, wherein said disease or disorder is idiopathic pulmonary fibrosis (IPF).
